SEQUENCE LISTING (68 sequences)

SEQ ID NO: 1 - protein, 112 aa, Artificial Sequence [sequence omitted]

SEQ ID NO: 2 - protein, 116 aa, Artificial Sequence [sequence omitted]

SEQ ID NO: 3 - protein, 112 aa, Artificial Sequence
  Synthetic Construct
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIK

SEQ ID NO: 4 - protein, 120 aa, Artificial Sequence
  Synthetic Construct
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSS

SEQ ID NO: 5 - protein, 112 aa, Artificial Sequence
  Synthetic Construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIK

SEQ ID NO: 6 - protein, 121 aa, Artificial Sequence
  Synthetic Construct
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSS

SEQ ID NO: 7 - protein, 107 aa, Artificial Sequence
  Synthetic Construct
DIVMTQSPSSLSASVGDRVTITCKASQSVTNDVAWYQQKPGKAPKLLIYYASNRYTGVPSRFSGSGSGTDFTLTISSLQPEDFATYYCQQDYRSPWTFGGGTKVEIK

SEQ ID NO: 8 - protein, 117 aa, Artificial Sequence
  Synthetic Construct
QVQLEESGGGVVRPGRSLRLSCAASGFTFDDYGVNWIRQAPGKGLEWVTIIWGDGRTDYNSALKSRFTVSRNNSNNTLSLQMNSLTTEDTAVYYCVRAPGNRAMEYWGQGVLVTVSS

SEQ ID NO: 9 - protein, 107 aa, Mus musculus
DIQMTQTTSSLSASLGDRVTISCRASQDISNYLNWYQQKPDGTVKLLIYYTSILHSGVPSRFSGSGSGTDYSLTISNLEQEDFATYFCQQGNTLPWTFGGGTKLEIK

SEQ ID NO: 10 - protein, 123 aa, Mus musculus
EVQLVESGGGLVKPGGSLKLSCAASGFAFSIYDMSWVRQTPEKRLEWVAYISSGGGTTYYPDTVKGRFTISRDNAKNTLYLQMSSLKSEDTAMYYCARHSGYGSSYGVLFAYWGQGTLVTVSA

SEQ ID NO: 11 - protein, 107 aa, Homo sapiens [sequence omitted]

SEQ ID NO: 12 - protein, 330 aa, Homo sapiens [sequence omitted]

SEQ ID NO: 13 - protein, 327 aa, Artificial Sequence
  Synthetic Construct
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 14 - protein, 847 aa, Homo sapiens
MHLLGPWLLLLVLEYLAFSDSSKWVFEHPETLYAWEGACVWIPCTYRALDGDLESFILFHNPEYNKNTSKFDGTRLYESTKDGKVPSEQKRVQFLGDKNKNCTLSIHPVHLNDSGQLGLRMESKTEKWMERIHLNVSERPFPPHIQLPPEIQESQEVTLTCLLNFSCYGYPIQLQWLLEGVPMRQAAVTSTSLTIKSVFTRSELKFSPQWSHHGKIVTCQLQDADGKFLSNDTVQLNVKHTPKLEIKVTPSDAIVREGDSVTMTCEVSSSNPEYTTVSWLKDGTSLKKQNTFTLNLREVTKDQSGKYCCQVSNDVGPGRSEEVFLQVQYAPEPSTVQILHSPAVEGSQVEFLCMSLANPLPTNYTWYHNGKEMQGRTEEKVHIPKILPWHAGTYSCVAENILGTGQRGPGAELDVQYPPKKVTTVIQNPMPIREGDTVTLSCNYNSSNPSVTRYEWKPHGAWEEPSLGVLKIQNVGWDNTTIACAACNSWCSWASPVALNVQYAPRDVRVRKIKPLSEIHSGNSVSLQCDFSSSHPKEVQFFWEKNGRLLGKESQLNFDSISPEDAGSYSCWVNNSIGQTASKAWTLEVLYAPRRLRVSMSPGDQVMEGKSATLTCESDANPPVSHYTWFDWNNQSLPYHSQKLRLEPVKVQHSGAYWCQGTNSVGKGRSPLSTLTVYYSPETIGRRVAVGLGSCLAILILAICGLKLQRRWKRTQSQQGLQENSSGQSFFVRNKKVRRAPLSEGPHSLGCYNPMMEDGISYTTLRFPEMNIPRTGDAESSEMQRPPPDCDDTVTYSALHKRQVGDYENVIPDFPEDEGIHYSELIQFGVGERPQAQENVDYVILKH

SEQ ID NO: 15 - protein, 5 aa, Artificial Sequence
  Synthetic Construct
SYWLH

SEQ ID NO: 16 - protein, 17 aa, Artificial Sequence
  Synthetic Construct
YINPRNDYTEYNQNFKD

SEQ ID NO: 17 - protein, 7 aa, Artificial Sequence
  Synthetic Construct
RDITTFY

SEQ ID NO: 18 - protein, 17 aa, Artificial Sequence
  Synthetic Construct
KSSQSVLYSANHKNYLA

SEQ ID NO: 19 - protein, 7 aa, Artificial Sequence
  Synthetic Construct
WASTRES

SEQ ID NO: 20 - protein, 8 aa, Artificial Sequence
  Synthetic Construct
HQYLSSWT

SEQ ID NO: 21 - protein, 5 aa, Artificial Sequence
  Synthetic Construct
RSWMN

SEQ ID NO: 22 - protein, 17 aa, Artificial Sequence
  Synthetic Construct
RIYPGDGDTNYSGKFKG

SEQ ID NO: 23 - protein, 11 aa, Artificial Sequence
  Synthetic Construct
DGSSWDWYFDV

SEQ ID NO: 24 - protein, 16 aa, Artificial Sequence
  Synthetic Construct
RSSQSIVHSVGNTFLE

SEQ ID NO: 25 - protein, 7 aa, Artificial Sequence
  Synthetic Construct
KVSNRFS

SEQ ID NO: 26 - protein, 9 aa, Artificial Sequence
  Synthetic Construct
FQGSQFPYT

SEQ ID NO: 27 - protein, 5 aa, Artificial Sequence
  Synthetic Construct
NYWIH

SEQ ID NO: 28 - protein, 17 aa, Artificial Sequence
  Synthetic Construct
GINPGNNYATYRRKFQG

SEQ ID NO: 29 - protein, 12 aa, Artificial Sequence
  Synthetic Construct
EGYGNYGAWFAY

SEQ ID NO: 30 - protein, 16 aa, Artificial Sequence
  Synthetic Construct
RSSQSLANSYGNTFLS

SEQ ID NO: 31 - protein, 7 aa, Artificial Sequence
  Synthetic Construct
GISNRFS

SEQ ID NO: 32 - protein, 9 aa, Artificial Sequence
  Synthetic Construct
LQGTHQPYT

SEQ ID NO: 33 - protein, 5 aa, Artificial Sequence
  Synthetic Construct
DYGVN

SEQ ID NO: 34 - protein, 16 aa, Artificial Sequence
  Synthetic Construct
IIWGDGRTDYNSALKS

SEQ ID NO: 35 - protein, 9 aa, Artificial Sequence
  Synthetic Construct
APGNRAMEY

SEQ ID NO: 36 - protein, 11 aa, Artificial Sequence
  Synthetic Construct
KASQSVTNDVA

SEQ ID NO: 37 - protein, 7 aa, Artificial Sequence
  Synthetic Construct
YASNRYT

SEQ ID NO: 38 - protein, 9 aa, Artificial Sequence
  Synthetic Construct
QQDYRSPWT

SEQ ID NO: 39 - protein, 5 aa, Mus musculus
IYDMS

SEQ ID NO: 40 - protein, 17 aa, Mus musculus
YISSGGGTTYYPDTVKG

SEQ ID NO: 41 - protein, 14 aa, Mus musculus
HSGYGSSYGVLFAY

SEQ ID NO: 42 - protein, 11 aa, Mus musculus
RASQDISNYLN

SEQ ID NO: 43 - protein, 7 aa, Mus musculus
YTSILHS

SEQ ID NO: 44 - protein, 9 aa, Mus musculus
QQGNTLPWT

SEQ ID NO: 45 - protein, 219 aa, Artificial Sequence
  Synthetic Construct
DIQLTQSPSSLSASVGDRVTMSCKSSQSVLYSANHKNYLAWYQQKPGKAPKLLIYWASTRESGVPSRFSGSGSGTDFTFTISSLQPEDIATYYCHQYLSSWTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 446 aa, Artificial Sequence
  Synthetic Construct
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 443 aa, Artificial Sequence
  Synthetic Construct
QVQLVQSGAEVKKPGSSVKVSCKASGYTFTSYWLHWVRQAPGQGLEWIGYINPRNDYTEYNQNFKDKATITADESTNTAYMELSSLRSEDTAFYFCARRDITTFYWGQGTTVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 48 - protein, 219 aa, Artificial Sequence
  Synthetic Construct
DIQMTQSPSSLSASVGDRVTITCRSSQSIVHSVGNTFLEWYQQKPGKAPKLLIYKVSNRFSGVPSRFSGSGSGTDFTLTISSLQPEDFATYYCFQGSQFPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 49 - protein, 450 aa, Artificial Sequence
  Synthetic Construct
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 447 aa, Artificial Sequence
  Synthetic Construct
EVQLVESGGGLVQPGGSLRLSCAASGYEFSRSWMNWVRQAPGKGLEWVGRIYPGDGDTNYSGKFKGRFTISADTSKNTAYLQMNSLRAEDTAVYYCARDGSSWDWYFDVWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 51 - protein, 219 aa, Artificial Sequence
  Synthetic Construct
DVQVTQSPSSLSASVGDRVTITCRSSQSLANSYGNTFLSWYLHKPGKAPQLLIYGISNRFSGVPDRFSGSGSGTDFTLTISSLQPEDFATYYCLQGTHQPYTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 52 - protein, 451 aa, Artificial Sequence
  Synthetic Construct
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - protein, 448 aa, Artificial Sequence
  Synthetic Construct
EVQLVQSGAEVKKPGASVKVSCKASGYRFTNYWIHWVRQAPGQGLEWIGGINPGNNYATYRRKFQGRVTMTADTSTSTVYMELSSLRSEDTAVYYCTREGYGNYGAWFAYWGQGTLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 54 - protein, 214 aa, Artificial Sequence
  Synthetic Construct
DIVMTQSPSSLSASVGDRVTITCKASQSVTNDVAWYQQKPGKAPKLLIYYASNRYTGVPSRFSGSGSGTDFTLTISSLQPEDFATYYCQQDYRSPWTFGGGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 55 - protein, 447 aa, Artificial Sequence
  Synthetic Construct
QVQLEESGGGVVRPGRSLRLSCAASGFTFDDYGVNWIRQAPGKGLEWVTIIWGDGRTDYNSALKSRFTVSRNNSNNTLSLQMNSLTTEDTAVYYCVRAPGNRAMEYWGQGVLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 56 - protein, 444 aa, Artificial Sequence
  Synthetic Construct
QVQLEESGGGVVRPGRSLRLSCAASGFTFDDYGVNWIRQAPGKGLEWVTIIWGDGRTDYNSALKSRFTVSRNNSNNTLSLQMNSLTTEDTAVYYCVRAPGNRAMEYWGQGVLVTVSSASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRVESKYGPPCPPCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 57 - protein, 20 aa, Artificial Sequence
  Synthetic Construct
EVTHQGLSSPCTKSFNRGEC

SEQ ID NO: 58 - protein, 25 aa, Artificial Sequence
  Synthetic Construct
GCLVKDYFPEPVCVSWNSGALTSGV

SEQ ID NO: 59 - protein, 25 aa, Artificial Sequence
  Synthetic Construct
LVKDYFPEPVTVCWNSGALTSGVHT

SEQ ID NO: 60 - protein, 25 aa, Artificial Sequence
  Synthetic Construct
PVTVSWNSGALTCGVHTFPAVLQSS

SEQ ID NO: 61 - protein, 25 aa, Artificial Sequence
  Synthetic Construct
SWNSGALTSGVHCFPAVLQSSGLYS

SEQ ID NO: 62 - protein, 25 aa, Artificial Sequence
  Synthetic Construct
VVTVPSSSLGTQCYICNVNHKPSNT

SEQ ID NO: 63 - protein, 25 aa, Artificial Sequence
  Synthetic Construct
VVTVPSSNFGTQCYTCNVDHKPSNT

SEQ ID NO: 64 - protein, 25 aa, Artificial Sequence
  Synthetic Construct
VVTVPSSSLGTQCYTCNVNHKPSNT

SEQ ID NO: 65 - protein, 25 aa, Artificial Sequence
  Synthetic Construct
VVTVPSSSLGTKCYTCNVDHKPSNT

SEQ ID NO: 66 - protein, 18 aa, Artificial Sequence
  Synthetic Construct
EALHNHYTQKSLCLSPGK

SEQ ID NO: 67 - protein, 18 aa, Artificial Sequence
  Synthetic Construct
EALHNRFTQKSLCLSPGK

SEQ ID NO: 68 - protein, 18 aa, Artificial Sequence
  Synthetic Construct
EALHNHYTQKSLCLSLGK

(19) United States
(12) Patent Application Publication
(10) Pub. No.: US 2016/0015831 A1
(43) Pub. Date: Jan. 21, 2016

(54) ANTI-CD22 ANTIBODY-DRUG CONJUGATES AND METHODS OF USING THEREOF

(71) Applicants: BioAlliance C.V., Alkmaar (NL); AbGenomics International Inc., Dover, DE (US)

(72) Inventors: Rong-Hwa LIN, Palo Alto, CA (US); Shih-Yao LIN, Taipei (TW); Yu-Chi HSIEH, New Taipei City (TW); Chiu-Chen HUANG, Taipei (TW); Shu-Hua LEE, Taipei (TW); Yu-Ying TSAI, Taipei City (TW); Yu-Chin LIN, Taipei (TW)

(21) Appl. No.: 14/745,184

(22) Filed: Jun. 19, 2015

Related U.S. Application Data
(60) Provisional application No. 62/015,320, filed on Jun. 20, 2014.

(51) Int. Cl. A61K 47/48; A61K 31/404; A61K 38/07; C07K 16/28; C07K 16/30

(52) U.S. Cl. CPC A61K 47/48715; A61K 47/48569; C07K 16/2851; C07K 16/30; A61K 47/48415; A61K 38/07; A61K 31/404; A61K 47/48384; C07K 2317/92; C07K 2317/73

(57) ABSTRACT

The present disclosure provides anti-CD22 antibody-drug conjugates comprising a hydrophilic self-immolative linker. The present disclosures further provide compositions and methods for treating cancers.

### CROSS-REFERENCE TO RELATED APPLICATIONS

This application claims the priority benefit of U.S. Provisional Application No. 62/015,320, filed on Jun. 20, 2014, which is hereby incorporated by reference in its entirety.

### SUBMISSION OF SEQUENCE LISTING ON ASCII TEXT FILE

The content of the following submission on ASCII text file is incorporated herein by reference in its entirety: a computer readable form (CRF) of the Sequence Listing (file name: 606592000900SEQLIST.TXT, date recorded: Jun. 17, 2015, size: 75 KB).

### FIELD OF INVENTION

The invention is in the field of cancer therapeutics, and provides efficacy and specificity for the delivery of cytotoxic drugs specifically to cancer cells through an antibody-drug conjugate (ADC) format.

### BACKGROUND

Antibody-drug conjugates (ADCs) are a class of therapeutics that combines the specificity of monoclonal antibodies (mAbs) with the potency of cytotoxic molecules. The use of ADC empowers the cancer killing activity of antibody by conjugated cytotoxic agents, while target-specific delivery avoids systemic toxicity caused by exposure to free toxic agents. As of May 2014, two ADCs have been approved by FDA for treating human cancers. Adcetris (Brentuximab vedotin or SGN-35), an anti-CD30 antibody conjugated with cytotoxic agent MMAE, is designed to treat CD30-positive relapsing lymphoma. Kadcyla (T-DM1), an anti-HER2 antibody conjugated with cytotoxic agent DM1, is designed to treat HER2-positive metastatic breast cancer.

The linker technology profoundly impacts ADC potency, specificity, and safety. Enzyme-labile linkers utilize the differential activities of proteases inside and outside of the cells to achieve control of the drug release. A drug can be conjugated to antibody via peptide bond, and can only be specifically cleaved by the action of lysosomal proteases present inside the cells, and at elevated levels in certain tumor types (Koblinski et al (2000) Clin. Chem. Acta 291:113-135). This ensures the stability of linker in the blood stream to limit the damage to healthy tissue. However, the increased hydrophobicity of some enzyme-labile linkers can lead to aggregation of ADC, particularly with strongly hydrophobic drugs. A hydrophilic self-immolative linker may provide better serum stability via specific enzyme-labile design, as well as achieve better efficacy via bystander effect on the heterogeneous cancer cells.

Numerous therapeutic agents that selectively target CD22 have been described in the treatment of hematopoietic malignancy including non-Hodgkin's lymphoma (NHL) (Sullivan-Chang et al (2013) BioDrugs 27(4):293-304) and ALL (Haso et al (2013) Blood 121(7):1165-74). Epratuzumab (hLL2 or LYMOPCIDE™ Immunomedics, Inc.), as naked antibody or radio-immunoconjugate, had demonstrated efficacy in clinical trials (Linden et al (2005) Clin Cancer Res. 11(14): 5215-22; Leonard et al (2005) J Clin Oncol 23: 5044-5051). CD22 may also be an ideal target for ADC because of its lineage-specific expression in B-cell malignancies and rapid internalization upon antibody binding. Several toxin conjugates are being developed and tested in clinical trials for treatment of CD22+ malignancies (Kantarjian et al (2012) Lancet Oncol. 13(4):403-11; Kreitman et al (2011) Clin Cancer Res. 17(20):6398-405; Kato et al (2012) Oncoimmunology. 1(9):1469-1475; Li et al (2013) Mol Cancer Ther. 12(7):1255-65; DiJoseph et al (2007) Nature Leukemia 21: 2240-2245).

There is a need for anti-cancer therapeutics having improved efficacy that can deliver cytotoxic drugs to cancer cells through an antibody-drug conjugate (ADC) format.

### SUMMARY

The compounds of the present disclosure comprise a drug moiety, a targeting moiety which is an antibody capable of targeting a selected cell population (such as a cell population expressing CD22), and a linker which contains an acyl unit, an optional spacer unit for providing distance between the drug moiety and the targeting moiety (such as an anti-CD22 antibody), a peptide linker which can be cleavable under appropriate conditions, a hydrophilic self-immolative linker, and an optional second self-immolative spacer or cyclization self-elimination linker.

The present disclosure provides a compound of the formula (I):

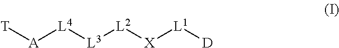

or a salt or solvate or stereoisomer thereof;

wherein:

D is a drug moiety;

T is a targeting moiety which is an antibody that specifically binds to a CD22 (e.g., a human CD22);

X is a hydrophilic self-immolative linker;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit.

The present disclosure provides a compound of the formula (II):

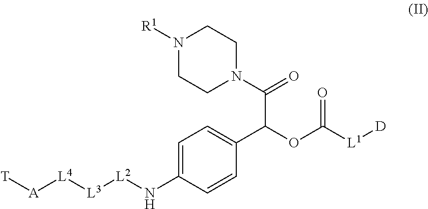

or a salt or solvate or stereoisomer thereof;

wherein:

D is a drug moiety;

T is a targeting moiety which is an antibody that specifically binds to a CD22 (e.g., a human CD22);

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit.

The present disclosure provides a compound of the formula (Ia):

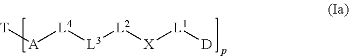

or a salt or solvate or stereoisomer thereof;

wherein:

p is 1 to 20;

D is a drug moiety;

T is a targeting moiety which is an antibody that specifically binds to a CD22 (e.g., a human CD22);

X is a hydrophilic self-immolative linker;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit.

The present disclosure provides a compound of the formula (IIa):

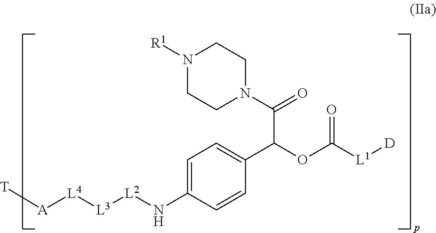

or a salt or solvate or stereoisomer thereof;

wherein:

p is 1 to 20;

D is a drug moiety;

T is a targeting moiety which is an antibody that specifically binds to a CD22 (e.g., a human CD22);

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit.

In certain embodiments of the compounds above, p is 1 to 4. In certain embodiments, L¹ is a bond. In certain embodiments, L¹ is a second self-immolative linker or a cyclization self-elimination linker. In certain embodiments, L¹ is an aminobenzyloxycarbonyl linker. In certain embodiments, L¹ is selected from the group consisting of

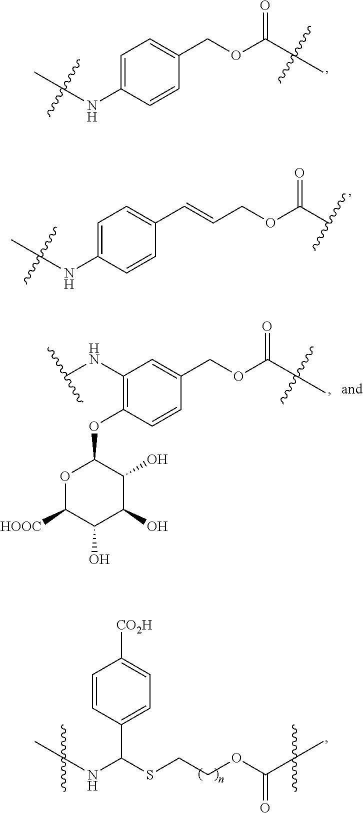

wherein n is 1 or 2.

In certain embodiments, L¹ is selected from the group consisting of

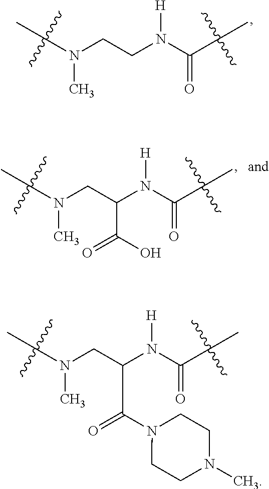

In certain embodiments of the compounds above, L² is a bond. In certain embodiments, L² is a second self-immolative linker. In certain embodiments, L² is an aminobenzyloxycarbonyl linker. In certain embodiments, L² is selected from

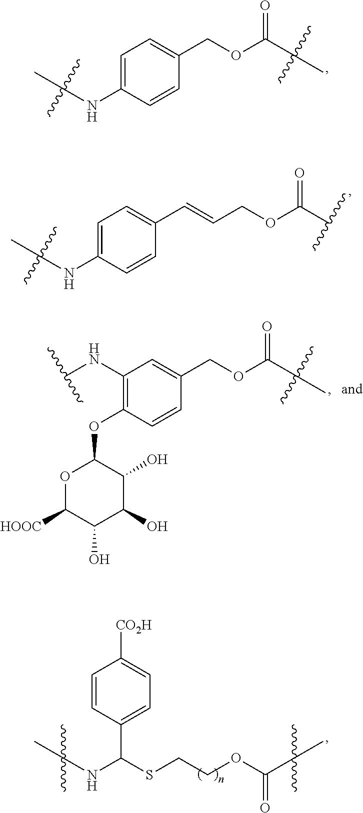

wherein n is 1 or 2.

In certain embodiments of the compounds above, L³ is a peptide linker of 1 to 10 amino acid residues. In certain embodiments, L³ is a peptide linker of 2 to 4 amino acid residues. In certain embodiments, L³ is a peptide linker comprising at least one lysine or arginine residue. In certain embodiments, L³ is a peptide linker comprising an amino acid residue selected from lysine, D-lysine, citrulline, arginine, proline, histidine, ornithine and glutamine. In certain embodiments, L³ is a peptide linker comprising an amino acid residue selected from valine, isoleucine, phenylalanine, methionine, asparagine, proline, alanine, leucine, tryptophan, and tyrosine. In certain embodiments, L³ is a dipeptide unit selected from valine-citrulline, proline-lysine, methionine-D-lysine, asparagine-D-lysine, isoleucine-proline, phenylalanine-lysine, and valine-lysine. In certain embodiments, L³ is valine-citrulline.

In certain embodiments of the compounds above, L⁴ is a bond. In certain embodiments, L⁴ is a spacer. In certain embodiments, the spacer is polyalkylene glycol, alkylene, alkenylene, alkynylene, or polyamine. In certain embodiments, L⁴ is L^{4a}-C(O), L^{4a}-C(O)—NH, L^{4a}-S(O)₂, or L^{4a}-S(O)₂—NH, wherein each L^{4a} is independently polyalkylene glycol, alkylene, alkenylene, alkynylene, or polyamine. In certain embodiments, L⁴ is L^{4a}-C(O), wherein L^{4a} is polyalkylene glycol, alkylene, alkenylene, alkynylene, or polyamine. In certain embodiments, L⁴ is L^{4a}-C(O), wherein L^{4a} is a polyalkylene glycol. In certain embodiments, L⁴ is L^{4a}-C(O), wherein L^{4a} is a polyethylene glycol. In certain embodiments, the spacer is of the formula —CH₂—(CH₂—O—CH₂)ₘ—CH₂—C(O)—, wherein m is an integer from 0 to 30. In certain embodiments, L⁴ is L^{4a}-C(O), wherein L^{4a} is alkylene.

In certain embodiments of the compounds above, A is selected from the group consisting of

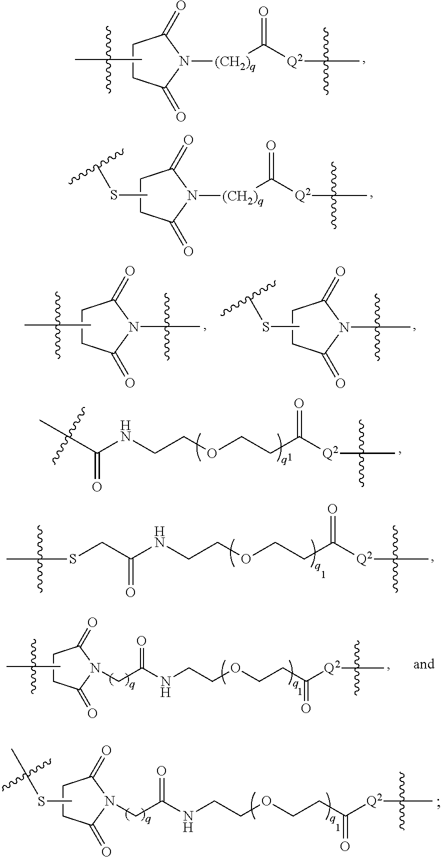

wherein each Q² is NH or O, each q is independently an integer from 1 to 10, and each q₁ is independently an integer from 1 to 10. In certain embodiments, q is 2, 3, 4, or 5. In certain embodiments, q₁ is 2, 3, 4, or 5. In certain embodiments, A is selected from the group consisting of

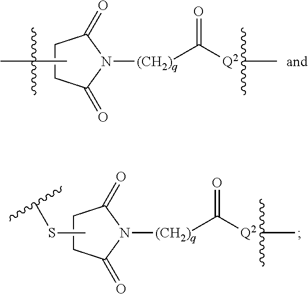

wherein each Q² is independently NH or O and each q is independently an integer from 1 to 10. In certain embodiments, q is 2, 3, 4, or 5. In certain embodiments, A is selected from the group consisting of

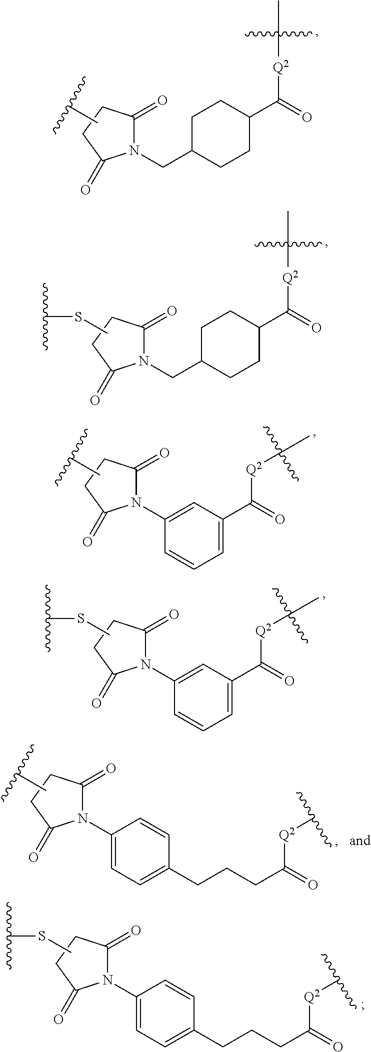

wherein each Q² is independently NH or 0.

In certain embodiments of the compounds above, one or more amino acid residues of a heavy chain of the anti-CD22 antibody are replaced with one or more cysteine residues. In certain embodiments, the antibody comprises a heavy chain constant region (e.g., a heavy chain constant region of a human IgG), wherein one or more amino acid residues in the heavy chain constant region (e.g., CH1, CH2, or CH3) are replaced with one or more cysteine residues. In certain embodiments, the antibody comprises a heavy chain constant region (e.g., a heavy chain constant region of a human IgG), wherein one or more amino acid residues selected from positions 155, 157, 165, 169, 197, and 442 in the heavy chain constant region are replaced with one or more cysteine residues, wherein the numbering is according to the EU index of Kabat. In certain embodiments, the antibody comprises a heavy chain constant region of human IgG1, human IgG2, human IgG3, human IgG4 or human IgG4p, wherein one or more amino acid residues selected from positions 155, 157, 165, 169, 197, and 442 in the heavy chain constant region are replaced with one or more cysteine residues, wherein the numbering is according to the EU index of Kabat. In certain embodiments, the antibody comprises a heavy chain constant region comprising the amino acid sequence of SEQ ID NO:12 or SEQ ID NO:13, wherein one or more amino acid residues selected from positions 155, 157, 165, 169, 197, and 442 in the heavy chain constant region are replaced with one or more cysteine residues, wherein the numbering is according to the EU index of Kabat.

In certain embodiments of the compounds above, one or more amino acid residues of a light chain of the anti-CD22 antibody are replaced with cysteine residues. In certain embodiments, the antibody comprises a light chain constant region (e.g., a human light chain kappa constant region), wherein one or more amino acid residues in the light chain constant region of the antibody are replaced with one or more cysteine residues.

In certain embodiments of the compounds above, D is linked to T by way of (or via) the added cysteine residue. In some embodiments, D is linked to T via the thiol group of the added cysteine residue connected through the linker moiety (A-L⁴-L³-L²-X-L¹-). In certain embodiments, D is an amino-containing drug moiety, wherein the drug is connected to L¹ or X through the amino group. In certain embodiments, D is duocarmycin, dolastatin, tubulysin, doxorubicin (DOX), paclitaxel, or mitomycin C (MMC), or an amino derivative thereof. In certain embodiments, D is an amino derivative of duocarmycin selected from the group consisting of

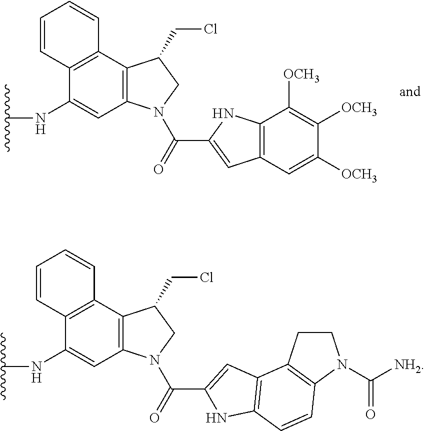 [SMILES: *Nc1cc2c(c3ccccc13)[C@H](CCl)CN2C(=O)c1cc2cc(OC)c(OC)c(OC)c2[nH]1 ; *Nc1cc2c(c3ccccc13)[C@H](CCl)CN2C(=O)c1cc2c3c(ccc2[nH]1)N(C(N)=O)CC3]

In certain embodiments, D is an amino derivative of dolastatin (e.g. monomethyl Dolastatin 10):

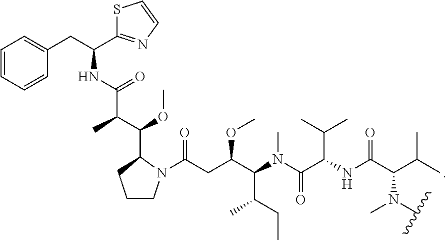 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C)C(C)C]

In certain embodiments, -A-L⁴-L³-L²- is

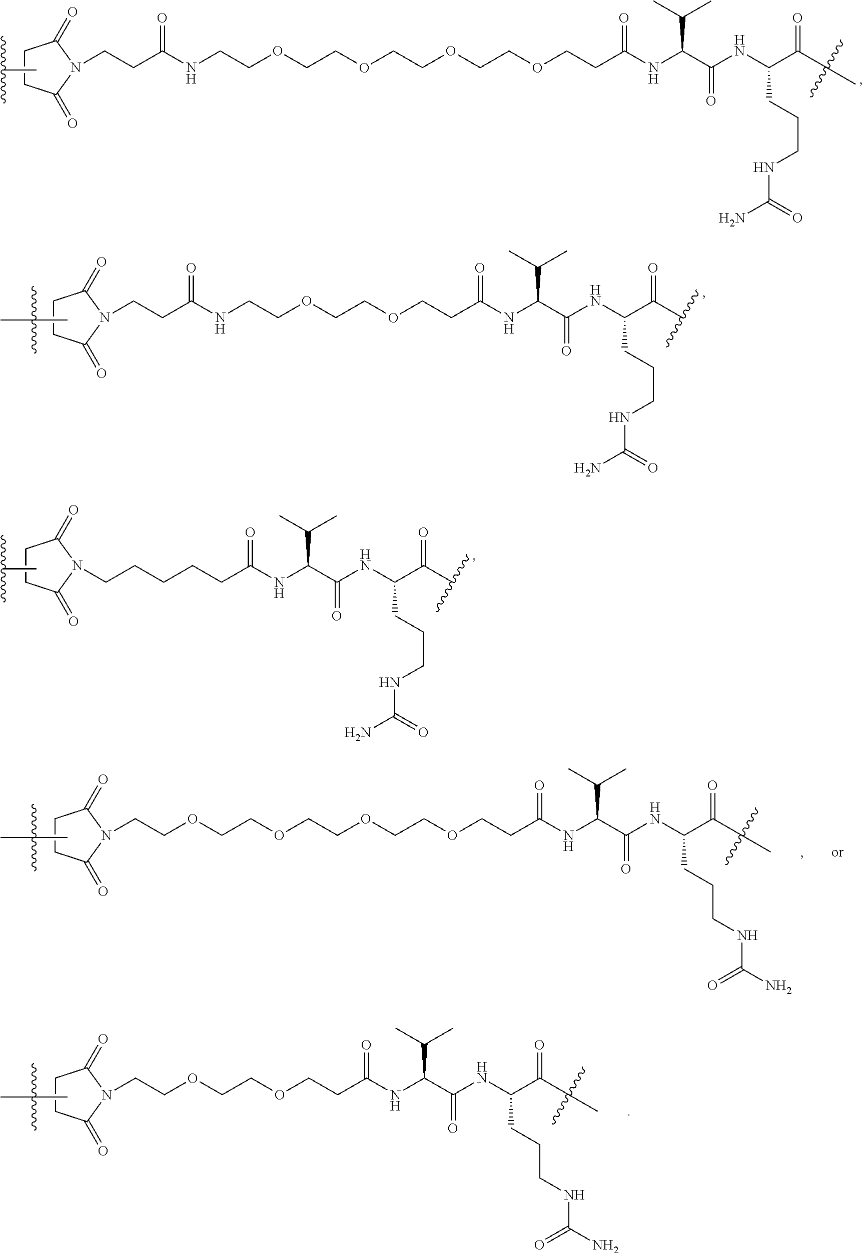

In certain embodiments, -A-L⁴-L³-L²-X-L¹-D is:

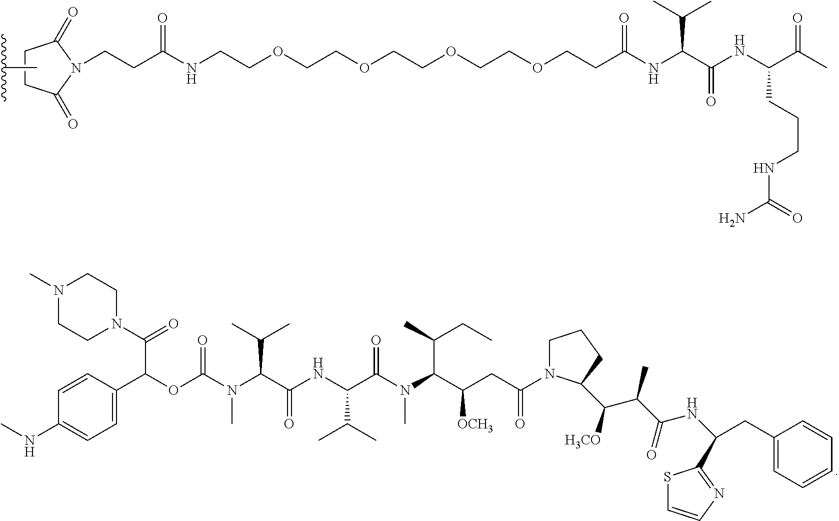

In certain embodiments, -A-L⁴-L³-L²-X-L¹-D is:

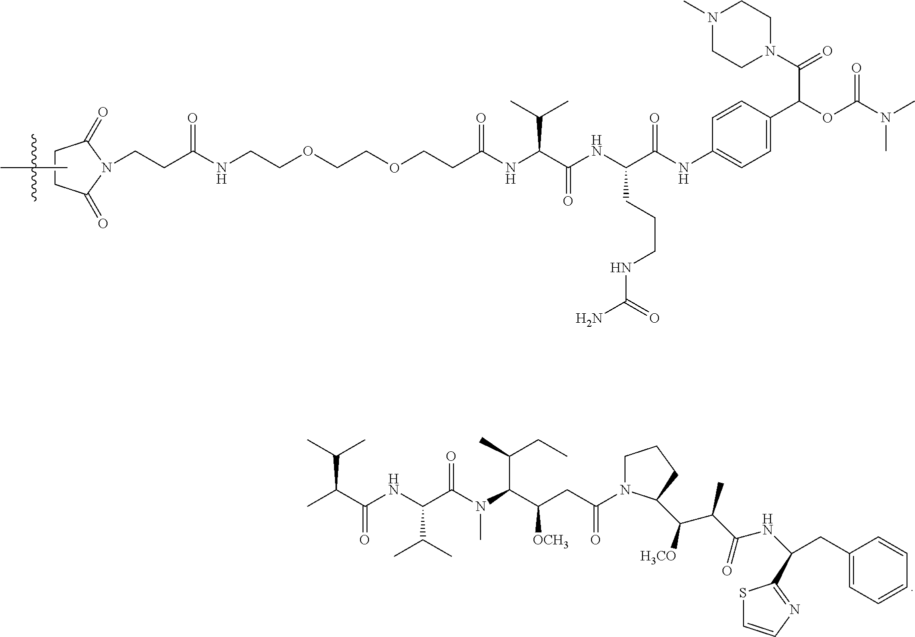

In certain embodiments, -A-L⁴-L³-L²-X-L¹-D is:

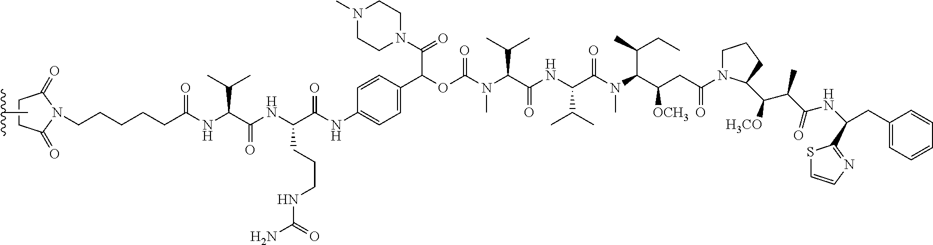

In certain embodiments of the compounds above, the anti-CD22 antibody is a humanized antibody, a chimeric antibody or a human antibody.

In certain embodiments, the anti-CD22 antibody comprises a heavy chain variable region and a light chain variable region, wherein:

(1) the heavy chain variable region comprises the three heavy chain HVRs (e.g., SEQ ID NOs: 15, 16, and 17) of the amino acid sequence of SEQ ID NO:2 and/or the light chain variable region comprises the three light chain HVRs (e.g., SEQ ID NOs: 18, 19, and 20) of the amino acid sequence of SEQ ID NO:1;

(2) the heavy chain variable region comprises the three heavy chain HVRs (e.g., SEQ ID NOs: 21, 22, and 23) of the amino acid sequence of SEQ ID NO:4 and/or the light chain variable region comprises the three light chain HVRs (e.g., SEQ ID NOs: 24, 25, and 26) of the amino acid sequence of SEQ ID NO:3;

(3) the heavy chain variable region comprises the three heavy chain HVRs (e.g., SEQ ID NOs: 27, 28, and 29) of the amino acid sequence of SEQ ID NO:6 and/or the light chain variable region comprises the three light chain HVRs (e.g., SEQ ID NOs: 30, 31, and 32) of the amino acid sequence of SEQ ID NO:5;

(4) the heavy chain variable region comprises the three heavy chain HVRs (e.g., SEQ ID NOs: 33, 34, and 35) of the amino acid sequence of SEQ ID NO:8 and/or the light chain variable region comprises the three light chain HVRs (e.g., SEQ ID NOs: 36, 37, and 38) of the amino acid sequence of SEQ ID NO:7; or

(5) the heavy chain variable region comprises the three heavy chain HVRs (e.g., SEQ ID NOs: 39, 40, and 41) of the amino acid sequence of SEQ ID NO:10 and/or the light chain variable region comprises the three light chain HVRs (e.g., SEQ ID NOs: 42, 43, and 44) of the amino acid sequence of SEQ ID NO:9.

In certain embodiments, the anti-CD22 antibody comprises a heavy chain variable region and a light chain variable region, wherein

(1) the heavy chain variable region comprises the amino acid sequence of SEQ ID NO:2 and/or the light chain variable region comprises the amino acid sequence of SEQ ID NO:1;

(2) the heavy chain variable region comprises the amino acid sequence of SEQ ID NO:4 and/or the light chain variable region comprises the amino acid sequence of SEQ ID NO:3;

(3) the heavy chain variable region comprises the amino acid sequence of SEQ ID NO:6 and/or the light chain variable region comprises the amino acid sequence of SEQ ID NO:5;

(4) the heavy chain variable region comprises the amino acid sequence of SEQ ID NO:8 and/or the light chain variable region comprises the amino acid sequence of SEQ ID NO:7;

or

(5) the heavy chain variable region comprises the amino acid sequence of SEQ ID NO:10 and/or the light chain variable region comprises the amino acid sequence of SEQ ID NO:9.

In certain embodiments, the anti-CD22 antibody comprises a heavy chain and a light chain, wherein

(1) the heavy chain comprises the amino acid sequence of SEQ ID NO:46 and/or the light chain comprises the amino acid sequence of SEQ ID NO:45;

(2) the heavy chain comprises the amino acid sequence of SEQ ID NO:47 and/or the light chain comprises the amino acid sequence of SEQ ID NO:45;

(3) the heavy chain comprises the amino acid sequence of SEQ ID NO:49 and/or the light chain comprises the amino acid sequence of SEQ ID NO:48;

(4) the heavy chain comprises the amino acid sequence of SEQ ID NO:50 and/or the light chain comprises the amino acid sequence of SEQ ID NO:48;

(5) the heavy chain comprises the amino acid sequence of SEQ ID NO:52 and/or the light chain comprises the amino acid sequence of SEQ ID NO:51;

(6) the heavy chain comprises the amino acid sequence of SEQ ID NO:53 and/or the light chain comprises the amino acid sequence of SEQ ID NO:51;

(7) the heavy chain comprises the amino acid sequence of SEQ ID NO:55 and/or the light chain comprises the amino acid sequence of SEQ ID NO:54; or

(8) the heavy chain comprises the amino acid sequence of SEQ ID NO:56 and/or the light chain comprises the amino acid sequence of SEQ ID NO:54; or

(9) the heavy chain comprises a variable region comprising the amino acid sequence of SEQ ID NO:10 and a constant region comprising the amino acid sequence of SEQ ID NO: 12 and/or the light chain comprises a variable region comprising the amino acid sequence of SEQ ID NO:9 and a constant region comprising the amino acid sequence of SEQ ID NO: 11.

(10) the heavy chain comprises a variable region comprising the amino acid sequence of SEQ ID NO:10 and a constant region comprising the amino acid sequence of SEQ ID NO: 13 and/or the light chain comprises a variable region comprising the amino acid sequence of SEQ ID NO:9 and a constant region comprising the amino acid sequence of SEQ ID NO: 11.

In certain embodiments, the antibody comprises a human heavy chain constant region comprising the amino acid sequence of SEQ ID NO:12 or SEQ ID NO:13 and a human light chain kappa constant region comprising the amino acid sequence of SEQ ID NO:11, wherein one or more amino acid residues selected from T155, S157, S165, T169, T197, and S442 in the heavy chain constant region are replaced with a cysteine residue, and wherein the numbering is according to the EU index of Kabat. In some of these embodiments, at least one (e.g., one) amino acid residue selected from T155, S157, S165, T169, T197, and S442 in the heavy chain constant region is replaced with a cysteine residue. In certain embodiments, the antibody is an antigen-binding fragment selected from the group consisting of Fab, Fab′, F(ab′)₂, Fv, and ScFv.

The present disclosure provides a pharmaceutical composition comprising a compound described above and herein, or a salt or solvate or stereoisomer thereof; and a pharmaceutically acceptable carrier.

The present disclosure provides a method of killing a cell that expresses a human CD22, comprising administering to the cell an amount of a compound described herein, or a salt or solvate or stereoisomer thereof, sufficient to kill the cell. In certain embodiments, the cell is a cancer cell. In certain embodiments, the cancer cell is in an individual (e.g., a human). In certain embodiments, the cancer cell is a CD22-positive lymphoma or leukemia cell.

The present disclosure provides a method of treating cancer in an individual comprising administering to the individual an effective amount of a compound described herein, or a salt or solvate or stereoisomer thereof. In certain embodiments, the individual has cancer or has been diagnosed with cancer. In certain embodiments, the cancer is a CD22-positive hematological malignancy. In certain embodiments, the CD22-positive hematological malignancy is a B cell lymphoma or acute lymphoblastic leukemia. In certain embodiments, the individual is a human.

The present disclosure provides a kit comprising a compound described herein, or a salt or solvate or stereoisomer thereof. In certain embodiments, the kit further comprises instructions for use in the treatment of cancer.

Provided herein is a process for making a compound of formula (II):

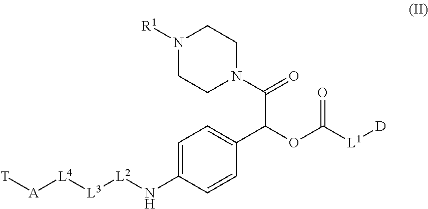

or a salt or solvate or stereoisomer thereof;

wherein:

D is a drug moiety;

T is a targeting moiety which is an antibody that specifically binds to a CD22 (e.g., a human CD22);

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit;

comprising reacting the antibody with Compound Z:

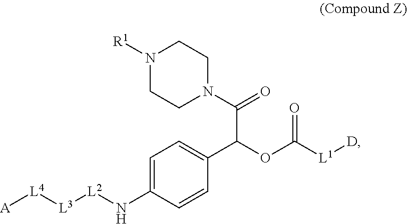

or a salt or solvate or stereoisomer thereof.

Provided herein is a process for making a compound of formula (IIa):

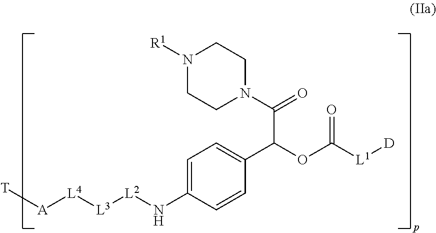

or a salt or solvate or stereoisomer thereof;

wherein:

p is 1 to 20;

D is a drug moiety;

T is a targeting moiety which is an antibody that specifically binds to a CD22 (e.g., a human CD22);

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit;

comprising reacting the antibody with Compound Z:

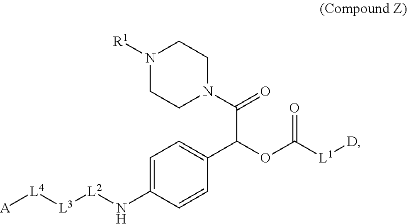

or a salt or solvate or stereoisomer thereof.

In certain embodiments of the methods and processes herein, the antibody comprises one or more sulfhydryl groups. In certain embodiments, one or more amino acid residues of a heavy chain of the anti-CD22 antibody are replaced with one or more cysteine residues. In certain embodiments, one or more amino acid residues in the heavy chain constant region (e.g., CH1, CH2, or CH3) are replaced with one or more cysteine residues. In certain embodiments, the antibody comprises a heavy chain constant region (e.g., a heavy chain constant region of a human IgG), wherein one or more amino acid residues selected from positions 155, 157, 165, 169, 197, and 442 in the heavy chain constant region are replaced with one or more cysteine residues, wherein the numbering is according to the EU index of Kabat. In certain embodiments, the antibody comprises a heavy chain constant region of human IgG1, human IgG2, human IgG3, human IgG4 or human IgG4p, wherein one or more amino acid residues selected from positions 155, 157, 165, 169, 197, and 442 in the heavy chain constant region are replaced with one or more cysteine residues, wherein the numbering is according to the EU index of Kabat. In certain embodiments, the antibody comprises a heavy chain constant region comprising the amino acid sequence of SEQ ID NO:12 or SEQ ID NO:13, wherein one or more amino acid residues selected from positions 155, 157, 165, 169, 197, and 442 in the heavy chain constant region are replaced with one or more cysteine residues, wherein the numbering is according to the EU index of Kabat.

In certain embodiments of the methods and processes herein, one or more amino acid residues of a light chain of the antibody are replaced with one or more cysteine residues. In certain embodiments, one or more amino acid residues in the light chain constant region of the antibody are replaced with one or more cysteine residues.

In certain embodiments of the methods or processes herein, D is linked to T by way of (or via) the added cysteine residue. In some embodiments, D is linked to T via the thiol group of the added cysteine residue connected through the linker moiety (-A-L⁴-L³-L²-X-L¹-).

In certain embodiments of the methods or processes herein, the anti-CD22 antibody is a humanized antibody, a chimeric antibody or a human antibody. In certain embodiments, the anti-CD22 antibody comprises a heavy chain variable region and a light chain variable region, wherein:

(1) the heavy chain variable region comprises the three heavy chain HVRs (e.g., SEQ ID NOs: 15, 16, and 17) of the amino acid sequence of SEQ ID NO:2 and/or the light chain variable region comprises the three light chain HVRs (e.g., SEQ ID NOs: 18, 19, and 20) of the amino acid sequence of SEQ ID NO:1;

(2) the heavy chain variable region comprises the three heavy chain HVRs (e.g., SEQ ID NOs: 21, 22, and 23) of the amino acid sequence of SEQ ID NO:4 and/or the light chain variable region comprises the three light chain HVRs (e.g., SEQ ID NOs: 24, 25, and 26) of the amino acid sequence of SEQ ID NO:3;

(3) the heavy chain variable region comprises the three heavy chain HVRs (e.g., SEQ ID NOs: 27, 28, and 29) of the amino acid sequence of SEQ ID NO:6 and/or the light chain variable region comprises the three light chain HVRs (e.g., SEQ ID NOs: 30, 31, and 32) of the amino acid sequence of SEQ ID NO:5;

(4) the heavy chain variable region comprises the three heavy chain HVRs (e.g., SEQ ID NOs: 33, 34, and 35) of the amino acid sequence of SEQ ID NO:8 and/or the light chain variable region comprises the three light chain HVRs (e.g., SEQ ID NOs: 36, 37, and 38) of the amino acid sequence of SEQ ID NO:7; or

(5) the heavy chain variable region comprises the three heavy chain HVRs (e.g., SEQ ID NOs: 39, 40, and 41) of the amino acid sequence of SEQ ID NO:10 and/or the light chain variable region comprises the three light chain HVRs (e.g., SEQ ID NOs: 42, 43, and 44) of the amino acid sequence of SEQ ID NO:9.

In certain embodiments of the methods and processes herein, the anti-CD22 antibody comprises a heavy chain variable region and a light chain variable region, wherein

(1) the heavy chain variable region comprises the amino acid sequence of SEQ ID NO:2 and/or the light chain variable region comprises the amino acid sequence of SEQ ID NO:1;

(2) the heavy chain variable region comprises the amino acid sequence of SEQ ID NO:4 and/or the light chain variable region comprises the amino acid sequence of SEQ ID NO:3;

(3) the heavy chain variable region comprises the amino acid sequence of SEQ ID NO:6 and/or the light chain variable region comprises the amino acid sequence of SEQ ID NO:5;

(4) the heavy chain variable region comprises the amino acid sequence of SEQ ID NO:8 and/or the light chain variable region comprises the amino acid sequence of SEQ ID NO:7;

or

(5) the heavy chain variable region comprises the amino acid sequence of SEQ ID NO:10 and/or the light chain variable region comprises the amino acid sequence of SEQ ID NO:9.

In certain embodiments, the anti-CD22 antibody comprises a heavy chain and a light chain, wherein

(1) the heavy chain comprises the amino acid sequence of SEQ ID NO:46 and/or the light chain comprises the amino acid sequence of SEQ ID NO:45;

(2) the heavy chain comprises the amino acid sequence of SEQ ID NO:47 and/or the light chain comprises the amino acid sequence of SEQ ID NO:45;

(3) the heavy chain comprises the amino acid sequence of SEQ ID NO:49 and/or the light chain comprises the amino acid sequence of SEQ ID NO:48;

(4) the heavy chain comprises the amino acid sequence of SEQ ID NO:50 and/or the light chain comprises the amino acid sequence of SEQ ID NO:48;

(5) the heavy chain comprises the amino acid sequence of SEQ ID NO:52 and/or the light chain comprises the amino acid sequence of SEQ ID NO:51;

(6) the heavy chain comprises the amino acid sequence of SEQ ID NO:53 and/or the light chain comprises the amino acid sequence of SEQ ID NO:51;

(7) the heavy chain comprises the amino acid sequence of SEQ ID NO:55 and/or the light chain comprises the amino acid sequence of SEQ ID NO:54; or

(8) the heavy chain comprises the amino acid sequence of SEQ ID NO:56 and/or the light chain comprises the amino acid sequence of SEQ ID NO:54; or

(9) the heavy chain comprises a variable region comprising the amino acid sequence of SEQ ID NO:10 and a constant region comprising the amino acid sequence of SEQ ID NO: 12 and/or the light chain comprises a variable region comprising the amino acid sequence of SEQ ID NO:9 and a constant region comprising the amino acid sequence of SEQ ID NO: 11.

(10) the heavy chain comprises a variable region comprising the amino acid sequence of SEQ ID NO:10 and a constant region comprising the amino acid sequence of SEQ ID NO: 13 and/or the light chain comprises a variable region comprising the amino acid sequence of SEQ ID NO:9 and a constant region comprising the amino acid sequence of SEQ ID NO: 11.

In certain embodiments of the methods and processes herein, the antibody comprises a heavy chain constant region comprising the amino acid sequence of SEQ ID NO:12 or SEQ ID NO:13 and a human light chain kappa constant region comprising the amino acid sequence of SEQ ID NO:11, wherein one or more amino acid residues selected from T155, S157, S165, T169, T197, and S442 in the heavy chain constant region are replaced with a cysteine residue, wherein the numbering is according to the EU index of Kabat. In some of these embodiments, at least one (e.g., one) amino acid residue selected from T155, S157, S165, T169, T197, and S442 in the heavy chain constant region is replaced with a cysteine residue.

The present disclosure provides a compound, or a salt or solvate or stereoisomer thereof, wherein the compound is prepared by a method or process described herein, wherein the antibody comprises one or more sulfhydryl groups.

The present disclosure provides a pharmaceutical composition comprising a compound, or a salt or solvate or stereoisomer thereof, wherein the compound is prepared by a process described herein, wherein the antibody comprises one or more sulfhydryl groups, and a pharmaceutically acceptable carrier.

It is to be understood that one, some, or all of the properties of the various embodiments described herein may be combined to form other embodiments of the present invention. These and other aspects of the invention will become apparent to one of skill in the art.

### BRIEF DESCRIPTION OF THE DRAWINGS

FIG. 1 shows an NMR spectrum of Tap-18H.

FIG. 2 shows an NMR spectrum of Tap-18Hr1.

FIG. 3 shows an NMR spectrum of Tap-18Hr2.

FIG. 4 shows in vivo anti-tumor activity of Anti-CD22 (hLL2)-IgG1-Tap18Hr1 against xenograft derived from B-cell lymphoma cell line Daudi.

FIG. 5 shows in vivo anti-tumor activity of Anti-CD22 (hLL2)-IgG1-Tap18Hr1 against xenograft derived from B-cell lymphoma cell line Ramos.

FIG. 6 shows in vivo anti-tumor activity of the site-specific conjugated Anti-CD22-Tap18Hr1 ADCs hLL2-S157C-IgG1-Tap18Hr1, hLL2-S442C-IgG1-Tap18Hr1, hLL2-T155C-IgG4p-Tap18Hr1, hLL2-T169C-IgG4p-Tap18Hr1, and hLL2-S442C-IgG4p-Tap18Hr1 against xenograft derived from B-cell lymphoma cell line Ramos.

FIG. 7 shows in vivo anti-tumor activity of the site-specific conjugated Anti-CD22-Tap18Hr1 ADCs (hLL2-S157C-IgG1-Tap18Hr1, hLL2-T155C-IgG4p-Tap18Hr1, hLL2-T169C-IgG4p-Tap18Hr1, and hLL2-S442C-IgG4p-Tap18Hr1 against xenograft derived from acute lymphoblastic leukemia cell line REH.

### DEFINITIONS

The following terms have the following meanings unless otherwise indicated. Any undefined terms have their art recognized meanings.

“Alkyl” refers to monovalent saturated aliphatic hydrocarbyl groups having from 1 to 10 carbon atoms and preferably 1 to 6 carbon atoms. This term includes, by way of example, linear and branched hydrocarbyl groups such as methyl (CH₃—), ethyl (CH₃CH₂—), n-propyl (CH₃CH₂CH₂—), isopropyl ((CH₃)₂CH—), n-butyl (CH₃CH₂CH₂CH₂—), isobutyl ((CH₃)₂CHCH₂—), sec-butyl ((CH₃)(CH₃CH₂)CH—), t-butyl ((CH₃)₃C—), n-pentyl (CH₃CH₂CH₂CH₂CH₂—), neopentyl ((CH₃)₃CCH₂—), and n-hexyl (CH₃(CH₂)₅—).

“Alkylene” refers to divalent aliphatic hydrocarbylene groups preferably having from 1 to 10 and more preferably 1 to 3 carbon atoms that are either straight-chained or branched. This term includes, by way of example, methylene (—CH₂—), ethylene (—CH₂CH₂—), n-propylene (—CH₂CH₂CH₂—), iso-propylene (—CH₂CH(CH₃)—), (—C(CH₃)₂CH₂CH₂—), (—C(CH₃)₂CH₂C(O)—), (—C(CH₃)₂CH₂C(O)NH—), (—CH(CH₃)CH₂—), and the like.

“Alkenyl” refers to straight chain or branched hydrocarbyl groups having from 2 to 10 carbon atoms and preferably 2 to 4 carbon atoms and having at least 1 and preferably from 1 to 2 sites of double bond unsaturation. This term includes, by way of example, bi-vinyl, allyl, and but-3-en-1-yl. Included within this term are the cis and trans isomers or mixtures of these isomers.

“Alkenylene” refers to straight chain or branched hydrocarbylene groups having from 2 to 10 carbon atoms and preferably 2 to 4 carbon atoms and having at least 1 and preferably from 1 to 2 sites of double bond unsaturation. Examples of alkenylene include, but is not limited to, vinylene (—CH═CH—), allylene (—CH₂C═C—), and but-3-en-1-ylene (—CH₂CH₂C═CH—). Included within this term are the cis and trans isomers or mixtures of these isomers.

“Alkynyl” refers to straight or branched hydrocarbyl groups having from 2 to 6 carbon atoms and preferably 2 to 3 carbon atoms and having at least 1 and preferably from 1 to 2 sites of triple bond unsaturation. Examples of such alkynyl groups include acetylenyl (—C≡CH), and propargyl (—CH₂C≡CH).

“Alkynylene” refers to straight or branched hydrocarbylene groups having from 2 to 6 carbon atoms and preferably 2 to 3 carbon atoms and having at least 1 and preferably from 1 to 2 sites of triple bond unsaturation. Examples of alkynylene include, but are not limited to, acetylenylene (—C≡C—), and propargylene (—CH₂C≡C—).

“Amino” refers to the group —NH₂.

“Substituted amino” refers to the group —NRR where each R is independently selected from the group consisting of hydrogen, alkyl, substituted alkyl, cycloalkyl, substituted cycloalkyl, alkenyl, substituted alkenyl, cycloalkenyl, substituted cycloalkenyl, alkynyl, substituted alkynyl, aryl, heteroaryl, and heterocyclyl provided that at least one R is not hydrogen.

“Aryl” refers to a monovalent aromatic carbocyclic group of from 6 to 18 carbon atoms having a single ring (such as is present in a phenyl group) or a ring system having multiple condensed rings (examples of such aromatic ring systems include naphthyl, anthryl and indanyl) which condensed rings may or may not be aromatic, provided that the point of attachment is through an atom of an aromatic ring. This term includes, by way of example, phenyl and naphthyl. Unless otherwise constrained by the definition for the aryl substituent, such aryl groups can optionally be substituted with from 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxyl ester, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, sulfonylamino, —SO-alkyl, —SO-substituted alkyl, —SO-aryl, —SO-heteroaryl, —SO₂-alkyl, —SO₂-substituted alkyl, —SO₂-aryl, —SO₂-heteroaryl and trihalomethyl.

“Cycloalkyl” refers to cyclic alkyl groups of from 3 to 10 carbon atoms having single or multiple cyclic rings including fused, bridged, and spiro ring systems. Examples of suitable cycloalkyl groups include, for instance, adamantyl, cyclopropyl, cyclobutyl, cyclopentyl, cyclooctyl and the like. Such cycloalkyl groups include, by way of example, single ring structures such as cyclopropyl, cyclobutyl, cyclopentyl, cyclooctyl, and the like, or multiple ring structures such as adamantyl, and the like.

“Heteroaryl” refers to an aromatic group of from 1 to 15 carbon atoms, such as from 1 to 10 carbon atoms and 1 to 10 heteroatoms selected from the group consisting of oxygen, nitrogen, and sulfur within the ring. Such heteroaryl groups can have a single ring (such as, pyridinyl, imidazolyl or furyl) or multiple condensed rings in a ring system (for example as in groups such as, indolizinyl, quinolinyl, benzofuranyl, benzimidazolyl or benzothienyl), wherein at least one ring within the ring system is aromatic and at least one ring within the ring system is aromatic, provided that the point of attachment is through an atom of an aromatic ring. In certain embodiments, the nitrogen and/or sulfur ring atom(s) of the heteroaryl group are optionally oxidized to provide for the N-oxide (N→O), sulfinyl, or sulfonyl moieties. This term includes, by way of example, pyridinyl, pyrrolyl, indolyl, thiophenyl, and furanyl. Unless otherwise constrained by the definition for the heteroaryl substituent, such heteroaryl groups can be optionally substituted with 1 to 5 substituents, or from 1 to 3 substituents, selected from acyloxy, hydroxy, thiol, acyl, alkyl, alkoxy, alkenyl, alkynyl, cycloalkyl, cycloalkenyl, substituted alkyl, substituted alkoxy, substituted alkenyl, substituted alkynyl, substituted cycloalkyl, substituted cycloalkenyl, amino, substituted amino, aminoacyl, acylamino, alkaryl, aryl, aryloxy, azido, carboxyl, carboxyl ester, cyano, halogen, nitro, heteroaryl, heteroaryloxy, heterocyclyl, heterocyclooxy, aminoacyloxy, oxyacylamino, thioalkoxy, substituted thioalkoxy, thioaryloxy, thioheteroaryloxy, sulfonylamino, —SO-alkyl, —SO-substituted alkyl, —SO-aryl, —SO— heteroaryl, —SO₂-alkyl, —SO₂-substituted alkyl, —SO₂-aryl and —SO₂-heteroaryl, and trihalomethyl.

Examples of heteroaryls include, but are not limited to, pyrrole, imidazole, pyrazole, pyridine, pyrazine, pyrimidine, pyridazine, indolizine, isoindole, indole, purine, isoquinoline, quinoline, phthalazine, naphthyridine, quinoxaline, quinazoline, cinnoline, pteridine, carbazole, carboline, phenanthridine, acridine, phenanthroline, isothiazole, phenazine, isoxazole, phenoxazine, phenothiazine, piperidine, piperazine, phthalimide, 4,5,6,7-tetrahydrobenzo[b]thiophene, thiazole, thiophene, benzo[b]thiophene, and the like.

“Heterocycle,” “heterocyclic,” “heterocycloalkyl” or “heterocyclyl” refers to a saturated or partially unsaturated group having a single ring or multiple condensed rings, including fused, bridged, or spiro ring systems, and having from 3 to 20 ring atoms, including 1 to 10 hetero atoms. These ring atoms are selected from the group consisting of carbon, nitrogen, sulfur, or oxygen, wherein, in fused ring systems, one or more of the rings can be cycloalkyl, aryl, or heteroaryl, provided that the point of attachment is through the non-aromatic ring. In certain embodiments, the nitrogen and/or sulfur atom(s) of the heterocyclic group are optionally oxidized to provide for N-oxide, —S(O)—, or —SO₂— moieties.

Examples of heterocycles include, but are not limited to, azetidine, dihydroindole, indazole, quinolizine, imidazolidine, imidazoline, piperidine, piperazine, indoline, 1,2,3,4-tetrahydroisoquinoline, thiazolidine, morpholinyl, thiomorpholinyl (also referred to as thiamorpholinyl), 1,1-dioxothiomorpholinyl, piperidinyl, pyrrolidine, tetrahydrofuranyl, and the like.

Where a heteroaryl or heterocyclyl group is “substituted,” unless otherwise constrained by the definition for the heteroaryl or heterocyclic substituent, such heteroaryl or heterocyclic groups can be substituted with 1 to 5, or from 1 to 3 substituents, selected from alkyl, substituted alkyl, alkoxy, substituted alkoxy, cycloalkyl, substituted cycloalkyl, cycloalkenyl, substituted cycloalkenyl, acyl, acylamino, acyloxy, amino, substituted amino, aminoacyl, aminoacyloxy, azido, cyano, halogen, hydroxyl, oxo, thioketo, carboxyl, carboxyl ester, thioaryloxy, thioheteroaryloxy, thioheterocyclooxy, thiol, thioalkoxy, substituted thioalkoxy, aryl, aryloxy, heteroaryl, heteroaryloxy, heterocyclyl, heterocyclooxy, hydroxyamino, alkoxyamino, nitro, sulfonylamino, —SO-alkyl, —SO-substituted alkyl, —SO-aryl, —SO-heteroaryl, —SO— heterocyclyl, —SO₂-alkyl, —SO₂-substituted alkyl, —SO₂-aryl, —SO₂-heteroaryl, and —SO₂-heterocyclyl.

“Polyalkylene glycol” refers to straight or branched polyalkylene glycol polymers such as polyethylene glycol, polypropylene glycol, and polybutylene glycol. A polyalkylene glycol subunit is a single polyalkylene glycol unit. For example, an example of a polyethylene glycol subunit would be an ethylene glycol, —O—CH₂—CH₂—O—, or propylene glycol, —O—CH₂—CH₂—CH₂—O—, capped with a hydrogen at the chain termination point. Other examples of poly(alkylene glycol) include, but are not limited to, PEG, PEG derivatives such as methoxypoly(ethylene glycol) (mPEG), poly(ethylene oxide), PPG, poly(tetramethylene glycol), poly(ethylene oxide-co-propylene oxide), or copolymers and combinations thereof.

“Polyamine” refers to polymers having an amine functionality in the monomer unit, either incorporated into the backbone, as in polyalkyleneimines, or in a pendant group as in polyvinyl amines.

In addition to the disclosure herein, the term “substituted,” when used to modify a specified group or radical, can also mean that one or more hydrogen atoms of the specified group or radical are each, independently of one another, replaced with the same or different substituent groups as defined below.

In addition to the groups disclosed with respect to the individual terms herein, substituent groups for substituting for one or more hydrogens (any two hydrogens on a single carbon can be replaced with ═O, ═NR⁷⁰, ═N—OR⁷⁰, ═N₂ or ═S) on saturated carbon atoms in the specified group or radical are, unless otherwise specified, —R⁶⁰, halo, ═O, —OR⁷⁰, —SR⁷⁰, —NR⁸⁰R⁸⁰, trihalomethyl, —CN, —OCN, —SCN, —NO, —NO₂, ═N₂, —N₃, —S(O)R⁷⁰, —S(O)₂R⁷⁰, —SO₃⁻M⁺; —S(O)₂OR⁷⁰, —OS(O)₂R⁷⁰, —OS(O)₂OR⁷⁰, —PO₃²⁻(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —C(O)O⁻M⁺, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)O⁻M⁺, —OC(O)OR⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰ is selected from the group consisting of optionally substituted alkyl, cycloalkyl, heterocycloalkyl, heterocycloalkylalkyl, cycloalkylalkyl, aryl, arylalkyl, heteroaryl and heteroarylalkyl, each R⁷⁰ is independently hydrogen or R⁶⁰; each R⁸⁰ is independently R⁷⁰ or alternatively, two R⁸⁰'s, taken together with the nitrogen atom to which they are bonded, form a 3-, 4-, 5-, 6-, or 7-membered heterocycloalkyl which may optionally include from 1 to 4 of the same or different additional heteroatoms selected from the group consisting of O, N and S, of which N may have —H, C₁-C₄ alkyl, —C(O)C₁₋₄alkyl, —CO₂C₁₋₄alkyl, or —S(O)₂C₁₋₄alkyl substitution; and each M⁺ is a counter ion with a net single positive charge. Each M⁺ may independently be, for example, an alkali ion, such as K⁺, Na⁺, Li⁺; an ammonium ion, such as ⁺N(R⁶⁰)₄; or an alkaline earth ion, such as [Ca²⁺]_{0.5}, [Mg²⁺]_{0.5}, or [Ba²⁺]_{0.5} (“subscript 0.5 means that one of the counter ions for such divalent alkali earth ions can be an ionized form of a compound of the embodiments and the other a typical counter ion such as chloride, or two ionized compounds disclosed herein can serve as counter ions for such divalent alkali earth ions, or a doubly ionized compound of the embodiments can serve as the counter ion for such divalent alkali earth ions).

In addition to the disclosure herein, substituent groups for hydrogens on unsaturated carbon atoms in “substituted” alkene, alkyne, aryl and heteroaryl groups are, unless otherwise specified, —R⁶⁰, halo, —O⁻M⁺, —OR⁷⁰, —SR⁷⁰, —S⁻M⁺, —NR⁸⁰R⁸⁰, trihalomethyl, —CF₃, —CN, —OCN, —SCN, —NO, —NO₂, —N₃, —S(O)R⁷⁰, —S(O)₂R⁷⁰, —SO₃⁻M⁺, —SO₃R⁷⁰, —OS(O)₂R⁷⁰, —OSO₃⁻M⁺, —OSO₃R⁷⁰, —PO₃²⁻(M⁺)₂, —P(O)(OR⁷⁰)O⁻M⁺, —P(O)(OR⁷⁰)₂, —C(O)R⁷⁰, —C(S)R⁷⁰, —C(NR⁷⁰)R⁷⁰, —CO₂⁻M⁺, —CO₂R⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OCO₂⁻M⁺, —OCO₂R⁷⁰, —OC(S)OR⁷⁰, —NR⁷⁰C(O)R⁷⁰, —NR⁷⁰C(S)R⁷⁰, —NR⁷⁰CO₂⁻M⁺, —NR⁷⁰CO₂R⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined, provided that in case of substituted alkene or alkyne, the substituents are not —O⁻M⁺, —OR⁷⁰, —SR⁷⁰, or —S⁻M⁺.

In addition to the substituent groups disclosed with respect to the individual terms herein, substituent groups for hydrogens on nitrogen atoms in “substituted” heterocycloalkyl and cycloalkyl groups are, unless otherwise specified, —R⁶⁰, —O⁻M⁺, —OR⁷⁰, —SR⁷⁰, —S⁻M⁺, —NR⁸⁰R⁸⁰, trihalomethyl, —CF₃, —CN, —NO, —NO₂, —S(O)R⁷⁰, —S(O)₂R⁷⁰, —S(O)₂O⁻M⁺, —S(O)₂OR⁷⁰, —OS(O)₂R⁷⁰, —OS(O)₂O⁻M⁺, —OS(O)₂OR⁷⁰, —PO₃²⁻(M⁺)₂, —P(O)(OR⁷⁰)O⁻M⁺, —P(O)(OR⁷⁰)(OR⁷⁰), —C(O)R⁷⁰, —C(S)R⁷⁰, —C(NR⁷⁰)R⁷⁰, —C(O)OR⁷⁰, —C(S)OR⁷⁰, —C(O)NR⁸⁰R⁸⁰, —C(NR⁷⁰)NR⁸⁰R⁸⁰, —OC(O)R⁷⁰, —OC(S)R⁷⁰, —OC(O)OR⁷⁰, —OC(S)OR⁷⁰, —NR⁷⁰C(O)R⁷⁰, —NR⁷⁰C(S)R⁷⁰, —NR⁷⁰C(O)OR⁷⁰, —NR⁷⁰C(S)OR⁷⁰, —NR⁷⁰C(O)NR⁸⁰R⁸⁰, —NR⁷⁰C(NR⁷⁰)R⁷⁰ and —NR⁷⁰C(NR⁷⁰)NR⁸⁰R⁸⁰, where R⁶⁰, R⁷⁰, R⁸⁰ and M⁺ are as previously defined.

In addition to the disclosure herein, in a certain embodiment, a group that is substituted has 1, 2, 3, or 4 substituents, 1, 2, or 3 substituents, 1 or 2 substituents, or 1 substituent.

It is understood that in all substituted groups defined above, polymers arrived at by defining substituents with further substituents to themselves (e.g., substituted aryl having a substituted aryl group as a substituent which is itself substituted with a substituted aryl group, which is further substituted by a substituted aryl group, etc.) are not intended for inclusion herein. In such cases, the maximum number of such substitutions is three. For example, serial substitutions of substituted aryl groups specifically contemplated herein are limited to substituted aryl-(substituted aryl)-substituted aryl.

Unless indicated otherwise, the nomenclature of substituents that are not explicitly defined herein are arrived at by naming the terminal portion of the functionality followed by the adjacent functionality toward the point of attachment. For example, the substituent “arylalkyloxycarbonyl” refers to the group (aryl)-(alkyl)-O—C(O)—.

As to any of the groups disclosed herein which contain one or more substituents, it is understood, of course, that such groups do not contain any substitution or substitution patterns which are sterically impractical and/or synthetically non-feasible. In addition, the subject compounds include all stereochemical isomers arising from the substitution of these compounds.

The term “pharmaceutically acceptable salt” means a salt which is acceptable for administration to a patient, such as a mammal (salts with counterions having acceptable mammalian safety for a given dosage regime). Such salts can be derived from pharmaceutically acceptable inorganic or organic bases and from pharmaceutically acceptable inorganic or organic acids. “Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, formate, tartrate, besylate, mesylate, acetate, maleate, oxalate, and the like.

A wavy line in the structure drawing of a group represents an attachment point of the group to the parent structure.

The term “salt thereof” means a compound formed when a proton of an acid is replaced by a cation, such as a metal cation or an organic cation and the like. Where applicable, the salt is a pharmaceutically acceptable salt, although this is not required for salts of intermediate compounds that are not intended for administration to a patient. By way of example, salts of the present compounds include those wherein the compound is protonated by an inorganic or organic acid to form a cation, with the conjugate base of the inorganic or organic acid as the anionic component of the salt.

“Solvate” refers to a complex formed by combination of solvent molecules with molecules or ions of the solute. The solvent can be an organic compound, an inorganic compound, or a mixture of both. Some examples of solvents include, but are not limited to, methanol, N,N-dimethylformamide, tetrahydrofuran, dimethylsulfoxide, and water. When the solvent is water, the solvate formed is a hydrate.

“Stereoisomer” and “stereoisomers” refer to compounds that have same atomic connectivity but different atomic arrangement in space. Stereoisomers include cis-trans isomers, E and Z isomers, enantiomers, and diastereomers.

“Tautomer” refers to alternate forms of a molecule that differ only in electronic bonding of atoms and/or in the position of a proton, such as enol-keto and imine-enamine tautomers, or the tautomeric forms of heteroaryl groups containing a —N═C(H)—NH— ring atom arrangement, such as pyrazoles, imidazoles, benzimidazoles, triazoles, and tetrazoles. A person of ordinary skill in the art would recognize that other tautomeric ring atom arrangements are possible.

It will be appreciated that the term “or a salt or solvate or stereoisomer thereof” is intended to include all permutations of salts, solvates and stereoisomers, such as a solvate of a pharmaceutically acceptable salt of a stereoisomer of subject compound.

As used herein, an “effective dosage” or “effective amount” of drug, compound, conjugate, drug conjugate, antibody drug conjugate, or pharmaceutical composition is an amount sufficient to effect beneficial or desired results. For prophylactic use, beneficial or desired results include results such as eliminating or reducing the risk, lessening the severity, or delaying the onset of the disease, including biochemical, histological and/or behavioral symptoms of the disease, its complications and intermediate pathological phenotypes presenting during development of the disease. For therapeutic use, beneficial or desired results include clinical results such as decreasing one or more symptoms resulting from the disease, increasing the quality of life of those suffering from the disease, decreasing the dose of other medications required to treat the disease, enhancing effect of another medication such as via targeting, delaying the progression of the disease, and/or prolonging survival. In the case of cancer or tumor, an effective amount of the drug may have the effect in reducing the number of cancer cells; reducing the tumor size; inhibiting (i.e., slow to some extent and preferably stop) cancer cell infiltration into peripheral organs; inhibit (i.e., slow to some extent and preferably stop) tumor metastasis; inhibiting, to some extent, tumor growth; and/or relieving to some extent one or more of the symptoms associated with the disorder. An effective dosage can be administered in one or more administrations. For purposes of the present disclosure, an effective dosage of drug, compound, or pharmaceutical composition is an amount sufficient to accomplish prophylactic or therapeutic treatment either directly or indirectly. As is understood in the clinical context, an effective dosage of a drug, compound, or pharmaceutical composition may or may not be achieved in conjunction with another drug, compound, or pharmaceutical composition. Thus, an “effective dosage” may be considered in the context of administering one or more therapeutic agents, and a single agent may be considered to be given in an effective amount if, in conjunction with one or more other agents, a desirable result may be or is achieved.

As used herein, “in conjunction with” refers to administration of one treatment modality in addition to another treatment modality. As such, “in conjunction with” refers to administration of one treatment modality before, during or after administration of the other treatment modality to the individual.

As used herein, “treatment” or “treating” is an approach for obtaining beneficial or desired results including and preferably clinical results. For purposes of the present disclosure, beneficial or desired clinical results include, but are not limited to, one or more of the following: reducing the proliferation of (or destroying) cancerous cells, decreasing symptoms resulting from the disease, increasing the quality of life of those suffering from the disease, decreasing the dose of other medications required to treat the disease, delaying the progression of the disease, and/or prolonging survival of individuals.

As used herein, “delaying development of a disease” means to defer, hinder, slow, retard, stabilize, and/or postpone development of the disease (such as cancer). This delay can be of varying lengths of time, depending on the history of the disease and/or individual being treated. As is evident to one skilled in the art, a sufficient or significant delay can, in effect, encompass prevention, in that the individual does not develop the disease. For example, a late stage cancer, such as development of metastasis, may be delayed.

An “individual” or a “subject” is a mammal, more preferably a human. Mammals also include, but are not limited to, farm animals, sport animals, pets (such as cats, dogs, horses), primates, mice and rats.

As used herein, the term “specifically recognizes” or “specifically binds” refers to measurable and reproducible interactions such as attraction or binding between a target and an antibody (or a molecule or a moiety), that is determinative of the presence of the target in the presence of a heterogeneous population of molecules including biological molecules. For example, an antibody that specifically or preferentially binds to an epitope is an antibody that binds this epitope with greater affinity, avidity, more readily, and/or with greater duration than it binds to other epitopes of the target or non-target epitopes. It is also understood that, for example, an antibody (or moiety or epitope) that specifically or preferentially binds to a first target may or may not specifically or preferentially bind to a second target. As such, “specific binding” or “preferential binding” does not necessarily require (although it can include) exclusive binding. An antibody that specifically binds to a target may have an association constant of at least about 10³ M⁻¹ or 10⁴ M⁻¹, sometimes about 10⁵ M⁻¹ or 10⁶ M⁻¹, in other instances about 10⁶ M⁻¹ or 10⁷ M⁻¹, about 10⁸ M⁻¹ to 10⁹ M⁻¹, or about 10 M⁻¹ to 10¹¹M⁻¹ or higher. A variety of immunoassay formats can be used to select antibodies specifically immunoreactive with a particular protein. For example, solid-phase ELISA immunoassays are routinely used to select monoclonal antibodies specifically immunoreactive with a protein. See, e.g., Harlow and Lane (1988) Antibodies, A Laboratory Manual, Cold Spring Harbor Publications, New York, for a description of immunoassay formats and conditions that can be used to determine specific immunoreactivity.

As used herein, the terms “cancer,” “tumor,” “cancerous,” and “malignant” refer to or describe the physiological condition in mammals that is typically characterized by unregulated cell growth. Examples of cancer include but are not limited to, carcinoma, including adenocarcinoma, lymphoma, blastoma, melanoma, and sarcoma. More particular examples of such cancers include squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, glioma, ovarian cancer, liver cancer such as hepatic carcinoma and hepatoma, bladder cancer, breast cancer, colon cancer, colorectal cancer, endometrial or uterine carcinoma, salivary gland carcinoma, kidney cancer such as renal cell carcinoma and Wilms' tumors, basal cell carcinoma, melanoma, mesothelioma, prostate cancer, thyroid cancer, testicular cancer, esophageal cancer, gallbladder cancer, and various types of head and neck cancer.

As used herein and in the appended claims, the singular forms “a,” “an,” and “the” include plural reference unless the context clearly indicates otherwise. For example, reference to an “antibody” is a reference to from one to many antibodies, such as molar amounts, and includes equivalents thereof known to those skilled in the art, and so forth.

Reference to “about” a value or parameter herein includes (and describes) embodiments that are directed to that value or parameter per se. For example, description referring to “about X” includes description of “X.”

It is understood that aspect and variations of the invention described herein include “consisting” and/or “consisting essentially of” aspects and variations.

Unless defined otherwise, all technical and scientific terms used herein have the same meaning as commonly understood by one of ordinary skill in the art to which this invention belongs. Although any methods and materials similar or equivalent to those described herein can also be used in the practice or testing of the present invention, the preferred methods and materials are now described. All publications mentioned herein are incorporated herein by reference to disclose and describe the methods and/or materials in connection with which the publications are cited.

Except as otherwise noted, the methods and techniques of the present embodiments are generally performed according to conventional methods well known in the art and as described in various general and more specific references that are cited and discussed throughout the present specification. See, e.g., Loudon, Organic Chemistry, 4^{th} edition, New York: Oxford University Press, 2002, pp. 360-361, 1084-1085; Smith and March, March's Advanced Organic Chemistry: Reactions, Mechanisms, and Structure, 5^{th} edition, Wiley-Interscience, 2001.

The nomenclature used herein to name the subject compounds is illustrated in the Examples herein. This nomenclature has generally been derived using the commercially-available AutoNom software (MDL, San Leandro, Calif.).

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination. All combinations of the embodiments pertaining to the chemical groups represented by the variables are specifically embraced by the present invention and are disclosed herein just as if each and every combination was individually and explicitly disclosed, to the extent that such combinations embrace compounds that are stable compounds (i.e., compounds that can be isolated, characterized, and tested for biological activity). In addition, all subcombinations of the chemical groups listed in the embodiments describing such variables are also specifically embraced by the present invention and are disclosed herein just as if each and every such sub-combination of chemical groups was individually and explicitly disclosed herein.

### DETAILED DESCRIPTION

The present disclosure provides compounds (anti-CD22 antibody-drug conjugates) with a hydrophilic self-immolative linker, which may be cleavable under appropriate conditions and incorporates a hydrophilic group to provide better solubility of the conjugate. The hydrophilic self immolative linker may provide increased solubility of drug conjugates for cytotoxic drugs which are often hydrophobic. Other advantages of using a hydrophilic self-immolative linker in a drug conjugate include increased stability of the drug conjugate and decreased aggregation of the drug conjugate.

The present disclosure provides antibody-drug conjugates that may have superior serum stability. For example, in contrast to antibody-drug conjugates wherein a hydroxyl group of a drug is linked to a spacer via a labile carbonate linkage that is susceptible to rapid hydrolysis in aqueous buffer or human serum, the antibody-drug conjugates of the present embodiments utilizing a benzyloxycarbonyl linkage may be relatively more stable under the same conditions, and may selectively undergo fragmentation to release the drug upon treatment with protease, e.g., cathepsin B. Serum stability is a desirable property for antibody-drug conjugates where it is desired to administer inactive drug to the patient's serum, have that inactive drug concentrate at a target by way of the ligand, and then have that antibody-drug conjugate converted to an active form only in the vicinity of the target.

The present disclosure provides antibody-drug conjugates which may have decreased aggregation. Increased associated hydrophobicity of some enzyme-labile linkers may lead to aggregation of antibody-drug conjugates, particularly with strongly hydrophobic drugs. With incorporation of a hydrophilic group into the linker, there may be decreased aggregation of the antibody-drug conjugate.

The compounds (antibody-drug conjugates) of the present disclosure comprise a drug moiety, a targeting moiety capable of targeting a selected cell population (e.g., CD22 expressing cells), and a linker which contains an acyl unit, an optional spacer unit for providing distance between the drug moiety and the targeting moiety, a peptide linker which can be cleavable under appropriate conditions, a hydrophilic self-immolative linker, and an optional second self-immolative spacer or cyclization self-elimination linker. Each of the features is discussed below.

The present disclosure provides a compound of Formula (I):

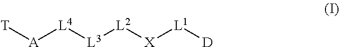

or a salt or solvate or stereoisomer thereof;
wherein:
D is a drug moiety;
T is a targeting moiety;
X is a hydrophilic self-immolative linker;
L¹ is a bond, a second self-immolative linker, or a cyclization self-elimination linker;
L² is a bond or a second self-immolative linker;
   wherein if L¹ is a second self-immolative linker or a cyclization self-elimination linker, then L² is a bond;
   wherein if L² is a second self-immolative linker, then L¹ is a bond;
L³ is a peptide linker;
L⁴ is a bond or a spacer; and
A is an acyl unit.

In some embodiments, the targeting moiety is an antibody that specifically binds to a CD22 (e.g., a human CD22). In some embodiments, the targeting moiety is an anti-CD22 antibody which has one or more attachment sites for linking to the drug moiety. For example, a targeting moiety T can have multiple sites for linking to a linker-drug moiety (e.g., A-L⁴-L³-L²-X-L¹-D). Thus, also provided is a compound of Formula (Ia):

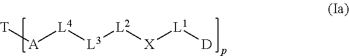

or a salt or solvate or stereoisomer thereof; wherein D, T, X, L¹, L², L³, L⁴ and A are as defined for Formula (I), and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3 or 4. In some embodiments, p is 2. In some embodiments, p is 3. In some embodiments, p is 4.

### Peptide Linker

In Formula (I), L³ is a peptide linker. In certain embodiments, L³ is a peptide linker of 1 to 10 amino acid residues. In certain embodiments, L³ is a peptide linker of 2 to 4 amino acid residues. In certain instances, L³ is a dipeptide linker.

An amino acid residue can be a naturally-occurring or non-natural amino acid residue. The terms “natural amino acid” and “naturally-occurring amino acid” refer to Ala, Asp, Cys, Glu, Phe, Gly, His, Ile, Lys, Leu, Met, Asn, Pro, Gln, Arg, Ser, Thr, Val, Trp, and Tyr. “Non-natural amino acids” (i.e., amino acids do not occur naturally) include, by way of non-limiting example, homoserine, homoarginine, citrulline, phenylglycine, taurine, iodotyrosine, seleno-cysteine, norleucine (“Nle”), norvaline (“Nva”), beta-alanine, L- or D-naphthalanine, ornithine (“Orn”), and the like.

Amino acids also include the D-forms of natural and non-natural amino acids. “D-” designates an amino acid having the “D” (dextrorotary) configuration, as opposed to the configuration in the naturally occurring (“L-”) amino acids. Where no specific configuration is indicated, one skilled in the art would understand the amino acid to be an L-amino acid. The amino acids can, however, also be in racemic mixtures of the D- and L-configuration. Natural and non-natural amino acids can be purchased commercially (Sigma Chemical Co.; Advanced Chemtech) or synthesized using methods known in the art Amino acid substitutions may be made on the basis of similarity in polarity, charge, solubility, hydrophobicity, hydrophilicity, and/or the amphipathic nature of the residues as long as their biological activity is retained.

The amino acid residue sequence can be specifically tailored so that it will be selectively enzymatically cleaved from the resulting peptidyl derivative drug-conjugate by one or more of the tumor-associated proteases.

In certain embodiments, L³ is a peptide linker comprising at least one lysine or arginine residue.

In certain embodiments, L³ is a peptide linker comprising an amino acid residue selected from lysine, D-lysine, citrulline, arginine, proline, histidine, ornithine and glutamine.

In certain embodiments, L³ is a peptide linker comprising an amino acid residue selected from valine, isoleucine, phenylalanine, methionine, asparagine, proline, alanine, leucine, tryptophan, and tyrosine.

In certain embodiments, L³ is a dipeptide linker selected from valine-citrulline, proline-lysine, methionine-D-lysine, asparagine-D-lysine, isoleucine-proline, phenylalanine-lysine, and valine-lysine. In certain embodiments, L³ is valine-citrulline.

Numerous specific peptide linker molecules suitable for use in the present disclosure can be designed and optimized in their selectivity for enzymatic cleavage by a particular tumor-associated protease. Certain peptide linkers for use in the present disclosure are those which are optimized toward the proteases, cathepsin B and D.

### Hydrophilic Self-Immolative Linker

In Formula (I), X is a hydrophilic self-immolative linker.

The compound of the present disclosure employs a hydrophilic self-immolative spacer moiety which spaces and covalently links together the drug moiety and the targeting moiety and incorporates a hydrophilic group, which provides better solubility of the compound. Increased associated hydrophobicity of some enzyme-labile linkers can lead to aggregation of drug conjugates, particularly with strongly hydrophobic drugs. With incorporation of a hydrophilic group into the linker, there may be a decreased aggregation of the drug conjugate.

A self-immolative spacer may be defined as a bifunctional chemical moiety which is capable of covalently linking together two spaced chemical moieties into a normally stable tripartite molecule, can release one of the spaced chemical moieties from the tripartite molecule by means of enzymatic cleavage; and following enzymatic cleavage, can spontaneously cleave from the remainder of the molecule to release the other of the spaced chemical moieties.

In certain embodiments, X is a benzyloxycarbonyl group. In certain embodiments, X is

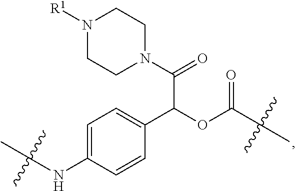

wherein R¹ is hydrogen, unsubstituted or substituted C₁₋₃ alkyl, or unsubstituted or substituted heterocyclyl.

In such instance, the present disclosure provides a compound of Formula (II):

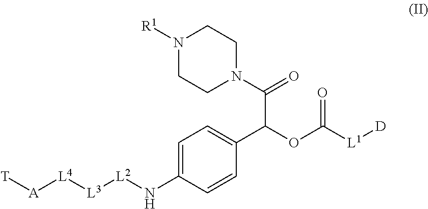

or a salt or solvate or stereoisomer thereof;

wherein:

D is a drug moiety;

T is a targeting moiety;

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond, a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit.

In some embodiments, provided is a compound of Formula (IIa):

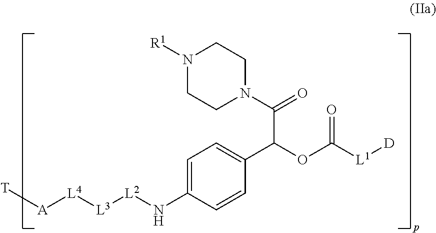

or a salt or solvate or stereoisomer thereof; wherein D, T, L¹, L², L³, L⁴ and A are as defined for Formula (II), and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3 or 4. In some embodiments, p is 2. In some embodiments, p is 3. In some embodiments, p is 4.

In certain embodiments of Formula (II) or (IIa), R¹ is hydrogen. In certain instances, R¹ is methyl.

It is intended and understood that each and every variation of D, T, L¹, L², L³, L⁴ and A described for formula (I) or (Ia) may be applied to Formula (II) or (IIa) as if each and every variation and combinations thereof is individually described. For example, in some embodiments, the targeting moiety of formula (II) or (IIa) is an antibody that specifically binds to a CD22 (e.g., a human CD22). It is further intended and understood that each and every variation of one of D, T, L¹, L², L³, L⁴ and A described for formula (I) may be combined with each and every variation of another one of D, T, L¹, L², L³, L⁴ and A described for formula (I), where applicable, as if each and every combination is individually described.

The release of the drug moiety is based on the self-elimination reaction of aminobenzyloxycarbonyl group. For illustration purposes, a reaction scheme with an aminobenzyloxycarbonyl group with a drug and peptide attached is shown below.

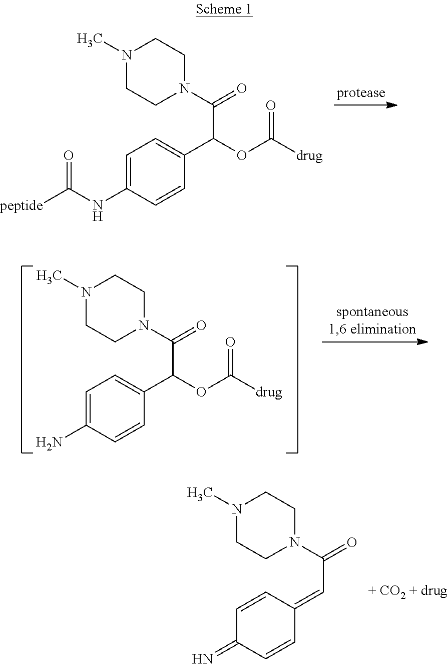

Referring to Scheme 1, upon cleavage from a peptide, an aminobenzyloxycarbonyl is formed and is able to undergo a spontaneous 1,6 elimination to form a cyclohexa-2,5-dienimine derivative and carbon dioxide and release the drug.

### Optional Second Self-Immolative Linker or Cyclization Self-elimination Linker

A second self-immolative linker or cyclization self-elimination linker provides an additional linker for allowance of fine-tuning the cleavage of the compound to release the drug moiety.

In Formula (I) or (Ia), L¹ is a bond, a second self-immolative linker, or a cyclization self-elimination linker; L² is a bond or a second self-immolative linker; wherein if L¹ is a second self-immolative linker or a cyclization self-elimination linker, then L² is a bond; and wherein if L² is a second self-immolative linker, then L¹ is a bond. Thus, there is an optional second self-immolative linker or a cyclization self-elimination linker adjacent the hydrophilic self-immolative linker.

In certain embodiments, L¹ is a bond and L² is a bond. In certain embodiments, L¹ is a second self-immolative linker or a cyclization self-elimination linker and L² is a bond. In certain embodiments, L¹ is a bond and L² is a second self-immolative linker.

In Formula (I) or (Ia), in certain embodiments, L¹ is a bond. In certain embodiments, L¹ is a second self-immolative spacer or a cyclization self-elimination linker, which separates the hydrophilic self-immolative linker and the drug moiety. In certain embodiments, L¹ is an aminobenzyloxycarbonyl linker.

In certain embodiments, L¹ is selected from:

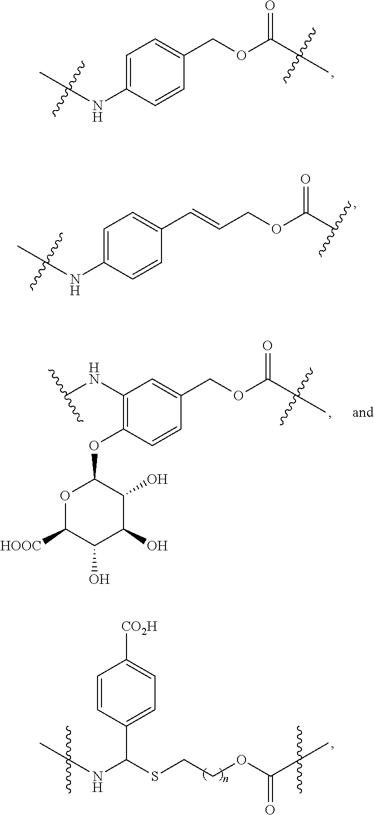

wherein n is 1 or 2.

In certain instances, the second self-immolative linker or cyclization self-elimination linker provides design potential for a wider variety of moieties that can be used. For example, in Formula (II) or (IIa), a carbamate linkage (—O—C(O)—N(H)—) linkage between the hydrophilic self-immolative linker and the drug moiety would provide a stable drug conjugate and would readily cleave to provide a free drug moiety. The hydrophilic self-immolative linker will typically terminate with an oxycarbonyl group (—O—C(O)—). If the drug moiety has an amino-reactive group that may be used to react to form a carbamate group, then the second self-immolative unit or cyclization self-elimination linker is not necessary; although it may still be employed. However, if the drug does not contain an amino group, but instead contains some other reactive functional group, then such drugs may still be incorporated into an aminobenzyloxycarbonyl-containing compound of the present embodiments by including a second, intermediate self-immolative spacer or cyclization self-elimination linker between the drug moiety and the aminobenzyloxycarbonyl group.

The cyclization self-elimination linkers of L¹ below provide linkage of hydroxyl-containing or thiol-containing drug moieties to the aminobenzyloxycarbonyl group of the hydrophilic self-immolative linker:

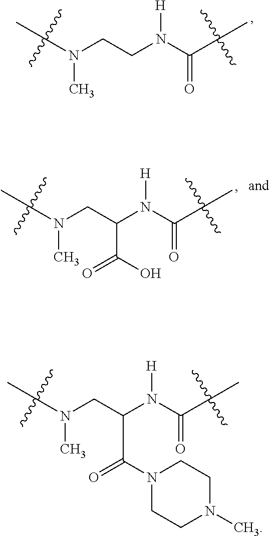

The cyclization self-elimination linkers in the compounds of the embodiments provide for cleavage of the compound to release the drug moiety. The elimination mechanism of the adjacent hydrophilic self-immolative linker would reveal an amino group of L¹. The amino group can then react with the carbamate group or thiocarbamate linkage of L¹ and the drug moiety in a cyclization reaction to release the hydroxyl-containing or thiol-containing drug moiety.

In Formula (I) or (Ia), in certain embodiments, L² is a bond. In certain embodiments, L² is a second self-immolative spacer which separates the hydrophilic self-immolative linker and the peptide linker. In certain embodiments, L² is an aminobenzyloxycarbonyl linker.

In certain embodiments, L² is selected from

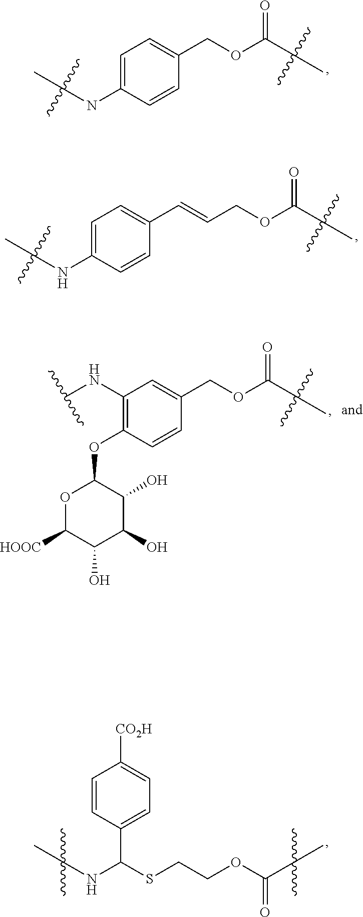

wherein n is 1 or 2.

### Optional Spacer

In Formula (I) or (Ia), L⁴ is a bond or a spacer. In certain embodiments, L⁴ is a bond. In certain embodiments, L⁴ is a spacer, which can provide distance between the drug moiety and the targeting moiety.

In certain embodiments, a spacer is selected from alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic, and heteroatoms, and combinations thereof. The spacer can be homogenous or heterogeneous in its atom content (e.g., spacers containing only carbon atoms or spacers containing carbon atoms as well as one or more heteroatoms present on the spacer. Preferably, the spacer contains 1 to 50 carbon atoms and 0 to 30 heteroatoms selected from oxygen, nitrogen and sulfur. The spacer may also be chiral or achiral, linear, branched or cyclic.

In certain embodiments, L⁴ is a spacer selected from polyalkylene glycol, alkylene, alkenylene, alkynylene, and polyamine. Examples of alkenylene include, but is not limited to, vinylene (—CH═CH—), allylene (—CH₂C═C—), and but-3-en-1-ylene (—CH₂ CH₂C═CH—). Examples of alkynylene include, but are not limited to, acetylenylene (—C≡C—), and propargylene (—CH₂C≡C—).

In certain embodiments, L⁴ is a spacer that comprises a functional group that can provide linkage to the terminal end of the peptide linkage. Functional groups, such as C(O), C(O)—NH, S(O)₂, and S(O)₂—NH, can provide linkage to the terminal end of the peptide linkage. In certain instances, L⁴ is L^{4a}-C(O), L^{4a}-C(O)—NH, L^{4a}-S(O)₂, L^{4a}-S(O)₂—NH, wherein L^{4a} is selected from polyalkylene glycol, alkylene, alkenylene, alkynylene, and polyamine. In certain instances, L⁴ is L^{4a}-C(O), wherein L^{4a} is selected from polyalkylene glycol, alkylene, alkenylene, alkynylene, and polyamine.

In certain embodiments, L⁴ is L^{4a}-C(O), wherein L^{4a} is a polyalkylene glycol. In certain embodiments, L⁴ is L^{4a}-C(O), wherein L^{4a} is a polyethylene glycol. In certain embodiments, the spacer is of the formula —CH₂—(CH₂—O—CH₂)ₘ—CH₂—C(O)—, wherein m is an integer from 0 to 30.

In certain embodiments, L⁴ is L^{4a}-C(O), wherein L^{4a} is alkylene. In certain embodiments, L⁴ is L^{4a}-C(O), wherein L^{4a} is C₁₋₁₀alkylene, C₁₋₈alkylene, or C₁₋₆alkylene. In certain embodiments, L⁴ is L^{4a}-C(O), wherein L^{4a} is C₄alkylene, C₅alkylene, or C₆alkylene. In certain embodiments, L⁴ is L^{4a}-C(O), wherein L^{4a} is C₅alkylene.

### Acyl Unit

In Formula (I) or (Ia), A is an acyl unit. In certain embodiments, the acyl unit “A” comprises a sulfur atom and is linked to the targeting moiety via a sulfur atom derived from the targeting moiety. In such instance, a dithio bond is formed between the acyl unit and the targeting moiety.

In certain embodiments, A is selected from

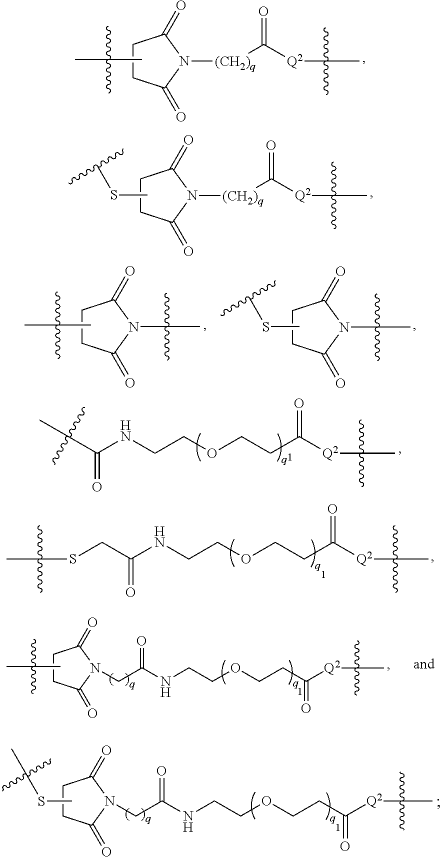

wherein Q² is NH or O, each q is independently an integer from 1 to 10, and each q₁ is independently an integer from 1 to 10. In some embodiments, q is an integer from 2 to 5, such as 2, 3, 4, or 5. In some embodiments, q₁ is an integer from 2 to 5, such as 2, 3, 4, or 5.

In certain embodiments, A is

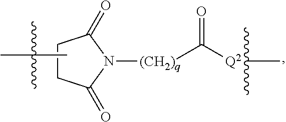

wherein Q² is NH or O and q is an integer from 1 to 10. In certain instance, q is a number from 2 to 5, such as 2, 3, 4, or 5.

In certain embodiments, A is

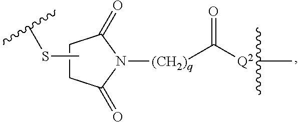

wherein Q² is NH or O and q is an integer from 1 to 10. In certain instance, q is a number from 2 to 5, such as 2, 3, 4, or 5.

In certain embodiments, A is selected from

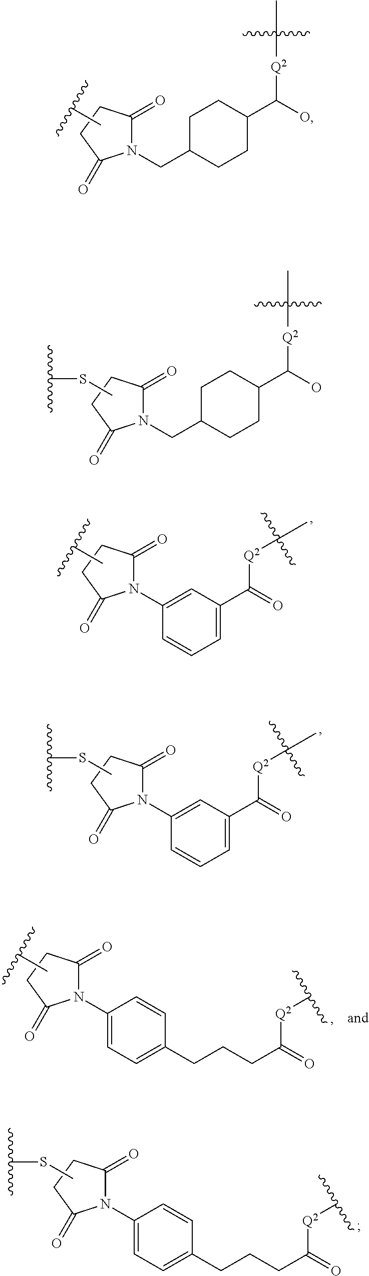

wherein Q² is NH or 0.

### Drug Moiety

The drug conjugates of the present embodiments are effective for the usual purposes for which the corresponding drugs are effective, and have superior efficacy because of the ability, inherent in the targeting moiety, to transport the drug to the desired cell where it is of particular benefit.

The preferred drugs for use in the present embodiments are cytotoxic drugs, such as those which are used for cancer therapy. Such drugs include, in general, DNA damaging agents, anti-metabolites, natural products and their analogs. Certain classes of cytotoxic agents include, for example, the enzyme inhibitors such as dihydrofolate reductase inhibitors, thymidylate synthase inhibitors, DNA intercalators, DNA cleavers, topoisomerase inhibitors, the anthracycline family of drugs, the vinca drugs, the mitomycins, the bleomycins, the cytotoxic nucleosides, the pteridine family of drugs, diynenes, the podophyllotoxins, differentiation inducers, and taxols. Certain useful members of those classes include, for example, methotrexate, methopterin, dichloromethotrexate, 5-fluorouracil, 6-mercaptopurine, cytosine arabinoside, melphalan, leurosine, leurosideine, actinomycin, daunorubicin, doxorubicin, mitomycin C, mitomycin A, carminomycin, aminopterin, tallysomycin, podophyllotoxin and podophyllotoxin derivatives such as etoposide or etoposide phosphate, vinblastine, vincristine, vindesine, taxol, taxotere retinoic acid, butyric acid, N⁸-acetyl spermidine, camptothecin, and their analogues. Other drugs include dolastatin and duocarmycin.

One skilled in the art may make chemical modifications to the desired compound in order to make reactions of that compound more convenient for purposes of preparing conjugates of the invention.

In certain embodiments, D is a drug moiety having a chemically reactive functional group by means of which the drug is bonded to L¹ or X. In certain instances, the functional group is selected from a primary amine, a secondary amine, hydroxyl, and sulfhydryl. In certain instances, the functional group is a primary amine or a secondary amine. In certain instances, the functional group is hydroxyl. In certain instances, the functional group is sulfhydryl.

As discussed above, the hydrophilic self-immolative linker will typically terminate with an oxycarbonyl group (—O—C(O)—). Thus, an amino-containing drug moiety would readily react with the oxycarbonyl group to form a carbamate group. In certain embodiments, D is an amino-containing drug moiety, wherein the drug is connected to L¹ or X through the amino group.

However, if the drug moiety does not contain an amino group, the second self-immolative linker or cyclization self-elimination linker of L¹ can provide design potential for a wider variety of moieties that can be used. In certain embodiments, D is a hydroxyl-containing or sulfhydryl-containing drug moiety, wherein the drug is connected to L¹ through the hydroxyl or sulfhydryl group.

Representative amino-containing drugs include mitomycin-C, mitomycin-A, daunorubicin, doxorubicin, aminopterin, actinomycin, bleomycin, 9-amino camptothecin, N⁸-acetyl spermidine, 1-(2-chloroethyl)-1,2-dimethanesulfonyl hydrazide, tallysomycin, cytarabine, dolastatin and derivatives thereof. Amino-containing drugs also include amino derivatives of drugs that do not naturally contain an amino group. In certain embodiments, D is duocarmycin, dolastatin, tubulysin, doxorubicin (DOX), paclitaxel, or mitomycin C (MMC), or amino derivatives thereof.

Representative hydroxyl-containing drugs include etoposide, camptothecin, taxol, esperamicin, 1,8-dihydroxy-bicyclo[7.3.1]trideca-4-9-diene-2,6-diyne-13-one, (U.S. Pat. No. 5,198,560), podophyllotoxin, anguidine, vincristine, vinblastine, morpholine-doxorubicin, n-(5,5-diacetoxy-pentyl) doxorubicin, duocarmycin, and derivatives thereof.

Representative sulfhydryl-containing drugs include esperamicin and 6-mercaptopurine, and derivatives thereof.

A certain group of cytotoxic agents for use as drugs in the present embodiments include drugs of the following formulae:

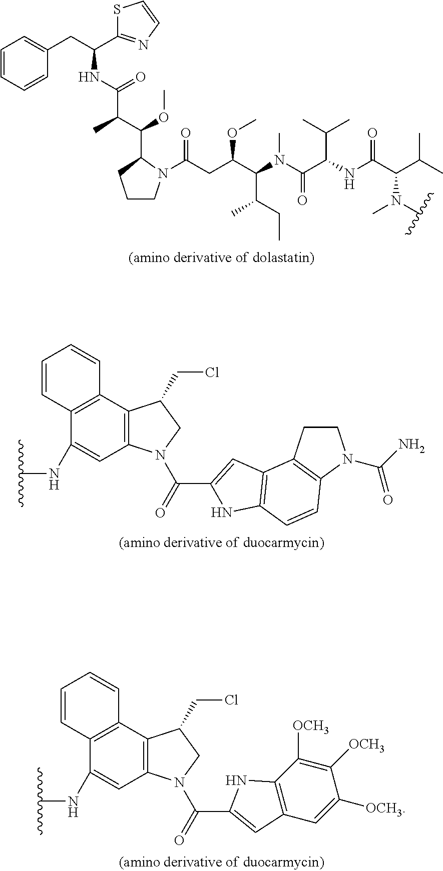 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C)C(C)C ; *Nc1cc2c(c3ccccc13)[C@H](CCl)CN2C(=O)c1cc2c3c(ccc2[nH]1)N(C(N)=O)CC3 ; *Nc1cc2c(c3ccccc13)[C@H](CCl)CN2C(=O)c1cc2cc(OC)c(OC)c(OC)c2[nH]1]

### Targeting Moiety

A targeting moiety as described in the present disclosure refers to a moiety or molecule that specifically binds, complexes with, reacts with, or associates with a given cell population (e.g., a CD22 expressing cells). In a conjugate described herein, a targeting moiety described herein is linked via a linker to a drug moiety in the conjugate. In some embodiments, the targeting moiety is capable of delivering a drug moiety (e.g., a drug moiety used for therapeutic purpose) to a particular target cell population which the targeting moiety binds, complexes with, reacts with, or associates with.

In some embodiments, the targeting moiety is an antibody (or an antibody moiety or an antibody targeting moiety). In some embodiments, the targeting moiety comprises sulfhydryl (—SH) group (e.g., a free reactive sulfhydryl (—SH) group) or can be modified to contain such a sulfhydryl group. In some embodiments, the targeting moiety comprises an antibody with a sulfhydryl group (e.g., a free reactive sulfhydryl group). In some embodiments, the targeting moiety comprises a free thiol group such as an antibody with a free thiol group or can be modified to contain such a thio group. In some embodiments, the targeting moiety comprising a sulfhydryl group or thiol group bonds to a linker via the sulfur atom in the sulfhydryl group.

In some embodiments, the targeting moiety (e.g., an antibody targeting moiety) has one or more attachment sites for linking to the drug moiety. For example, a targeting moiety T (e.g., an antibody) can have multiple sites (e.g., multiple sulfhydryl groups) for linking to a linker-drug moiety (e.g., A-L⁴-L³-L²-X-L¹-D where A is suitable for bonding to a sulfhydryl group of the targeting antibody). In some embodiments, the targeting moiety can have 1 to 20 sites of attachment. In some embodiments, the targeting moiety can have 1 to 20, 1 to 10, 1 to 8, 1 to 6, 1 to 4, 2 to 8, 2 to 6, or 2 to 4 sites of attachment. In some embodiments, the targeting moiety has 1, 2, 3, 4, 5, 6, 7, or 8 sites of attachment. In some embodiments, the targeting moiety has 2 sites of attachment. In some embodiments, the targeting moiety has 1 site of attachment. In some embodiments, the targeting moiety has 4 sites of attachment. In some instances, certain potential sites of attachment may not be accessible for bonding to a drug moiety. Thus, the number of attachment sites in a targeting moiety T may result in a drug conjugate that has fewer number of drug moieties attached than the number of potential sites of attachment. In some embodiments, one or more of the sites of attachment may be accessible for bonding a drug moiety. For example, an antibody targeting moiety can have one or two sulfhydryl groups on each chain of the antibody accessible for bonding to drug moiety via a linker.

An antibody described herein refers to an immunoglobulin molecule capable of specific binding to a target (i.e., CD22) through at least one antigen recognition site, located in the variable region of the immunoglobulin molecule. As used herein, the term “antibody” encompasses not only intact polyclonal or monoclonal antibodies, but also antigen-binding fragments thereof (such as Fab, Fab′, F(ab′)₂, Fv), single chain (ScFv), mutants thereof, fusion proteins comprising an antibody portion, and any other modified configuration of the immunoglobulin molecule that comprises an antigen recognition site. An antibody includes an antibody of any class, such as IgG, IgA, or IgM (or sub-class thereof), and the antibody need not be of any particular class. Depending on the antibody amino acid sequence of the constant domain of its heavy chains, immunoglobulins can be assigned to different classes. There are five major classes of immunoglobulins: IgA, IgD, IgE, IgG, and IgM, and several of these may be further divided into subclasses (isotypes), e.g., IgG1, IgG2, IgG3, IgG4, IgA1 and IgA2. The heavy-chain constant domains that correspond to the different classes of immunoglobulins are called alpha, delta, epsilon, gamma, and mu, respectively. The subunit structures and three-dimensional configurations of different classes of immunoglobulins are well known.

An antibody included or used in a targeting moiety described herein (or an antibody targeting moiety) can encompass monoclonal antibodies, polyclonal antibodies, antibody fragments (e.g., Fab, Fab′, F(ab′)₂, Fv, Fc, etc.), chimeric antibodies, humanized antibodies, human antibodies (e.g., fully human antibodies), single chain (ScFv), bispecific antibodies, multispecific antibodies, mutants thereof, fusion proteins comprising an antibody portion, and any other modified configuration of the immunoglobulin molecule that comprises an antigen recognition site of the required specificity. The antibodies may be murine, rat, camel, human, or any other origin (including humanized antibodies). In some embodiments, an antibody used in a targeting moiety described herein (or an antibody targeting moiety) is any one of the following: bispecific antibody, multispecific, single-chain, bifunctional, and chimeric and humanized molecules having affinity for a polypeptide conferred by at least one hypervariable region (HVR) or complementarity determining region (CDR) of the antibody. Antibodies used in the present disclosure also include single domain antibodies which are either the variable domain of an antibody heavy chain or the variable domain of an antibody light chain. Holt et al., Trends Biotechnol. 21:484-490, 2003. Methods of making domain antibodies comprising either the variable domain of an antibody heavy chain or the variable domain of an antibody light chain, containing three of the six naturally occurring HVRs or CDRs from an antibody, are also known in the art. See, e.g., Muyldermans, Rev. Mol. Biotechnol. 74:277-302, 2001.

In some embodiments, an antibody included or used in a targeting moiety described herein (or an antibody targeting moiety) is a monoclonal antibody. As used herein, a monoclonal antibody refers to an antibody of substantially homogeneous antibodies, i.e., the individual antibodies comprising the population are identical except for possible naturally-occurring mutations that may be present in minor amounts. Furthermore, in contrast to polyclonal antibody preparations, which typically include different antibodies directed against different determinants (epitopes), monoclonal antibody is not a mixture of discrete antibodies. The modifier “monoclonal” indicates the character of the antibody as being obtained from a substantially homogeneous population of antibodies, and is not to be construed as requiring production of the antibody by any particular method. For example, the monoclonal antibodies used in the present disclosure may be made by the hybridoma method first described by Kohler and Milstein, 1975, Nature, 256:495, or may be made by recombinant DNA methods such as described in U.S. Pat. No. 4,816,567. The monoclonal antibodies may also be isolated from phage libraries generated using the techniques described in McCafferty et al., 1990, Nature, 348:552-554, for example.

In some embodiments, an antibody included or used in a targeting moiety described herein (or an antibody targeting moiety) is a chimeric antibody. As used herein, a chimeric antibody refers to an antibody having a variable region or part of variable region from a first species and a constant region from a second species. An intact chimeric antibody comprises two copies of a chimeric light chain and two copies of a chimeric heavy chain. The production of chimeric antibodies is known in the art (Cabilly et al. (1984), Proc. Natl. Acad. Sci. USA, 81:3273-3277; Harlow and Lane (1988), Antibodies: a Laboratory Manual, Cold Spring Harbor Laboratory). Typically, in these chimeric antibodies, the variable region of both light and heavy chains mimics the variable regions of antibodies derived from one species of mammals, while the constant portions are homologous to the sequences in antibodies derived from another. One clear advantage to such chimeric forms is that, for example, the variable regions can conveniently be derived from presently known sources using readily available hybridomas or B cells from non-human host organisms in combination with constant regions derived from, for example, human cell preparations. While the variable region has the advantage of ease of preparation, and the specificity is not affected by its source, the constant region being human is less likely to elicit an immune response from a human subject when the antibodies are injected than would the constant region from a non-human source. However, the definition is not limited to this particular example.

In some embodiments, an antibody included or used in a targeting moiety described herein (or an antibody targeting moiety) is a humanized antibody. As used herein, humanized antibodies refer to forms of non-human (e.g. murine) antibodies that are specific chimeric immunoglobulins, immunoglobulin chains, or fragments thereof (such as Fv, Fab, Fab′, F(ab′)₂ or other antigen-binding subsequences of antibodies) that contain minimal sequence derived from non-human immunoglobulin. For the most part, humanized antibodies are human immunoglobulins (recipient antibody) in which residues from a HVR or CDR of the recipient are replaced by residues from a HVR or CDR of a non-human species (donor antibody) such as mouse, rat, or rabbit having the desired specificity, affinity, and capacity. In some instances, Fv framework region (FR) residues of the human immunoglobulin are replaced by corresponding non-human residues. Furthermore, the humanized antibody may comprise residues that are found neither in the recipient antibody nor in the imported HVR or CDR or framework sequences, but are included to further refine and optimize antibody performance. In general, the humanized antibody will comprise substantially all of at least one, and typically two, variable domains, in which all or substantially all of the HVR or CDR regions correspond to those of a non-human immunoglobulin and all or substantially all of the FR regions are those of a human immunoglobulin consensus sequence. The humanized antibody optimally also will comprise at least a portion of an immunoglobulin constant region or domain (Fc), typically that of a human immunoglobulin. Antibodies may have Fc regions modified as described in WO 99/58572. Other forms of humanized antibodies have one or more HVRs or CDRs (one, two, three, four, five, six) which are altered with respect to the original antibody, which are also termed one or more HVRs or CDRs “derived from” one or more HVRs or CDRs from the original antibody.

In some embodiments, an antibody included or used in a targeting moiety described herein (or an antibody targeting moiety) is a human antibody. As used herein, a human antibody means an antibody having an amino acid sequence corresponding to that of an antibody produced by a human and/or has been made using any of the techniques for making human antibodies known in the art. A human antibody used herein includes antibodies comprising at least one human heavy chain polypeptide or at least one human light chain polypeptide. One such example is an antibody comprising murine light chain and human heavy chain polypeptides. Human antibodies can be produced using various techniques known in the art. In one embodiment, the human antibody is selected from a phage library, where that phage library expresses human antibodies (Vaughan et al., 1996, Nature Biotechnology, 14:309-314; Sheets et al., 1998, PNAS, (USA) 95:6157-6162; Hoogenboom and Winter, 1991, J. Mol. Biol., 227:381; Marks et al., 1991, J. Mol. Biol., 222:581). Human antibodies can also be made by introducing human immunoglobulin loci into transgenic animals, e.g., mice in which the endogenous immunoglobulin genes have been partially or completely inactivated. This approach is described in U.S. Pat. Nos. 5,545,807; 5,545,806; 5,569,825; 5,625,126; 5,633,425; and 5,661,016. Alternatively, the human antibody may be prepared by immortalizing human B lymphocytes that produce an antibody directed against a target antigen (such B lymphocytes may be recovered from an individual or may have been immunized in vitro). See, e.g., Cole et al., Monoclonal Antibodies and Cancer Therapy, Alan R. Liss, p. 77 (1985); Boerner et al., 1991, J. Immunol., 147 (1):86-95; and U.S. Pat. No. 5,750,373.

In some embodiments, an antibody included or used in a targeting moiety described herein (or an antibody targeting moiety) specifically binds to a CD22 (e.g., a human CD22). In some embodiments, an antibody included or used in a targeting moiety described herein (or an antibody targeting moiety) specifically binds to an extracellular domain of CD22 (e.g., an extracellular domain of a human CD22). As used herein, “CD22” refers to both wild type sequences and naturally occurring variant sequences. A non-limiting example of a CD22 recognized by antibodies of this invention is human CD22 (Accession No. Protein Data Base: NP_{—}001762.2; GenBank No.: NM_{—}001771.3), the amino acid sequence of which is provided below:

(SEQ ID NO: 14)
MHLLGPWLLLLVLEYLAFSDSSKWVFEHPETLYAWEGACVWIPCTYRALD
GDLESFILFHNPEYNKNTSKFDGTRLYESTKDGKVPSEQKRVQFLGDKNK
NCTLSIHPVHLNDSGQLGLRMESKTEKWMERIHLNVSERPFPPHIQLPPE
IQESQEVTLTCLLNFSCYGYPIQLQWLLEGVPMRQAAVTSTSLTIKSVFI
RSELKESPQWSHHGKIVTCQLQDADGKELSNDTVQLNVKHTPKLEIKVTP
SDAIVREGDSVTMTCEVSSSNPEYTTVSWLKDGTSLKKQNTFTLNLREVT
KDQSGKYCCQVSNDVGPGRSEEVFLQVQYAPEPSTVQILHSPAVEGSQVE
FLCMSLANPLPTNYTWYHNGKEMQGRTEEKVHIPKILPWHAGTYSCVAEN
ILGTGQRGPGAELDVQYPPKKVTTVIQNPMPIREGDTVTLSCNYNSSNPS
VTRYEWKPHGAWEEPSLGVLKIQNVGWDNTTIACAACNSWCSWASPVALN
VQYAPRDVRVRKIKPLSEIHSGNSVSLQCDFSSSHPKEVQFFWEKNGRLL
GKESQLNFDSISPEDAGSYSCWVNNSIGQTASKAWTLEVLYAPRRLRVSM
SPGDQVMEGKSATLTCESDANPPVSHYTWEDWNNQSLPYHSQKLRLEPVK
VQHSGAYWCQGTNSVGKGRSPLSTLTVYYSPETIGRRVAVGLGSCLAILI
LAICGLKLQRRWKRTQSQQGLQENSSGQSFFVRNKKVRRAPLSEGPHSLG
CYNPMMEDGISYTTLRFPEMNIPRTGDAESSEMQRPPPDCDDTVTYSALH
KRQVGDYENVIPDFPEDEGIHYSELIQFGVGERPQAQENVDYVILKH.

In some embodiments, the anti-CD22 antibody described herein binds to a mature CD22 (e.g., a human CD22) expressed on the cell surface of a human cell (e.g., a human cancer cell). In some embodiments, the anti-CD22 antibody described herein binds a mature CD22 expressed on the cell surface of a human lymphoma cell or a human leukemia cell.

Examples of the anti-CD22 antibodies and their amino acid sequences are provided below in Table 1.

TABLE 1
Amino Acid Sequences of anti-CD22 antibodies

| SEQ | |
|---|---|
| ID | |
| NO. | DESCRIPTION |
| 1 | Amino acid sequence of hLL2 light chain variable region |
| 2 | Amino acid sequence of hLL2 heavy chain variable region |
| 3 | Amino acid sequence of h10F4 light chain variable region |
| 4 | Amino acid sequence of h10F4 heavy chain variable region |
| 5 | Amino acid sequence of g5/44 light chain variable region |
| 6 | Amino acid sequence of g5/44 heavy chain variable region |
| 7 | Amino acid sequence of hHB22.7 light chain variable region |
| 8 | Amino acid sequence of hHB22.7 heavy chain variable region |
| 9 | Amino acid sequence of RFB4 light chain variable region |
| 10 | Amino acid sequence of RFB4 heavy chain variable region |
| 11 | Amino acid sequence of human kappa light chain constant domain |
| 12 | Amino acid sequence of human IgG1 heavy chain constant domain |
| 13 | Amino acid sequence of human IgG4p |
| | heavy chain constant domain |
| 45 | Amino acid sequence of hLL2 light chain |
| 46 | Amino acid sequence of hLL2 heavy chain comprising human |
| | IgG1 constant domain |
| 47 | Amino acid sequence of hLL2 heavy chain comprising human |
| | IgG4p constant domain |
| 48 | Amino acid sequence of h10F4 light chain |
| 49 | Amino acid sequence of h10F4 heavy chain comprising human |
| | IgG1 constant domain |
| 50 | Amino acid sequence of h10F4 heavy chain comprising human |
| | IgG4p constant domain |
| 51 | Amino acid sequence of g5/44 light chain region |
| 52 | Amino acid sequence of g5/44 heavy chain comprising human |
| | IgG1 constant domain |
| 53 | Amino acid sequence of g5/44 heavy chain comprising human |
| | IgG4p constant domain |
| 54 | Amino acid sequence of hHB22.7 light chain |
| 55 | Amino acid sequence of hHB22.7 heavy chain comprising human |
| | IgG1 constant domain |
| 56 | Amino acid sequence of hHB22.7 heavy chain comprising human |
| | IgG4p constant domain |

IgG4p denotes the human IgG4 antibody with mutation of Ser228 to Pro (S228P), which would prevent Fab arm exchange with another IgG4 in vivo (Stubenrauch et al., (2010) Drug Metab Dispos. 38(1):84-91).

The amino acid sequences of the heavy chain variable regions and light chain variable regions of hLL2, h10F4, g5/44, hHB22.7, and RFB4 are provided below. The CDRs in each heavy chain or light chain are underlined.

(hLL2-light chain variable region)
SEQ ID NO: 1
DIQLTQSPSSLSASVGDRVTMSCKSSQSVLYSANHKNYLAWYQQKPGKAP
KLLIYWASTRESGVPSRFSGSGSGTDFTFTISSLQPEDIATYYCHQYLSS
WTFGGGTKLEIK
(hLL2 heavy chain variable region)
SEQ ID NO: 2
QVQLVQSGAEVKKPGSSVKVSCKASGYTFTSYWLHWVRQAPGQGLEWIGY
INPRNDYTEYNQNFKDKATITADESTNTAYMELSSLRSEDTAFYFCARRD
ITTFYWGQGTTVTVSS
(h10F4 light chain variable region)
SEQ ID NO: 3
DIQMTQSPSSLSASVGDRVTITCRSSQSIVHSVGNTFLEWYQQKPGKAPK
LLIYKVSNRFSGVPSRFSGSGSGTDFTLTISSLQPEDFATYYCFQGSQFP
YTFGQGTKVEIK
(h10F4 heavy chain variable region)
SEQ ID NO: 4
EVQLVESGGGLVQPGGSLRLSCAASGYEFSRSWMNWVRQAPGKGLEWVGR
IYPGDGDTNYSGKFKGRFTISADTSKNTAYLQMNSLRAEDTAVYYCARDG
SSWDWYFDVWGQGTLVTVSS
(g5/44 light chain variable region)
SEQ ID NO: 5
DVQVTQSPSSLSASVGDRVTITCRSSQSLANSYGNTFLSWYLHKPGKAPQ
LLIYGISNRFSGVPDRFSGSGSGTDFTLTISSLQPEDFATYYCLQGTHQP
YTFGQGTKVEIK
(g5/44 heavy chain variable region)
SEQ ID NO: 6
EVQLVQSGAEVKKPGASVKVSCKASGYRFTNYWIHWVRQAPGQGLEWIGG
INPGNNYATYRRKFQGRVTMTADTSTSTVYMELSSLRSEDTAVYYCTREG
YGNYGAWFAYWGQGTLVTVSS
(hHB22.7 light chain variable region)
SEQ ID NO: 7
DIVMTQSPSSLSASVGDRVTITCKASQSVTNDVAWYQQKPGKAPKLLIYY
ASNRYTGVPSRFSGSGSGTDFTLTISSLQPEDFATYYCQQDYRSPWTFGG
GTKVEIK
(hHB22.7 heavy chain variable region)
SEQ ID NO: 8
QVQLEESGGGVVRPGRSLRLSCAASGFTFDDYGVNWIRQAPGKGLEWVT
IIWGDGRTDYNSALKSRFTVSRNNSNNTLSLQMNSLTTEDTAVYYCVRA
PGNRAMEYWGQGVLVTVSS
(RFB4 light chain variable region)
SEQ ID NO: 9
DIQMTQTTSSLSASLGDRVTISCRASQDISNYLNWYQQKPDGTVKLLIY
YTSILHSGVPSRFSGSGSGTDYSLTISNLEQEDFATYFCQQGNTLPWTF
GGGTKLEIK
(RFB4 heavy chain variable region)
SEQ ID NO: 10
EVQLVESGGGLVKPGGSLKLSCAASGFAFSIYDMSWVRQTPEKRLEWVA
YISSGGGTTYYPDTVKGRFTISRDNAKNTLYLQMSSLKSEDTAMYYCAR
HSGYGSSYGVLFAYWGQGTLVTVSA

The amino acid sequences of the light chain comprising human kappa constant domain, the heavy chain comprising human IgG1 constant domain, and heavy chain comprising human IgG4p constant domain are provided below.

(human kappa light chain constant domain)
SEQ ID NO: 11
RTVAAPSVFIFPPSDEQLKSGTASVVCLLNNFYPREAKVQWKVDNALQSG
NSQESVTEQDSKDSTYSLSSTLTLSKADYEKHKVYACEVTHQGLSSPVTK
SFNRGEC
(human IgG1 heavy chain constant domain)
SEQ ID NO: 12
ASTKGPSVFPLAPSSKSTSGGTAALGCLVKDYFPEPVTVSWNSGALTSGV
HTFPAVLQSSGLYSLSSVVTVPSSSLGTQTYICNVNHKPSNTKVDKKVEP
KSCDKTHTCPPCPAPELLGGPSVFLFPPKPKDTLMISRTPEVTCVVVDVS
HEDPEVKFNWYVDGVEVHNAKTKPREEQYNSTYRVVSVLTVLHQDWLNGK
EYKCKVSNKALPAPIEKTISKAKGQPREPQVYTLPPSRDELTKNQVSLTC
LVKGFYPSDIAVEWESNGQPENNYKTTPPVLDSDGSFFLYSKLTVDKSRW
QQGNVFSCSVMHEALHNHYTQKSLSLSPGK
(human IgG4p heavy chain constant domain)
SEQ ID NO: 13
ASTKGPSVFPLAPCSRSTSESTAALGCLVKDYFPEPVTVSWNSGALTSGV
HTFPAVLQSSGLYSLSSVVTVPSSSLGTKTYTCNVDHKPSNTKVDKRVES
KYGPPCPPCPAPEFLGGPSVFLFPPKPKDTLMISRTPEVTCVVVDVSQED
PEVQFNWYVDGVEVHNAKTKPREEQFNSTYRVVSVLTVLHQDWLNGKEYK
CKVSNKGLPSSIEKTISKAKGQPREPQVYTLPPSQEEMTKNQVSLTCLVK
GFYPSDIAVEWESNGQPENNYKTTPPVLDSDGSFFLYSRLTVDKSRWQEG
NVFSCSVMHEALHNHYTQKSLSLSLGK
(hLL2 light chain)
SEQ ID NO: 45
DIQLTQSPSSLSASVGDRVTMSCKSSQSVLYSANHKNYLAWYQQKPGKAP
KLLIYWASTRESGVPSRFSGSGSGTDFTFTISSLQPEDIATYYCHQYLSS
WTFGGGTKLEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(hLL2 heavy chain comprising human IgG1 constant
domain)
SEQ ID NO: 46
QVQLVQSGAEVKKPGSSVKVSCKASGYTFTSYWLHWVRQAPGQGLEWIGY
INPRNDYTEYNQNFKDKATITADESTNTAYMELSSLRSEDTAFYFCARRD
ITTFYWGQGTTVTVSSASTKGPSVFPLAPSSKSTSGGTAALGCLVKDYFP
EPVTVSWNSGALTSGVHTFPAVLQSSGLYSLSSVVTVPSSSLGTQTYICN
VNHKPSNTKVDKKVEPKSCDKTHTCPPCPAPELLGGPSVFLFPPKPKDTL
MISRTPEVTCVVVDVSHEDPEVKFNWYVDGVEVHNAKTKPREEQYNSTYR
VVSVLTVLHQDWLNGKEYKCKVSNKALPAPIEKTISKAKGQPREPQVYTL
PPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
(hLL2 heavy chain comprising human IgG4p constant
domain)
SEQ ID NO: 47
QVQLVQSGAEVKKPGSSVKVSCKASGYTFTSYWLHWVRQAPGQGLEWIGY
INPRNDYTEYNQNFKDKATITADESTNTAYMELSSLRSEDTAFYFCARRD
ITTFYWGQGTTVTVSSASTKGPSVFPLAPCSRSTSESTAALGCLVKDYFP
EPVTVSWNSGALTSGVHTFPAVLQSSGLYSLSSVVTVPSSSLGTKTYTCN
VDHKPSNTKVDKRVESKYGPPCPPCPAPEFLGGPSVFLFPPKPKDTLMIS
RTPEVTCVVVDVSQEDPEVQFNWYVDGVEVHNAKTKPREEQFNSTYRVVS
VLTVLHQDWLNGKEYKCKVSNKGLPSSIEKTISKAKGQPREPQVYTLPPS
QEEMTKNQVSLTCLVKGFYPSDIAVEWESNGQPENNYKTTPPVLDSDGSF
FLYSRLTVDKSRWQEGNVFSCSVMHEALHNHYTQKSLSLSLGK
(h10F4 light chain)
SEQ ID NO: 48
DIQMTQSPSSLSASVGDRVTITCRSSQSIVHSVGNTFLEWYQQKPGKAPK
LLIYKVSNRFSGVPSRFSGSGSGTDFTLTISSLQPEDFATYYCFQGSQFP
YTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(h10F4 heavy chain comprising human IgG1 constant
domain)
SEQ ID NO: 49
EVQLVESGGGLVQPGGSLRLSCAASGYEFSRSWMNWVRQAPGKGLEWVGR
IYPGDGDTNYSGKFKGRFTISADTSKNTAYLQMNSLRAEDTAVYYCARDG
SSWDWYFDVWGQGTLVTVSSASTKGPSVFPLAPSSKSTSGGTAALGCLVK
DYFPEPVTVSWNSGALTSGVHTFPAVLQSSGLYSLSSVVTVPSSSLGTQT
YICNVNHKPSNTKVDKKVEPKSCDKTHTCPPCPAPELLGGPSVFLFPPKP
KDTLMISRTPEVTCVVVDVSHEDPEVKFNWYVDGVEVHNAKTKPREEQYN
STYRVVSVLTVLHQDWLNGKEYKCKVSNKALPAPIEKTISKAKGQPREPQ
VYTLPPSRDELTKNQVSLTCLVKGFYPSDIAVEWESNGQPENNYKTTPPV
LDSDGSFFLYSKLTVDKSRWQQGNVFSCSVMHEALHNHYTQKSLSLSPGK
(h10F4 heavy chain comprising human IgG4p constant
domain)
SEQ ID NO: 50
EVQLVESGGGLVQPGGSLRLSCAASGYEFSRSWMNWVRQAPGKGLEWVGR
IYPGDGDTNYSGKFKGRFTISADTSKNTAYLQMNSLRAEDTAVYYCARDG
SSWDWYFDVWGQGTLVTVSSASTKGPSVFPLAPCSRSTSESTAALGCLVK
DYFPEPVTVSWNSGALTSGVHTFPAVLQSSGLYSLSSVVTVPSSSLGTKT
YTCNVDHKPSNTKVDKRVESKYGPPCPPCPAPEFLGGPSVFLFPPKPKDT
LMISRTPEVTCVVVDVSQEDPEVQFNWYVDGVEVHNAKTKPREEQFNSTY
RVVSVLTVLHQDWLNGKEYKCKVSNKGLPSSIEKTISKAKGQPREPQVYT
LPPSQEEMTKNQVSLTCLVKGFYPSDIAVEWESNGQPENNYKTTPPVLDS
DGSFFLYSRLTVDKSRWQEGNVFSCSVMHEALHNHYTQKSLSLSLGK
(g5/44 light chain)
SEQ ID NO: 51
DVQVTQSPSSLSASVGDRVTITCRSSQSLANSYGNTFLSWYLHKPGKAPQ
LLIYGISNRFSGVPDRFSGSGSGTDFTLTISSLQPEDFATYYCLQGTHQP
YTFGQGTKVEIKRTVAAPSVFIFPPSDEQLKSGTASVVCLLNNFYPREAK
VQWKVDNALQSGNSQESVTEQDSKDSTYSLSSTLTLSKADYEKHKVYACE
VTHQGLSSPVTKSFNRGEC
(g5/44 heavy chain comprising human IgG1 constant
domain)
SEQ ID NO: 52
EVQLVQSGAEVKKPGASVKVSCKASGYRFTNYWIHWVRQAPGQGLEWIGG
INPGNNYATYRRKFQGRVTMTADTSTSTVYMELSSLRSEDTAVYYCTREG
YGNYGAWFAYWGQGTLVTVSSASTKGPSVFPLAPSSKSTSGGTAALGCLV
KDYFPEPVTVSWNSGALTSGVHTFPAVLQSSGLYSLSSVVTVPSSSLGTQ
TYICNVNHKPSNTKVDKKVEPKSCDKTHTCPPCPAPELLGGPSVFLFPPK
PKDTLMISRTPEVTCVVVDVSHEDPEVKFNWYVDGVEVHNAKTKPREEQY
NSTYRVVSVLTVLHQDWLNGKEYKCKVSNKALPAPIEKTISKAKGQPREP
QVYTLPPSRDELTKNQVSLTCLVKGFYPSDIAVEWESNGQPENNYKTTPP
VLDSDGSFFLYSKLTVDKSRWQQGNVFSCSVMHEALHNHYTQKSLSLSPG
K
(g5/44 heavy chain comprising human IgG4p constant
domain)
SEQ ID NO: 53
EVQLVQSGAEVKKPGASVKVSCKASGYRFTNYWIHWVRQAPGQGLEWIGG
INPGNNYATYRRKFQGRVTMTADTSTSTVYMELSSLRSEDTAVYYCTREG
YGNYGAWFAYWGQGTLVTVSSASTKGPSVFPLAPCSRSTSESTAALGCLV
KDYFPEPVTVSWNSGALTSGVHTFPAVLQSSGLYSLSSVVTVPSSSLGTK
TYTCNVDHKPSNTKVDKRVESKYGPPCPPCPAPEFLGGPSVFLFPPKPKD
TLMISRTPEVTCVVVDVSQEDPEVQFNWYVDGVEVHNAKTKPREEQFNST
YRVVSVLTVLHQDWLNGKEYKCKVSNKGLPSSIEKTISKAKGQPREPQVY
TLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
(hHB22.7 light chain)
SEQ ID NO: 54
DIVMTQSPSSLSASVGDRVTITCKASQSVTNDVAWYQQKPGKAPKLLIYY
ASNRYTGVPSRFSGSGSGTDFTLTISSLQPEDFATYYCQQDYRSPWTFGG
GTKVEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQG
LSSPVTKSFNRGEC
(hHB22.7 heavy chain comprising human IgG1
constant domain)
SEQ ID NO: 55
QVQLEESGGGVVRPGRSLRLSCAASGFTFDDYGVNWIRQAPGKGLEWVTI
IWGDGRTDYNSALKSRFTVSRNNSNNTLSLQMNSLTTEDTAVYYCVRAPG
NRAMEYWGQGVLVTVSSASTKGPSVFPLAPSSKSTSGGTAALGCLVKDYF
PEPVTVSWNSGALTSGVHTFPAVLQSSGLYSLSSVVTVPSSSLGTQTYIC
NVNHKPSNTKVDKKVEPKSCDKTHTCPPCPAPELLGGPSVFLFPPKPKDT
LMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYT
LPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
(hHB22.7 heavy chain comprising human IgG4p
constant domain)
SEQ ID NO: 56
QVQLEESGGGVVRPGRSLRLSCAASGFTFDDYGVNWIRQAPGKGLEWVTI
IWGDGRTDYNSALKSRFTVSRNNSNNTLSLQMNSLTTEDTAVYYCVRAPG
NRAMEYWGQGVLVTVSSASTKGPSVFPLAPCSRSTSESTAALGCLVKDYF
PEPVTVSWNSGALTSGVHTFPAVLQSSGLYSLSSVVTVPSSSLGTKTYTC
NVDHKPSNTKVDKRVESKYGPPCPPCPAPEFLGGPSVFLFPPKPKDTLMI
SRTPEVTCVVVDVSQEDPEVQFNWYVDGVEVHNAKTKPREEQFNSTYRVV
SVLTVLHQDWLNGKEYKCKVSNKGLPSSIEKTISKAKGQPREPQVYTLPP
SQEEMTKNQVSLTCLVKGFYPSDIAVEWESNGQPENNYKTTPPVLDSDGS
FFLYSRLTVDKSRWQEGNVFSCSVMHEALHNHYTQKSLSLSLGK

The CDRs of anti-CD22 antibodies hLL2, h10F4, g5/44, hHB22.7, and RFB4 are provided in Tables 2 and 3 below.

TABLE 2
Amino Acid Sequences of CDRs of anti-CD22
Antibodies hLL2, h10F4, and g5/44

| | hLL2 | h10F4 | g5/44 |
|---|---|---|---|
| CDR- | SYWLH | RSWMN | NYWIH |
| H1 | (SEQ ID NO: 15) | (SEQ ID NO: 21) | (SEQ ID NO: 27) |
| CDR- | YINPRNDYTEYNQ | RIYPGDGDTNYSG | GINPGNNYATYRR |
| H2 | NFKD | KFKG | KFQG |
| | (SEQ ID NO: 16) | (SEQ ID NO: 22) | (SEQ ID NO: 28) |
| CDR- | RDITTFY | DGSSWDWYFDV | EGYGNYGAWFAY |
| H3 | (SEQ ID NO: 17) | (SEQ ID NO: 23) | (SEQ ID NO: 29) |
| CDR- | KSSQSVLYSANHK | RSSQSIVHSVGNT | RSSQSLANSYGNT |
| L1 | NYLA | FLE | FLS |
| | (SEQ ID NO: 18) | (SEQ ID NO: 24) | (SEQ ID NO: 30) |
| CDR- | WASTRES | KVSNRFS | GISNRFS |
| L2 | (SEQ ID NO: 19) | (SEQ ID NO: 25) | (SEQ ID NO: 31) |
| CDR- | HQYLSSWT | FQGSQFPYT | LQGTHQPYT |
| L3 | (SEQ ID NO: 20) | (SEQ ID NO: 26) | (SEQ ID NO: 32) |

TABLE 3
Amino Acid Sequences of CDRs of anti-CD22
Antibodies hHB22.7 and RFB4

| | hHB22.7 | RFB4 |
|---|---|---|
| CDR-H1 | DYGVN | IYDMS |
| | (SEQ ID NO: 33) | (SEQ ID NO: 39) |
| CDR-H2 | IIWGDGRTDYNSALKS | YISSGGGTTYYPDTVKG |
| | (SEQ ID NO: 34) | (SEQ ID NO: 40) |
| CDR-H3 | APGNRAMEY | HSGYGSSYGVLFAY |
| | (SEQ ID NO: 35) | (SEQ ID NO: 41) |
| CDR-L1 | KASQSVTNDVA | RASQDISNYLN |
| | (SEQ ID NO: 36) | (SEQ ID NO: 42) |
| CDR-L2 | YASNRYT | YTSILHS |
| | (SEQ ID NO: 37) | (SEQ ID NO: 43) |
| CDR-L3 | QQDYRSPWT | QQGNTLPWT |
| | (SEQ ID NO: 38) | (SEQ ID NO: 44) |

In some embodiments, the anti-CD22 antibody is antibody hLL2, h10F4, g5/44, or hHB22.7, or an antibody derived from any of these antibodies. In some embodiments, the anti-CD22 antibody is an antibody derived from antibody RFB4, such as a humanized or chimeric antibody. The light chain and heavy chain variable sequences of antibody hLL2, h10F4, g5/44, hHB22.7, and RFB4 are set forth above in Table 1. Examples of humanized RFB4 scFv (single chain fragment of the variable regions) have been described. See, for example, Krauss J. et al., Protein Engineering, 16(10):753-759, 2003; and the sequences corresponding to humanized RFB4 scFvs and fragments thereof (such as heavy chain variable region, light chain variable region, etc.) described in Krauss J. et al., are incorporated herein by reference. The heavy chain variable region of any one of the humanized RFB4 scFvs described by Krauss et al. may be combined with a human heavy chain constant region (such as human IgG1 or human IgG4p, including SEQ ID NO:12 or SEQ ID No:13) to form a humanized heavy chain of RFB4. The light chain variable region of any one of the humanized RFB4 scFvs described by Krauss et al. may be combined with a human light chain constant region (such as human kappa light chain, including SEQ ID NO:11) to form a humanized light chain of RFB4. In some embodiments, the anti-CD22 antibody comprises one, two, or three HVRs (or CDRs) from a light chain or a heavy chain of the antibody hLL2, h10F4, g5/44, hHB22.7, or RFB4 (or an antibody derived from any one of these antibodies, including the humanized RFB4 scFvs described by Krauss et al.), such as the HVR (or CDR) sequences set forth above in Tables 2 and 3. In some embodiments, the anti-CD22 antibody comprises one, two, or three HVRs (or CDRs) from a light chain and a heavy chain of the antibody hLL2, h10F4, g5/44, hHB22.7, or RFB4 (or an antibody derived from any one of these antibodies, including the humanized RFB4 scFvs described by Krauss et al.), such as the HVR (or CDR) sequences set forth above in Tables 2 and 3. In some embodiments, the anti-CD22 antibody comprises a fragment or a region of the antibody hLL2, h10F4, g5/44, hHB22.7, or RFB4 (including the humanized RFB4 scFvs described by Krauss et al. and antibodies derived therefrom). In one embodiment, the fragment comprises a light chain variable region of the antibody hLL2, h10F4, g5/44, hHB22.7, or RFB4 (including the humanized RFB4 scFvs described by Krauss et al.). In another embodiment, the fragment comprises a heavy chain variable region of the antibody hLL2, h10F4, g5/44, hHB22.7, or RFB4 (including the humanized RFB4 scFvs described by Krauss et al.). In some embodiments, the anti-CD22 antibody comprises light chain and heavy chain variable regions of antibody hLL2, h10F4, g5/44, hHB22.7, or RFB4 (including the humanized RFB4 scFvs described by Krauss et al.). In yet another embodiment, the fragment comprises one, two, or three HVRs (or CDRs) from a light chain or a heavy chain of the antibody hLL2, h10F4, g5/44, hHB22.7, or RFB4 (including light chains or heavy chains derived from the humanized RFB4 scFv described by Krauss et al.). In yet another embodiment, the fragment comprises one, two, or three HVRs (or CDRs) from a light chain and a heavy chain of the antibody hLL2, h10F4, g5/44, hHB22.7, or RFB4 (including light chains or heavy chains derived from the humanized RFB4 scFv described by Krauss et al.). In some embodiments, the one or more HVRs (or CDRs) derived from antibody hLL2, h10F4, g5/44, hHB22.7, or RFB4 are at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to at least one, at least two, at least three, at least four, at least five, or at least six HVRs (or CDRs) of hLL2, h10F4, g5/44, hHB22.7, or RFB4 (including the humanized RFB4 scFvs described by Krauss et al.).

In some embodiments, the anti-CD22 antibody comprises a heavy chain variable region comprising one, two or three HVRs (or CDRs) from SEQ ID NO:2 and/or a light chain variable region comprising one, two or three HVRs (or CDRs) from SEQ ID NO:1. In some embodiments, the antibody comprises a heavy chain variable region comprising the three HVRs (or CDRs) from SEQ ID NO:2 and/or a light chain variable region comprising the three HVRs (or CDRs) from SEQ ID NO:1. In some embodiments, the anti-CD22 antibody comprises a heavy chain variable region comprising one, two or three HVRs (or CDRs) from SEQ ID NO:2. In some embodiments, the anti-CD22 antibody comprises a light chain variable region comprising one, two or three HVRs (or CDRs) from SEQ ID NO:1. In some embodiments, the anti-CD22 antibody comprises a heavy chain variable region comprising one, two or three HVRs (or CDRs) from SEQ ID NO:2 and a light chain variable region comprising one, two or three HVRs (or CDRs) from SEQ ID NO:1. In some embodiments, the anti-CD22 antibody comprises a heavy chain variable region comprising one, two or three HVRs (or CDRs) selected from SEQ ID NOs: 15, 16, and 17. In some embodiments, the anti-CD22 antibody comprises a light chain variable region comprising one, two or three HVRs (or CDRs) selected from SEQ ID NOs: 18, 19, and 20. In some embodiments, the anti-CD22 antibody comprises a heavy chain variable region comprising one, two or three HVRs (or CDRs) selected from SEQ ID NOs: 15, 16, and 17; and a light chain variable region comprising one, two or three HVRs (or CDRs) selected from SEQ ID NOs: 18, 19, and 20. In some embodiments, the anti-CD22 antibody comprises a heavy chain variable region comprising the three HVRs (or CDRs) from SEQ ID NO:2. In some embodiments, the anti-CD22 antibody comprises a light chain variable region comprising the three HVRs (or CDRs) from SEQ ID NO:1. In some embodiments, the anti-CD22 antibody comprises a heavy chain variable region comprising the three HVRs (or CDRs) from SEQ ID NO:2, and a light chain variable region comprising the three HVRs (or CDRs) from SEQ ID NO:1. In some embodiments, the anti-CD22 antibody comprises a heavy chain variable region comprising the heavy chain variable region (VH) CDR1 sequence set forth in SEQ ID NO:15, the VH CDR2 sequence set forth in SEQ ID NO:16, and the VH CDR3 sequence set forth in SEQ ID NO:17. In some embodiments, the anti-CD22 antibody comprises a light chain variable region comprising the light chain variable region (VL) CDR1 sequence set forth in SEQ ID NO:18, the VL CDR2 sequence set forth in SEQ ID NO:19, and the VL CDR3 sequence set forth in SEQ ID NO:20. In some embodiments, the anti-CD22 antibody comprises a heavy chain variable region comprising the VH CDR1 sequence set forth in SEQ ID NO:15, the VH CDR2 sequence set forth in SEQ ID NO:16, and the VH CDR3 sequence set forth in SEQ ID NO:17; and a light chain variable region comprising the VL CDR1 sequence set forth in SEQ ID NO:18, the VL CDR2 sequence set forth in SEQ ID NO:19, and the VL CDR3 sequence set forth in SEQ ID NO:20.

In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:2, and/or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:1. In some embodiments, the antibody comprises a heavy chain variable region comprising amino acid sequence of SEQ ID NO:2 and/or a light chain variable region comprising amino acid sequence of SEQ ID NO:1. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:2, or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:1. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:2, and a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:1. In some embodiments, the anti-CD22 antibody comprises a heavy chain variable region comprising amino acid sequence of SEQ ID NO:2. In some embodiments, the anti-CD22 antibody comprises a light chain variable region comprising amino acid sequence of SEQ ID NO:1. In some embodiments, the anti-CD22 antibody comprises a heavy chain variable region comprising amino acid sequence of SEQ ID NO:2, and a light chain variable region comprising amino acid sequence of SEQ ID NO:1.

In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:46 or SEQ ID NO: 47, and/or a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:45. In some embodiments, the antibody comprises a heavy chain comprising amino acid sequence of SEQ ID NO:46 or SEQ ID NO: 47 and/or a light chain comprising amino acid sequence of SEQ ID NO:45. In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:46 or SEQ ID NO: 47, or a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:45. In some embodiments, the anti-CD22 antibody comprises a heavy chain variable region comprising amino acid sequence of SEQ ID NO:2, and a light chain variable region comprising amino acid sequence of SEQ ID NO:1. In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:46 or SEQ ID NO: 47, and a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:45. In some embodiments, the antibody comprises a heavy chain comprising amino acid sequence of SEQ ID NO:46 or SEQ ID NO: 47. In some embodiments, the antibody comprises a light chain comprising amino acid sequence of SEQ ID NO:45. In some embodiments, the antibody comprises a heavy chain comprising amino acid sequence of SEQ ID NO:46 or SEQ ID NO: 47 and a light chain comprising amino acid sequence of SEQ ID NO:45. In some embodiments, the antibody is humanized antibody.

In some embodiments, the anti-CD22 antibody comprises a heavy chain variable region comprising one, two or three HVRs (or CDRs) from SEQ ID NO:4 and/or a light chain variable region comprising one, two or three HVRs (or CDRs) from SEQ ID NO:3. In some embodiments, the antibody comprises a heavy chain variable region comprising the three HVRs (or CDRs) from SEQ ID NO:4 and/or a light chain variable region comprising the three HVRs (or CDRs) from SEQ ID NO:3. In some embodiments, the anti-CD22 antibody comprises a heavy chain variable region comprising one, two or three HVRs (or CDRs) from SEQ ID NO:4. In some embodiments, the anti-CD22 antibody comprises a light chain variable region comprising one, two or three HVRs (or CDRs) from SEQ ID NO:3. In some embodiments, the anti-CD22 antibody comprises a heavy chain variable region comprising one, two or three HVRs (or CDRs) from SEQ ID NO:4 and a light chain variable region comprising one, two or three HVRs (or CDRs) from SEQ ID NO:3. In some embodiments, the anti-CD22 antibody comprises a heavy chain variable region comprising one, two or three HVRs (or CDRs) selected from SEQ ID NOs: 21, 22 and 23. In some embodiments, the anti-CD22 antibody comprises a light chain variable region comprising one, two or three HVRs (or CDRs) selected from SEQ ID NOs: 24, 25 and 26. In some embodiments, the anti-CD22 antibody comprises a heavy chain variable region comprising one, two or three HVRs (or CDRs) selected from SEQ ID NOs: 21, 22 and 23 and a light chain variable region comprising one, two or three HVRs (or CDRs) selected from SEQ ID NOs: 24, 25 and 26. In some embodiments, the anti-CD22 antibody comprises a heavy chain variable region comprising the three HVRs (or CDRs) from SEQ ID NO:4. In some embodiments, the anti-CD22 antibody comprises a light chain variable region comprising the three HVRs (or CDRs) from SEQ ID NO:3. In some embodiments, the anti-CD22 antibody comprises a heavy chain variable region comprising the three HVRs (or CDRs) from SEQ ID NO:4 and a light chain variable region comprising the three HVRs (or CDRs) from SEQ ID NO:3. In some embodiments, the anti-CD22 antibody comprises a heavy chain variable region comprising a heavy chain variable region comprising the VH CDR1 sequence set forth in SEQ ID NO:21, the VH CDR2 sequence set forth in SEQ ID NO:22, and the VH CDR3 sequence set forth in SEQ ID NO:23. In some embodiments, the anti-CD22 antibody comprises a light chain variable region comprising the VL CDR1 sequence set forth in SEQ ID NO:24, the VL CDR2 sequence set forth in SEQ ID NO:25, and the VL CDR3 sequence set forth in SEQ ID NO:26. In some embodiments, the anti-CD22 antibody comprises a heavy chain variable region comprising the VH CDR1 sequence set forth in SEQ ID NO:21, the VH CDR2 sequence set forth in SEQ ID NO:22, and the VH CDR3 sequence set forth in SEQ ID NO:23; and a light chain variable region comprising the VL CDR1 sequence set forth in SEQ ID NO:24, the VL CDR2 sequence set forth in SEQ ID NO:25, and the VL CDR3 sequence set forth in SEQ ID NO:26.

In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:4, and/or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:3. In some embodiments, the antibody comprises a heavy chain variable region comprising amino acid sequence of SEQ ID NO:4 and/or a light chain variable region comprising amino acid sequence of SEQ ID NO:3. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:4, or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:3. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:4, and a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:3. In some embodiments, the antibody comprises a heavy chain variable region comprising amino acid sequence of SEQ ID NO:4. In some embodiments, the antibody comprises a light chain variable region comprising amino acid sequence of SEQ ID NO:3. In some embodiments, the antibody comprises a heavy chain variable region comprising amino acid sequence of SEQ ID NO:4 and a light chain variable region comprising amino acid sequence of SEQ ID NO:3.

In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:49 or SEQ ID NO: 50, and/or a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:48. In some embodiments, the antibody comprises a heavy chain comprising amino acid sequence of SEQ ID NO:49 or SEQ ID NO:50 and/or a light chain variable region comprising amino acid sequence of SEQ ID NO:48. In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:49 or SEQ ID NO: 50, or a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:48. In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:49 or SEQ ID NO: 50, and a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:48. In some embodiments, the antibody comprises a heavy chain comprising amino acid sequence of SEQ ID NO:49 or SEQ ID NO:50. In some embodiments, the antibody comprises a light chain variable region comprising amino acid sequence of SEQ ID NO:48. In some embodiments, the antibody comprises a heavy chain comprising amino acid sequence of SEQ ID NO:49 or SEQ ID NO:50, and a light chain variable region comprising amino acid sequence of SEQ ID NO:48. In some embodiments, the antibody is humanized antibody.

In some embodiments, the anti-CD22 antibody comprises a heavy chain variable region comprising one, two or three HVRs (or CDRs) from SEQ ID NO:6 and/or a light chain variable region comprising one, two or three HVRs (or CDRs) from SEQ ID NO:5. In some embodiments, the antibody comprises a heavy chain variable region comprising the three HVRs (or CDRs) from SEQ ID NO:6 and/or a light chain variable region comprising the three HVRs (or CDRs) from SEQ ID NO:5. In some embodiments, the anti-CD22 antibody comprises a heavy chain variable region comprising one, two or three HVRs (or CDRs) from SEQ ID NO:6. In some embodiments, the anti-CD22 antibody comprises a light chain variable region comprising one, two or three HVRs (or CDRs) from SEQ ID NO:5. In some embodiments, the anti-CD22 antibody comprises a heavy chain variable region comprising one, two or three HVRs (or CDRs) from SEQ ID NO:6 and a light chain variable region comprising one, two or three HVRs (or CDRs) from SEQ ID NO:5. In some embodiments, the anti-CD22 antibody comprises a heavy chain variable region comprising one, two or three HVRs (or CDRs) selected from SEQ ID NOs: 27, 28 and 29. In some embodiments, the anti-CD22 antibody comprises a light chain variable region comprising one, two or three HVRs (or CDRs) selected from SEQ ID NOs: 30, 31 and 32. In some embodiments, the anti-CD22 antibody comprises a heavy chain variable region comprising one, two or three HVRs (or CDRs) selected from SEQ ID NOs: 27, 28 and 29 and a light chain variable region comprising one, two or three HVRs (or CDRs) selected from SEQ ID NOs: 30, 31 and 32. In some embodiments, the antibody comprises a heavy chain variable region comprising the three HVRs (or CDRs) from SEQ ID NO:6. In some embodiments, the antibody comprises a light chain variable region comprising the three HVRs (or CDRs) from SEQ ID NO:5. In some embodiments, the antibody comprises a heavy chain variable region comprising the three HVRs (or CDRs) from SEQ ID NO:6 and a light chain variable region comprising the three HVRs (or CDRs) from SEQ ID NO:5. In some embodiments, the anti-CD22 antibody comprises a heavy chain variable region comprising the VH CDR1 sequence set forth in SEQ ID NO:27, the VH CDR2 sequence set forth in SEQ ID NO:28, and the VH CDR3 sequence set forth in SEQ ID NO:29. In some embodiments, the anti-CD22 antibody comprises a light chain variable region comprising the VL CDR1 sequence set forth in SEQ ID NO:30, the VL CDR2 sequence set forth in SEQ ID NO:31, and the VL CDR3 sequence set forth in SEQ ID NO:32. In some embodiments, the anti-CD22 antibody comprises a heavy chain variable region comprising the VH CDR1 sequence set forth in SEQ ID NO:27, the VH CDR2 sequence set forth in SEQ ID NO:28, and the VH CDR3 sequence set forth in SEQ ID NO:29, and a light chain variable region comprising the VL CDR1 sequence set forth in SEQ ID NO:30, the VL CDR2 sequence set forth in SEQ ID NO:31, and the VL CDR3 sequence set forth in SEQ ID NO:32.

In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:6, and/or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:5. In some embodiments, the antibody comprises a heavy chain variable region comprising amino acid sequence of SEQ ID NO:6 and/or a light chain variable region comprising amino acid sequence of SEQ ID NO:5. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:6, or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:5. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:6, and a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:5. In some embodiments, the antibody comprises a heavy chain variable region comprising amino acid sequence of SEQ ID NO:6. In some embodiments, the antibody comprises a light chain variable region comprising amino acid sequence of SEQ ID NO:5. In some embodiments, the antibody comprises a heavy chain variable region comprising amino acid sequence of SEQ ID NO:6 and a light chain variable region comprising amino acid sequence of SEQ ID NO:5.

In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:52 or SEQ ID NO:53, and/or a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:51. In some embodiments, the antibody comprises a heavy chain comprising amino acid sequence of SEQ ID NO:52 or SEQ ID NO:53 and/or a light chain variable region comprising amino acid sequence of SEQ ID NO:51. In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:52 or SEQ ID NO:53, or a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:51. In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:52 or SEQ ID NO:53, and a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:51. In some embodiments, the antibody comprises a heavy chain comprising amino acid sequence of SEQ ID NO:52 or SEQ ID NO:53. In some embodiments, the antibody comprises a light chain variable region comprising amino acid sequence of SEQ ID NO:51. In some embodiments, the antibody comprises a heavy chain comprising amino acid sequence of SEQ ID NO:52 or SEQ ID NO:53 and a light chain variable region comprising amino acid sequence of SEQ ID NO:51. In some embodiments, the antibody is humanized antibody.

In some embodiments, the anti-CD22 antibody comprises a heavy chain variable region comprising one, two or three HVRs (or CDRs) from SEQ ID NO:8 and/or a light chain variable region comprising one, two or three HVRs (or CDRs) from SEQ ID NO:7. In some embodiments, the antibody comprises a heavy chain variable region comprising the three HVRs (or CDRs) from SEQ ID NO:8 and/or a light chain variable region comprising the three HVRs (or CDRs) from SEQ ID NO:7. In some embodiments, the anti-CD22 antibody comprises a heavy chain variable region comprising one, two or three HVRs (or CDRs) from SEQ ID NO:8. In some embodiments, the anti-CD22 antibody comprises a light chain variable region comprising one, two or three HVRs (or CDRs) from SEQ ID NO:7. In some embodiments, the anti-CD22 antibody comprises a heavy chain variable region comprising one, two or three HVRs (or CDRs) from SEQ ID NO:8 and a light chain variable region comprising one, two or three HVRs (or CDRs) from SEQ ID NO:7. In some embodiments, the anti-CD22 antibody comprises a heavy chain variable region comprising one, two or three HVRs (or CDRs) selected from SEQ ID NOs: 33, 34 and 35. In some embodiments, the anti-CD22 antibody comprises a light chain variable region comprising one, two or three HVRs (or CDRs) selected from SEQ ID NOs: 36, 37 and 38. In some embodiments, the anti-CD22 antibody comprises a heavy chain variable region comprising one, two or three HVRs (or CDRs) selected from SEQ ID NOs: 33, 34 and 35 and a light chain variable region comprising one, two or three HVRs (or CDRs) selected from SEQ ID NOs: 36, 37 and 38. In some embodiments, the antibody comprises a heavy chain variable region comprising the three HVRs (or CDRs) from SEQ ID NO:8. In some embodiments, the antibody comprises a light chain variable region comprising the three HVRs (or CDRs) from SEQ ID NO:7. In some embodiments, the antibody comprises a heavy chain variable region comprising the three HVRs (or CDRs) from SEQ ID NO:8 and a light chain variable region comprising the three HVRs (or CDRs) from SEQ ID NO:7. In some embodiments, the anti-CD22 antibody comprises a heavy chain variable region comprising the VH CDR1 sequence set forth in SEQ ID NO:33, the VH CDR2 sequence set forth in SEQ ID NO:34, and the VH CDR3 sequence set forth in SEQ ID NO:35. In some embodiments, the anti-CD22 antibody comprises a light chain variable region comprising the VL CDR1 sequence set forth in SEQ ID NO:36, the VL CDR2 sequence set forth in SEQ ID NO:37, and the VL CDR3 sequence set forth in SEQ ID NO:38. In some embodiments, the anti-CD22 antibody comprises a heavy chain variable region comprising the VH CDR1 sequence set forth in SEQ ID NO:33, the VH CDR2 sequence set forth in SEQ ID NO:34, and the VH CDR3 sequence set forth in SEQ ID NO:35; and a light chain variable region comprising the VL CDR1 sequence set forth in SEQ ID NO:36, the VL CDR2 sequence set forth in SEQ ID NO:37, and the VL CDR3 sequence set forth in SEQ ID NO:38.

In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:8, and/or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:7. In some embodiments, the antibody comprises a heavy chain variable region comprising amino acid sequence of SEQ ID NO:8 and/or a light chain variable region comprising amino acid sequence of SEQ ID NO:7. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:8, or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:7. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:8, and a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:7. In some embodiments, the antibody comprises a heavy chain variable region comprising amino acid sequence of SEQ ID NO:8. In some embodiments, the antibody comprises a light chain variable region comprising amino acid sequence of SEQ ID NO:7. In some embodiments, the antibody comprises a heavy chain variable region comprising amino acid sequence of SEQ ID NO:8 and a light chain variable region comprising amino acid sequence of SEQ ID NO:7.

In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:55 or SEQ ID NO:56, and/or a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:54. In some embodiments, the antibody comprises a heavy chain comprising amino acid sequence of SEQ ID NO:55 or SEQ ID NO:56 and/or a light chain comprising amino acid sequence of SEQ ID NO:54. In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:55 or SEQ ID NO:56, or a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:54. In some embodiments, the antibody comprises a heavy chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:55 or SEQ ID NO:56, and a light chain comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:54. In some embodiments, the antibody comprises a heavy chain comprising amino acid sequence of SEQ ID NO:55 or SEQ ID NO:56. In some embodiments, the antibody comprises a light chain comprising amino acid sequence of SEQ ID NO:54. In some embodiments, the antibody comprises a heavy chain comprising amino acid sequence of SEQ ID NO:55 or SEQ ID NO:56 and a light chain comprising amino acid sequence of SEQ ID NO:54. In some embodiments, the antibody is humanized antibody.

In some embodiments, the anti-CD22 antibody comprises a heavy chain variable region comprising one, two or three HVRs (or CDRs) from SEQ ID NO:10 and/or a light chain variable region comprising one, two or three HVRs (or CDRs) from SEQ ID NO:9. In some embodiments, the antibody comprises a heavy chain variable region comprising the three HVRs (or CDRs) from SEQ ID NO:10 and/or a light chain variable region comprising the three HVRs (or CDRs) from SEQ ID NO:9. In some embodiments, the anti-CD22 antibody comprises a heavy chain variable region comprising one, two or three HVRs (or CDRs) from SEQ ID NO:10. In some embodiments, the anti-CD22 antibody comprises a light chain variable region comprising one, two or three HVRs (or CDRs) from SEQ ID NO:9. In some embodiments, the anti-CD22 antibody comprises a heavy chain variable region comprising one, two or three HVRs (or CDRs) from SEQ ID NO:10 and a light chain variable region comprising one, two or three HVRs (or CDRs) from SEQ ID NO:9. In some embodiments, the anti-CD22 antibody comprises a heavy chain variable region comprising one, two or three HVRs (or CDRs) selected from SEQ ID NOs: 39, 40 and 41. In some embodiments, the anti-CD22 antibody comprises a light chain variable region comprising one, two or three HVRs (or CDRs) selected from SEQ ID NOs: 42, 43 and 44. In some embodiments, the anti-CD22 antibody comprises a heavy chain variable region comprising one, two or three HVRs (or CDRs) selected from SEQ ID NOs: 39, 40 and 41 and a light chain variable region comprising one, two or three HVRs (or CDRs) selected from SEQ ID NOs: 42, 43 and 44. In some embodiments, the antibody comprises a heavy chain variable region comprising the three HVRs (or CDRs) from SEQ ID NO:10. In some embodiments, the antibody comprises a light chain variable region comprising the three HVRs (or CDRs) from SEQ ID NO:9. In some embodiments, the antibody comprises a heavy chain variable region comprising the three HVRs (or CDRs) from SEQ ID NO:10 and a light chain variable region comprising the three HVRs (or CDRs) from SEQ ID NO:9. In some embodiments, the anti-CD22 antibody comprises a heavy chain variable region comprising the VH CDR1 sequence set forth in SEQ ID NO:39, the VH CDR2 sequence set forth in SEQ ID NO:40, and the VH CDR3 sequence set forth in SEQ ID NO:41. In some embodiments, the anti-CD22 antibody comprises a light chain variable region comprising the VL CDR1 sequence set forth in SEQ ID NO:42, the VL CDR2 sequence set forth in SEQ ID NO:43, and the VL CDR3 sequence set forth in SEQ ID NO:44. In some embodiments, the anti-CD22 antibody comprises a heavy chain variable region comprising the VH CDR1 sequence set forth in SEQ ID NO:39, the VH CDR2 sequence set forth in SEQ ID NO:40, and the VH CDR3 sequence set forth in SEQ ID NO:41; and a light chain variable region comprising the VL CDR1 sequence set forth in SEQ ID NO:42, the VL CDR2 sequence set forth in SEQ ID NO:43, and the VL CDR3 sequence set forth in SEQ ID NO:44.

In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:10, and/or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:9. In some embodiments, the antibody comprises a heavy chain variable region comprising amino acid sequence of SEQ ID NO:10 and/or a light chain variable region comprising amino acid sequence of SEQ ID NO:9. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:10, or a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:9. In some embodiments, the antibody comprises a heavy chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:10, and a light chain variable region comprising an amino acid sequence at least about 85%, at least about 86%, at least about 87%, at least about 88%, at least about 89%, at least about 90%, at least about 91%, at least about 92%, at least about 93%, at least about 94%, at least about 95%, at least about 96%, at least about 97%, at least about 98%, or at least about 99% identical to the sequence of SEQ ID NO:9. In some embodiments, the antibody comprises a heavy chain variable region comprising amino acid sequence of SEQ ID NO:10. In some embodiments, the antibody comprises a light chain variable region comprising amino acid sequence of SEQ ID NO:9. In some embodiments, the antibody comprises a heavy chain variable region comprising amino acid sequence of SEQ ID NO:10 and a light chain variable region comprising amino acid sequence of SEQ ID NO:9.

In some embodiments, the antibody is humanized antibody. In some embodiments, the antibody comprises a heavy chain comprising the amino acid sequence of SEQ ID NO: 12 or SEQ ID NO: 13 and/or a light chain comprising the amino acid sequence of SE ID NO: 11. In some embodiments, the antibody is humanized antibody. In some embodiments, the antibody comprises a heavy chain comprising the amino acid sequence of SEQ ID NO: 12 or SEQ ID NO: 13. In some embodiments, the antibody comprises a light chain comprising the amino acid sequence of SE ID NO: 11. In some embodiments, the antibody comprises a heavy chain comprising the amino acid sequence of SEQ ID NO: 12 or SEQ ID NO: 13 and a light chain comprising the amino acid sequence of SE ID NO: 11. In some embodiments, the antibody is a chimeric antibody. In some embodiments, the antibody is a humanized antibody. In some embodiments, the antibody is a humanized antibody derived from any one of the humanized scFvs described in Krauss J. et al., Protein Engineering, 16(10): 753-759, 2003.

In some embodiments, an anti-CD22 antibody included or used in a targeting moiety described herein (or an antibody targeting moiety) specifically binds to a CD22 (such as a human CD22) expressed by cancer cells (e.g., CD22-positive hematological malignancy (such as B cell lymphoma, diffuse large B-cell lymphoma, Burkitt lymphoma, non-Hodgkin's lymphoma, follicular lymphoma, and acute lymphoblastic leukemia (ALL)).

As used herein, “percent (%) amino acid sequence identity” and “homology” with respect to a sequence refers to the percentage of amino acid residues in a candidate sequence that are identical with the amino acid residues in the specific sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or MEGALIGN™ (DNASTAR) software. Those skilled in the art can determine appropriate parameters for measuring alignment, including any algorithms needed to achieve maximal alignment over the full length of the sequences being compared.

In some embodiments, a CDR described herein is Kabat CDR, Chothia CDR, or contact CDR. In some embodiments, the CDR is a Kabat CDR. In some embodiments, the CDR is a Chothia CDR. In other embodiments, the CDR is a combination of a Kabat and a Chothia CDR (also termed “combined CDR” or “extended CDR”). In other words, for any given embodiment containing more than one CDR, the CDRs may be any of Kabat, Chothia, and/or combined. Methods of determining CDRs are known in the field.

A variable region of an antibody refers to the variable region of the antibody light chain or the variable region of the antibody heavy chain, either alone or in combination. Generally, the variable region(s) mediate antigen binding and define specificity of a particular antibody for its particular antigen. The variable regions may have relatively invariant stretches called framework regions (FRs) (e.g., FR of 15-30 amino acids) separated by shorter regions of extreme variability called “hypervariable regions” (“HVR”) (e.g., HVRs that are each 9-12 amino acids long). In some embodiments, the variable domains of native heavy and light chains each comprise four FRs, largely adopting a beta-sheet configuration, connected by three hypervariable regions, which form loops connecting, and in some cases forming part of, the beta-sheet structure. The hypervariable regions in each chain may be held together in close proximity by the FRs and, with the hypervariable regions from the other chain, contribute to the formation of the antigen-binding site of antibodies (see Kabat et al., Sequences of Proteins of Immunological Interest. 5th Ed. Public Health Service, National Institutes of Health, Bethesda, Md. (1991)). The constant domains may not be involved directly in binding an antibody to an antigen, but may exhibit various effector functions, such as participation of the antibody in antibody dependent cellular cytotoxicity (ADCC). A constant region of an antibody refers to the constant region of the antibody light chain or the constant region of the antibody heavy chain, either alone or in combination. A constant region of an antibody generally provides structural stability and other biological functions such as antibody chain association, secretion, transplacental mobility, and complement binding, but is not involved with binding to the antigen. The amino acid sequence and corresponding exon sequences in the genes of the constant region will be dependent upon the species from which it is derived; however, variations in the amino acid sequence leading to allotypes will be relatively limited for particular constant regions within a species. The variable region of each chain is joined to the constant region by a linking polypeptide sequence. The linkage sequence is coded by a “J” sequence in the light chain gene, and a combination of a “D” sequence and a “J” sequence in the heavy chain gene.

The term “hypervariable region” (“HVR”) when used herein refers to the amino acid residues of an antibody which are responsible for antigen-binding. The hypervariable region generally comprises amino acid residues from a “complementarity determining region” or “CDR” (e.g. around about residues 24-34 (L1), 50-56 (L2) and 89-97 (L3) in the VL, and around about 31-35B (H1), 50-65 (H2) and 95-102 (H3) in the VH (in one embodiment, H1 is around about 31-35); Kabat et al., Sequences of Proteins of Immunological Interest. 5th Ed. Public Health Service, National Institutes of Health, Bethesda, Md. (1991)) and/or those residues from a “hypervariable loop” (e.g. residues 26-32 (L1), 50-52 (L2) and 91-96 (L3) in the VL, and 26-32 (H1), 53-55 (H2) and 96-101 (H3) in the VH; Chothia and Lesk J. Mol. Biol. 196:901-917 (1987)). There are multiple ways for determining CDRs, for example, an approach based on cross-species sequence variability (i.e., Kabat et al. Sequences of Proteins of Immunological Interest, (5th ed., 1991, National Institutes of Health, Bethesda Md.)); and an approach based on crystallographic studies of antigen-antibody complexes (Al-lazikani et al. (1997) J. Mol. Biol. 273:927-948)). The HVRs that are Kabat complementarity-determining regions (CDRs) are based on sequence variability and are the most commonly used (Kabat et al., supra). Chothia refers instead to the location of the structural loops (Chothia and Lesk J. Mol. Biol. 196:901-917 (1987)). The AbM HVRs represent a compromise between the Kabat CDRs and Chothia structural loops, and are used by Oxford Molecular's AbM antibody-modeling software. The “contact” HVRs are based on an analysis of the available complex crystal structures. As used herein, a CDR may be a CDR defined by any of the approaches or by a combination of any two or three of the approaches. The CDR may be Kabat CDR, Chothia CDR, or contact CDR. The residues from each of these HVRs are noted below.

| Loop | Kabat | AbM | Chothia | Contact |
|---|---|---|---|---|
| L1 | L24-L34 | L24-L34 | L26-L32 | L30-L36 |
| L2 | L50-L56 | L50-L56 | L50-L52 | L46-L55 |
| L3 | L89-L97 | L89-L97 | L91-L96 | L89-L96 |
| H1 | H31-H35B | H26-H35B | H26-H32 | H30-H35B |
| | | | | (Kabat numbering) |
| H1 | H31-H35 | H26-H35 | H26-H32 | H30-H35 |
| | | | | (Chothia numbering) |
| H2 | H50-H65 | H50-H58 | H53-H55 | H47-H58 |
| H3 | H95-H102 | H95-H102 | H96-H101 | H93-H101 |

HVRs may comprise “extended HVRs” as follows: 24-36 or 24-34 (L1), 46-56 or 50-56 (L2), and 89-97 or 89-96 (L3) in the VL, and 26-35 (H1), 50-65 or 49-65 (a preferred embodiment) (H2), and 93-102, 94-102, or 95-102 (H3) in the VH. The variable-domain residues are numbered according to Kabat et al., supra, for each of these extended-HVR definitions.

In some embodiments, the antibody is a cysteine engineered antibody comprising a free cysteine amino acid in the heavy chain or light chain (e.g., heavy chain and/or light chain constant region, and/or heavy chain and/or light chain variable region). Engineering of a free cysteine amino acid in the antibody may provide a reactive electrophilic functionality that may further enable antibody conjugate compounds such as antibody-drug conjugate (ADC) compounds with drug molecules at specific sites (i.e., site-specific conjugation). Examples of cysteine engineered antibodies and means to generate cysteine engineered antibodies are provided by Junutula, J R et al., (2008) Nat. Biotech. 26(8):925-932; Lyons, A et al., (1990) Prot. Engineering 3(8):703-708; and Stimmel, J B et al., (2000) J. Biol. Chem. 275(39):30445-30450. In some embodiments, the antibody is engineered to substitute amino acid residues (e.g., naturally occurring amino acids) on the heavy chain (e.g., in the constant region) or light chain (e.g., in the constant region) with one or more cysteine residues provided that the reactive thiol groups of the cysteine residues have little or no impact of antibody folding or assembly and do not significantly alter antigen binding. In some embodiments, the cysteine residues are evaluated for the reactivity of the newly introduced, engineered cysteine thiol groups. The thiol reactivity value is a relative, numerical term in the range of 0 to 1.0 and can be measured for any cysteine engineered antibody. In some embodiments, the thiol reactivity values of cysteine engineered antibodies of the invention are any one of about 0.6 to 1.0; 0.7 to 1.0; or 0.8 to 1.0. Cysteine engineered antibodies for site-specific conjugation of provided by WO 2006/034488, WO 2010/141902, WO 2013/093809, WO 2008/038024, WO 2008/070593, WO 2009/092011, WO 2011/005481 and WO 2011/156328.

A cysteine engineered antibody may be prepared by mutagenizing a nucleic acid sequence of a parent antibody by replacing one or more amino acid residues by cysteine to encode the cysteine engineered antibody; expressing the cysteine engineered antibody; and isolating the cysteine engineered antibody. In some embodiments, the cysteine engineered antibody is an antibody fragment; for example, a Fab, Fab′, F(ab′)2, Fv, or a single chain (ScFv) antibody. In some embodiments, the antibody is engineered to include one or more cysteine substitutions of amino acid residues 5157, T169 and S442 (EU numbering). In some embodiments of the invention, an antibody described herein (e.g., antibody hLL2, h10F4, g5/44, hHB22.7 or a humanized or chimeric antibody derived from antibody RFB4) or an antibody derived from any of these antibodies is engineered to comprise one or more free cysteine residues. The engineered cysteine residues are also referred to as “added cysteine residues”.

In some embodiments, one or more amino acid residues at any one or more of the following positions of the IgG heavy chain are replaced with a cysteine residue: 40, 43, 84, 88, 103, 112, 113, 114, 115, 131, 132, 133, 134, 135, 136, 137, 138, 139, 161, 168, 172, 234, 235, 237, 239, 246, 249, 265, 267, 269, 270, 276, 278, 282, 283, 284, 287, 289, 292, 293, 297, 298, 299, 300, 302, 303, 312, 314, 315, 318, 320, 324, 326, 327, 330, 332, 333, 334, 335, 336, 337, 339, 345, 347, 354, 355, 356, 358, 359, 360, 361, 362, 370, 373, 376, 378, 380, 382, 383, 384, 386, 388, 398, 390, 392, 393, 400, 401, 404, 411, 413, 414, 416, 418, 419, 421, 422, 428, 431, 432, 437, 438, 439, 440, 442, 443, and 444; numbering according to the EU index of Kabat et al. (1991, NIH Publication 91-3242, National Technical Information Service, Springfield, Va., hereinafter “Kabat”).

In some embodiments, one, two, three, four, five, six, seven, eight, nine, or ten or more amino acid residues at any combination of the following positions of the IgG heavy chain are replaced with a cysteine residue: 40, 43, 84, 88, 103, 112, 113, 114, 115, 131, 132, 133, 134, 135, 136, 137, 138, 139, 161, 168, 172, 234, 235, 237, 239, 246, 249, 265, 267, 269, 270, 276, 278, 282, 283, 284, 287, 289, 292, 293, 297, 298, 299, 300, 302, 303, 312, 314, 315, 318, 320, 324, 326, 327, 330, 332, 333, 334, 335, 336, 337, 339, 345, 347, 354, 355, 356, 358, 359, 360, 361, 362, 370, 373, 376, 378, 380, 382, 383, 384, 386, 388, 398, 390, 392, 393, 400, 401, 404, 411, 413, 414, 416, 418, 419, 421, 422, 428, 431, 432, 437, 438, 439, 440, 442, 443, and 444; numbering according to the EU index of Kabat.

In some embodiments, one or more amino acid residues at any one or more of the following positions of the IgG heavy chain (e.g., human IgG1, human IgG2, human IgG3, human IgG4, or human IgG4p) are replaced with a cysteine residue: T155, S157, S165, T169, T197, and S442, and/or V205 of the IgG light chain is replaced with a cysteine residue, wherein the numbering is according to the EU index of Kabat. The mutation position (EU numbering) and flanking sequences of amino acids are listed in Table 4 below.

TABLE 4
Mutation Position (EU Numbering) and Flanking
Sequences of Amino Acids

| EU | | SEQ ID |
|---|---|---|
| numbering | Flanking Sequence | NO: |
| V205C (CL) | EVTHQGLSSPCTKSFNRGEC | 57 |
| T155C | GCLVKDYFPEPVCVSWNSGALTSGV | 58 |
| (CH1) | (hIgG1~4) | |
| S157C (CH1) | LVKDYFPEPVTVCWNSGALTSGVHT | 59 |
| | (hIgG1~4) | |
| S165C (CH1) | PVTVSWNSGALTCGVHTFPAVLQSS | 60 |
| | (hIgG1~4) | |
| T169C (CH1) | SWNSGALTSGVHCFPAVLQSSGLYS | 61 |
| | (hIgG1~4) | |
| T197C (CH1) | VVTVPSSSLGTQCYICNVNHKPSNT (hIgG1) | 62 |
| | VVTVPSSNFGTQCYTCNVDHKPSNT (hIgG2) | 63 |
| | VVTVPSSSLGTQCYTCNVNHKPSNT (hIgG3) | 64 |
| | VVTVPSSSLGTKCYTCNVDHKPSNT (hIgG4) | 65 |
| S442C (CH3) | EALHNHYTQKSLCLSPGK (hIgG1, hIgG2) | 66 |
| | EALHNRFTQKSLCLSPGK (hIgG3) | 67 |
| | EALHNHYTQKSLCLSGK (hIgG4) | 68 |

In some embodiments, the anti-CD22 antibody described herein comprises a heavy constant region comprising an amino acid sequence set forth in SEQ ID NO: 12 or SEQ ID NO: 13. In some embodiments, the anti-CD22 antibody described herein comprises a light constant region comprising an amino acid sequence of SEQ ID NO: 11. In some embodiments, the anti-CD22 antibody described herein comprises a heavy constant region comprising an amino acid sequence of SEQ ID NO: 12 or SEQ ID NO: 13 and a light constant region comprising an amino acid sequence of SEQ ID NO: 11.

Additional positions on IgG heavy chain that can be engineered cysteine for site-specific conjugation include (EU numbering) 118-215, 234-239, 246, 248, 249, 254, 265, 267, 269, 270, 273, 276, 278, 279, 282, 283, 284, 286, 287, 289, 292, 293, 294, 297, 298, 299, 300, 302, 303, 312, 314, 315, 318, 320, 324, 326, 327, 330, 332-337, 339, 341-447 (described in US 2012/0148580 A1: WO 2013/093809 A1: US 2009/0258420 A1: U.S. Pat. No. 7,521,541 B2: U.S. Pat. No. 7,855,275 B2; US 2011/0137017 A1; US 2012/0213705 A1; US 2011/0033378 A1; U.S. Pat. No. 8,455,622 B2).

Additional positions on IgG light chain that can be engineered cysteine for site-specific conjugation include (EU numbering) 108-211 (described in WO 2013/093809 A1; US 2009/0258420 A1; U.S. Pat. No. 7,855,275 B2; U.S. Pat. No. 8,455,622 B2).

In some embodiments, one or more amino acid residues at any one or more of the following positions of the IgG lambda light chain are replaced with a cysteine residue: 7, 15, 20, 22, 25, 43, 110, 111, 125, 144, 149, 155, 158, 161, 168, 185, 188, 189, 191, 197, 205, 206, 207, 208 and 210, according to the EU index of Kabat.

In some embodiments, one, two, three, four, five, six, seven, eight, nine, or ten or more amino acid residues at any combination of the following positions of the IgG lambda light chain are replaced with a cysteine residue: 7, 15, 20, 22, 25, 43, 110, 111, 125, 144, 149, 155, 158, 161, 168, 185, 188, 189, 191, 197, 205, 206, 207, 208 and 210, according to the EU index of Kabat.

In some embodiments, one or more amino acid residues at any one or more of the following positions of the IgG kappa light chain are replaced with a cysteine residue: 7, 15, 20, 22, 25, 43, 110, 111, 144, 168, 183, and 210, according to the numbering of Kabat.

In some embodiments, one, two, three, four, five, six, seven, eight, nine, or ten or more amino acid residues at any combination of the following positions of the IgG kappa light chain are replaced with a cysteine residue: 7, 15, 20, 22, 25, 43, 110, 111, 144, 168, 183, and 210, according to the numbering of Kabat.

In some embodiments, the antibody is isolated. An isolated antibody refers to an antibody which has been identified and separated and/or recovered from a component of its natural environment. In some embodiments, the antibody is substantially pure. The term “substantially pure” may refer to material which is at least 50% pure (i.e., free from contaminants), more preferably at least 90% pure, more preferably at least 95% pure, more preferably at least 98% pure, more preferably at least 99% pure. In some embodiments, the antibody is a monoclonal antibody. In some embodiments, the antibody is a humanized antibody. In some embodiments, the antibody is a chimeric antibody. In some embodiments, the antibody is a human antibody. In some embodiments, the antibody is IgG (such as IgG₁, IgG₂, or IgG₄). In some embodiments, the antibody is human IgG such as human IgG₁. In some embodiments, the antibody is a human IgG comprising the IgG4p constant domain

The antibodies described herein may further include analogs and derivatives that are either modified, i.e., by the covalent attachment of any type of molecule as long as such covalent attachment permits the antibody to retain its antigen binding immunospecificity. For example, the derivatives and analogs of the antibodies include those that have been further modified, e.g., by glycosylation, acetylation, pegylation, phosphylation, amidation, derivatization by known protecting/blocking groups, proteolytic cleavage, linkage to a cellular ligand or other protein, etc. Chemical modifications can be carried out by known techniques, including, but not limited to specific chemical cleavage, acetylation, formulation, etc. Additionally, the analog or derivative can contain one or more unnatural amino acids.

In some embodiments, the antibody targeting moiety T in compounds of formulae (I)-(V), or a salt or solvate or stereoisomer thereof, is an antibody partially conjugated with a drug moiety, such that it may be further linked to additional drug moieties. Thus, in some embodiments, it is intended that a compound of the formula (I) or a salt or solvate or stereoisomer thereof embraces a compound of the formula (Ia) or a salt or solvate or stereoisomer thereof. Likewise, a compound of the formula (II) or a salt or solvate or stereoisomer thereof is intended to embrace a compound of the formula (IIa) or a salt or solvate or stereoisomer thereof; a compound of the formula (III) or a salt or solvate or stereoisomer thereof is intended to embrace a compound of the formula (IIIa) or a salt or solvate or stereoisomer thereof; a compound of the formula (IV) or a salt or solvate or stereoisomer thereof is intended to embrace a compound of the formula (IVa) or a salt or solvate or stereoisomer thereof; and a compound of the formula (V) or a salt or solvate or stereoisomer thereof is intended to embrace a compound of the formula (Va) or a salt or solvate or stereoisomer thereof.

Methods of making a targeting moiety (e.g., an antibody, a polypeptide, a peptide, or non-peptidyl moiety) are known in the art, such as the methods described in U.S. Pat. No. 7,674,605, U.S. Pat. No. 7,982,017, PCT/US2007/013587 (Publication No. WO 2007/146172), or PCT/US2008/087515 (Publication No. WO 2009/079649).

### Representative Linkers

In certain instances, the “-A-L⁴-L³-L²-” or “-A-L⁴-L³-” portion in the compound of Formula (I), (Ia), (II) or (IIa) is:

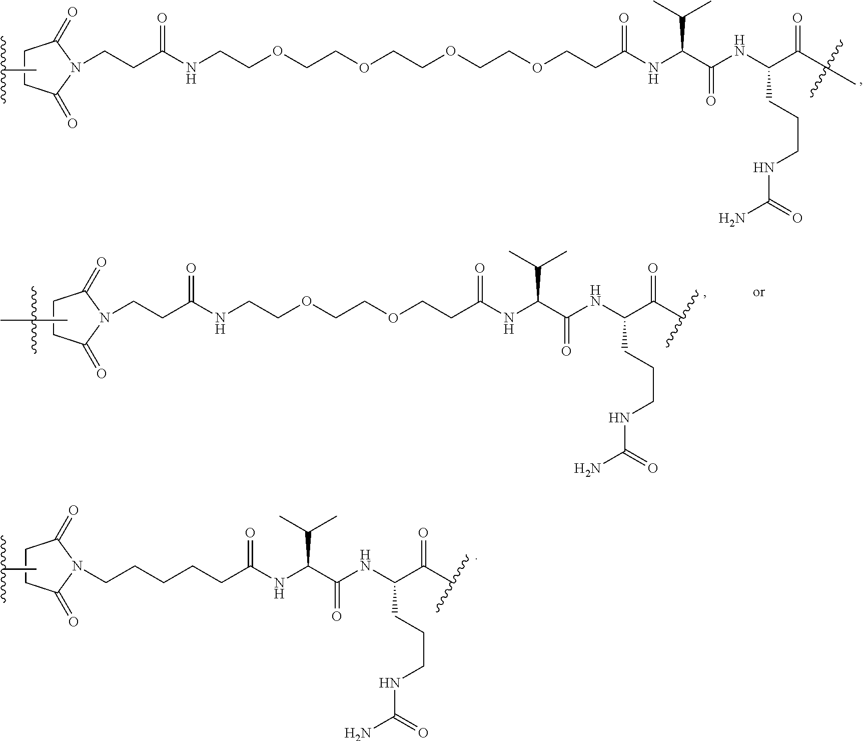

In certain instances, the “-A-L⁴-L³-L²-” or “-A-L⁴-L³-” portion in the compound of Formula (I), (Ia), (II) or (IIa) is:

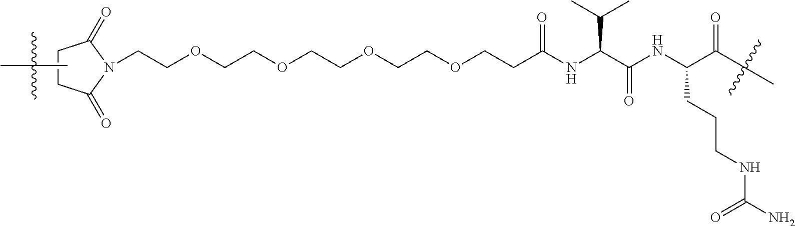

In certain instances, the “-A-L⁴-L³-L²-” or “-A-L⁴-L³-” portion in the compound of Formula (I), (Ia), (II) or (IIa) is:

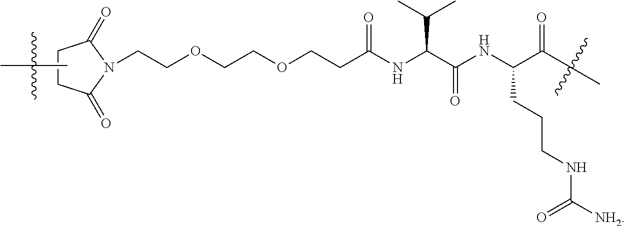

In certain instances, the “-A-L⁴-L³-L²-X-L¹-D” portion in the compound of Formula (I),

(Ia), (II) or (IIa) is:

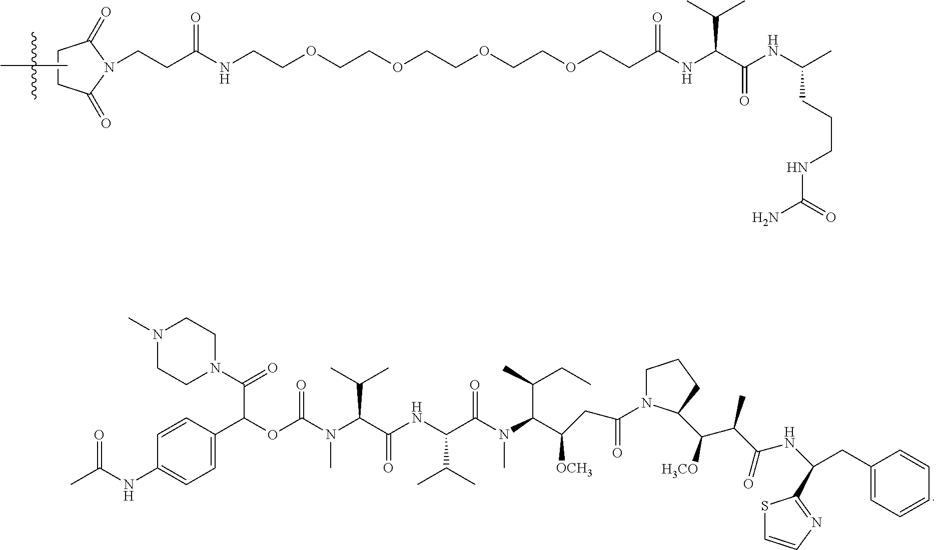

In such instance, the present disclosure provides a compound of Formula (III):

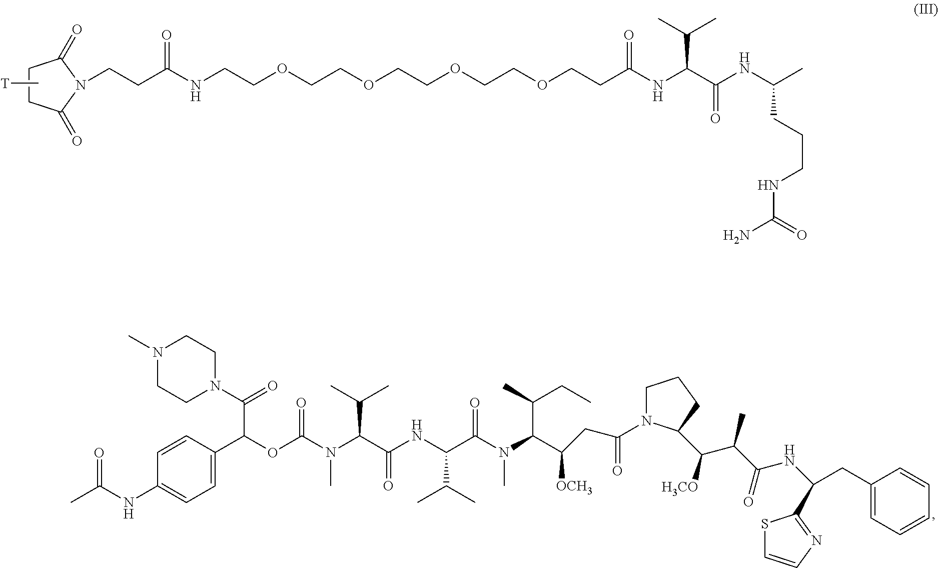

or a salt or solvate or stereoisomer thereof; wherein T is a targeting moiety. In certain instances, in Formula (III), T is an antibody. In some embodiments, T is an anti-CD22 antibody. In some embodiments, T is antibody hLL2, h10F4, g5/44, hHB22.7 or a derivative thereof. In some embodiments, T is a derivative of antibody RFB4 (e.g., such as a humanized or chimeric antibody). In some embodiments, provided is a compound of Formula (Ma):

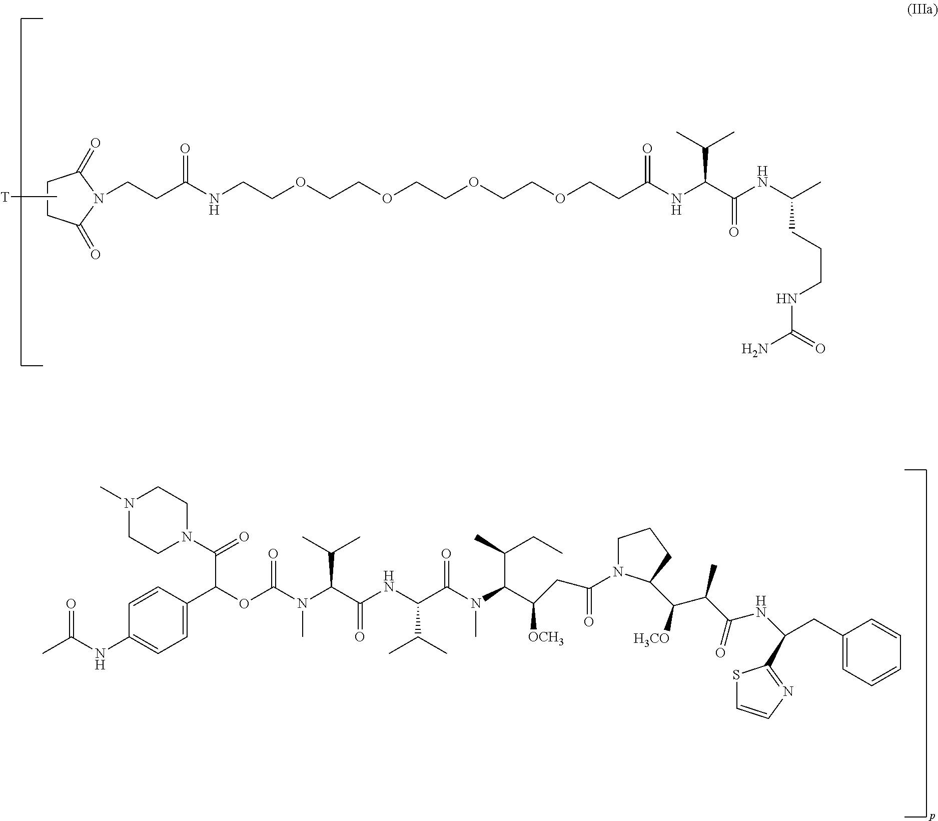

or a salt or solvate or stereoisomer thereof; wherein T is a targeting moiety and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3 or 4. In some embodiments, p is 2. In some embodiments, p is 3. In some embodiments, p is 4. In certain instances, in Formula (Ma), T is an antibody, optionally where one or more amino acid residues of the heavy chain and/or the light chain of the antibody are replaced with cysteine residues. In certain embodiments, the antibody is an anti-CD22 antibody. In some embodiments, the anti-CD22 antibody is hLL2, h10F4, g5/44, hHB22.7 or a derivative thereof, or hLL2 where one or more amino acid residues of the heavy chain and/or the light chain of the antibody are replaced with cysteine residues, or h10F4 where one or more amino acid residues of the heavy chain and/or the light chain of the antibody are replaced with cysteine residues, or g5/44 where one or more amino acid residues of the heavy chain and/or the light chain of the antibody are replaced with cysteine residues, or hHB22.7 where one or more amino acid residues of the heavy chain and/or the light chain of the antibody are replaced with cysteine residues. In some embodiments, the anti-CD22 antibody is a derivative of RFB4 (e.g., such a humanized or chimeric antibody), or a derivative of RFB4 (e.g., such as a humanized or chimeric antibody) where one or more amino acid residues of the heavy chain and/or the light chain of the antibody are replaced with cysteine residues.

In certain embodiments, a compound of formula (I) or (Ia), such as a compound of formula (III) or (Ma) can be prepared using synthetic intermediates such as a compound of formula (VI) or a salt or solvate thereof and/or a compound of formula (IX) or a salt or solvate thereof.

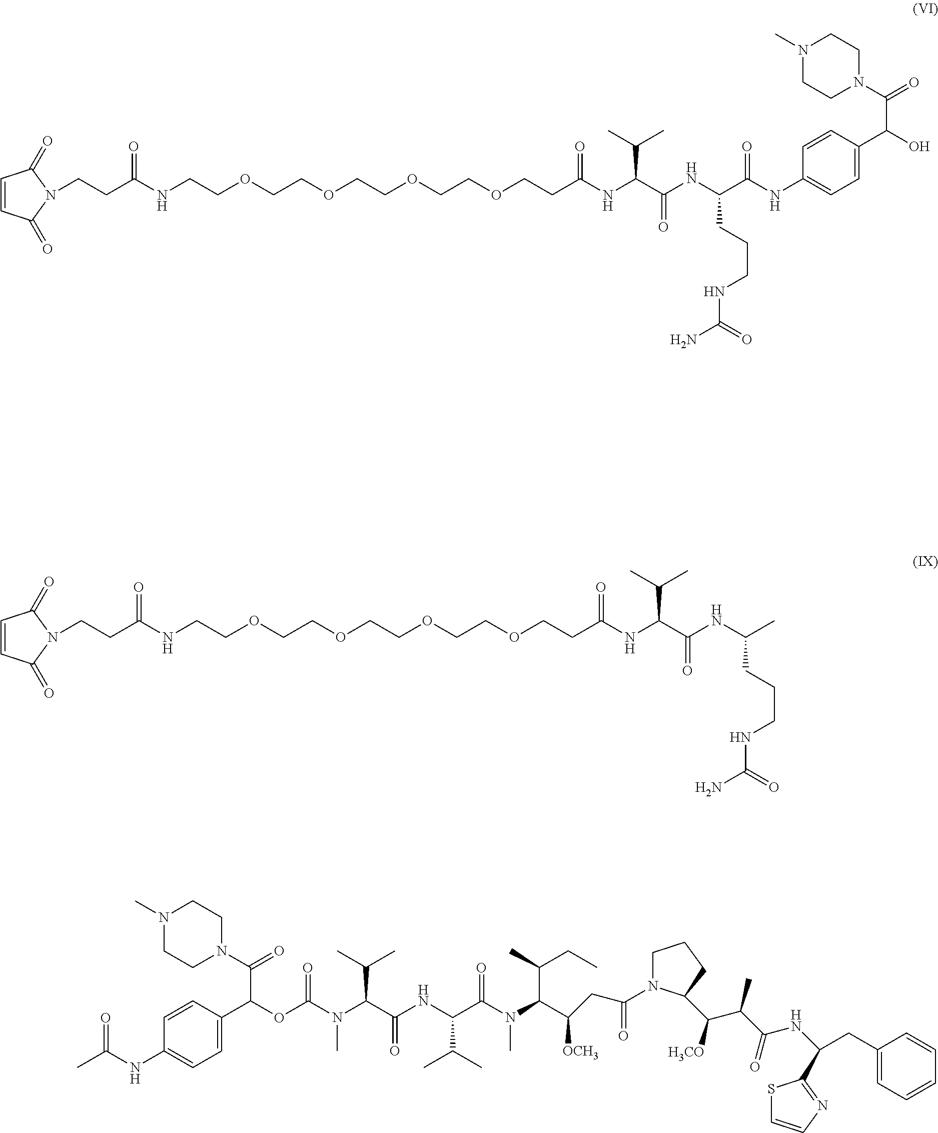 [SMILES: CC(C)[C@H](NC(=O)CCOCCOCCOCCOCCNC(=O)CCN1C(=O)C=CC1=O)C(=O)N[C@@H](CCCNC(N)=O)C(=O)Nc1ccc(C(O)C(=O)N2CCN(C)CC2)cc1 ; CC(C)[C@H](NC(=O)CCOCCOCCOCCOCCNC(=O)CCN1C(=O)C=CC1=O)C(=O)N[C@H](C)CCCNC(N)=O ; CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C(=O)OC(C(=O)N1CCN(C)CC1)c1ccc(NC(C)=O)cc1)C(C)C]

In certain instances, the “-A-L⁴-L³-L²-X-L¹-D” portion in the compound of Formula (I), (Ia), (II) or (IIa) is:

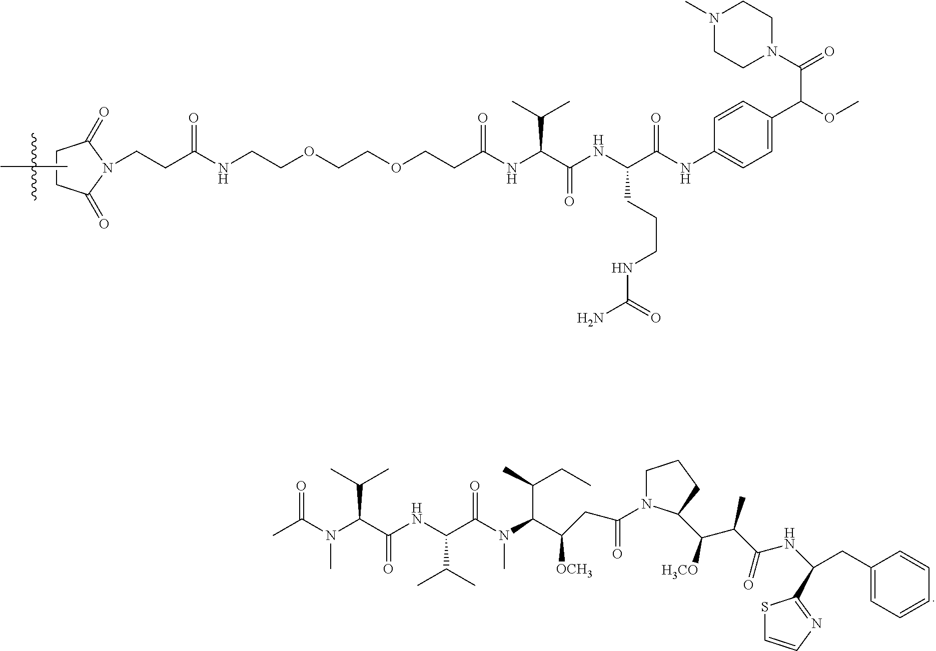

In such instance, the present disclosure provides a compound of Formula (IV):

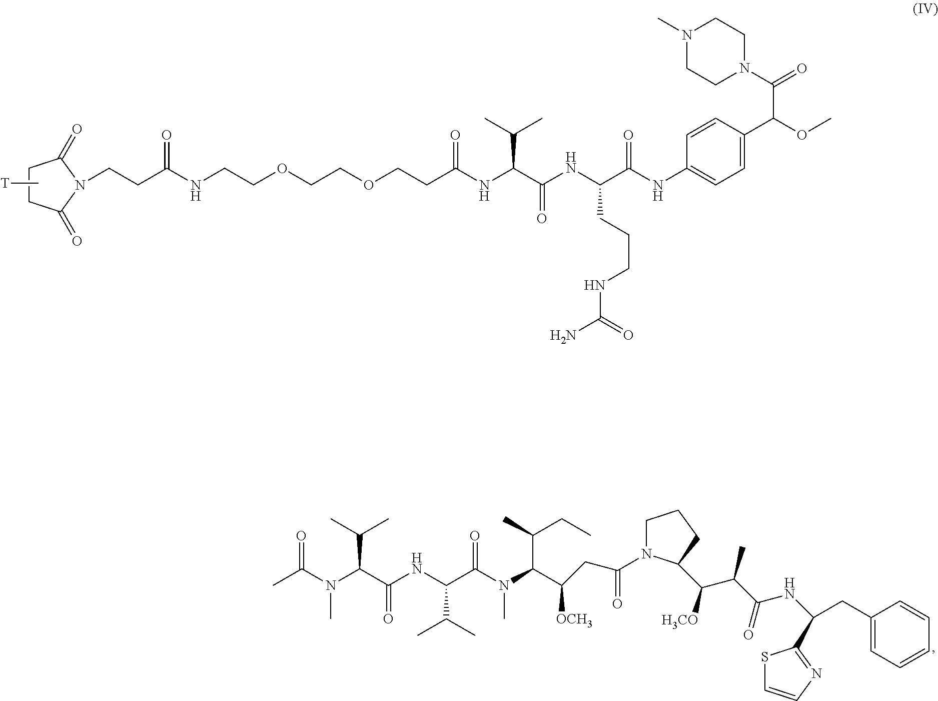

or a salt or solvate or stereoisomer thereof; wherein T is a targeting moiety. In certain instances, in Formula (IV), T is an antibody. In some embodiments, T is an anti-CD22 antibody. In some embodiments, the antibody is antibody hLL2, h10F4, g5/44, hHB22.7 or a derivative thereof. In some embodiments, the antibody is a derivative of antibody RFB4 (e.g., such as a humanized or chimeric antibody).

In some embodiments, provided is a compound of Formula (IVa):

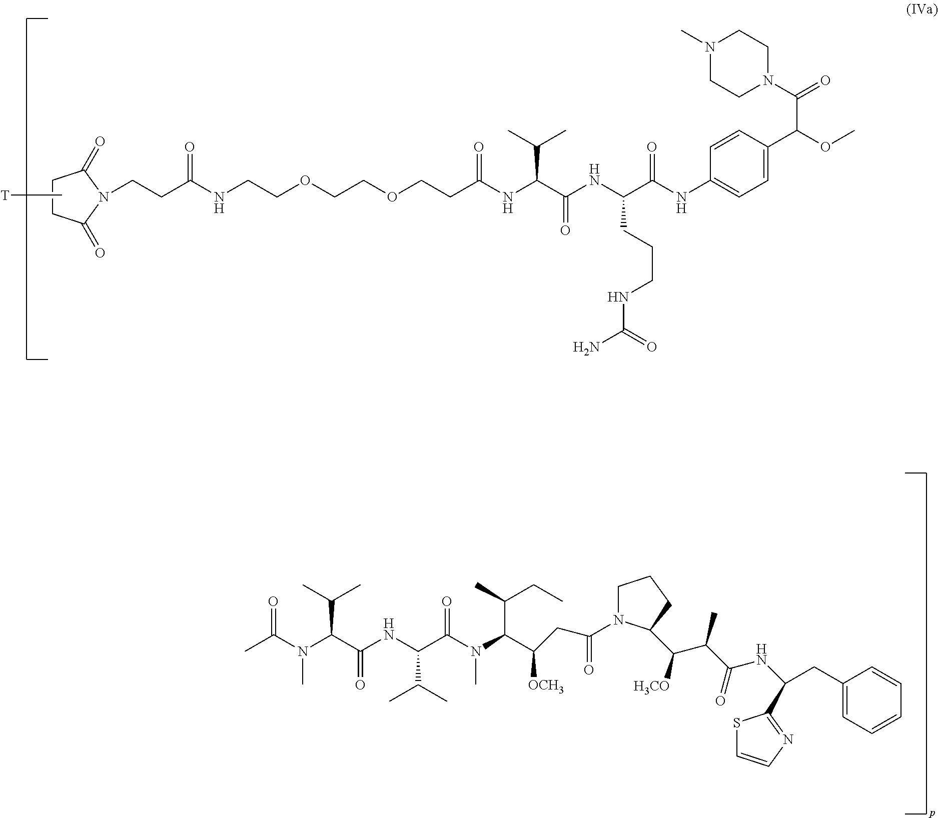

or a salt or solvate or stereoisomer thereof; wherein T is a targeting moiety and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3 or 4. In some embodiments, p is 2. In some embodiments, p is 3. In some embodiments, p is 4. In certain instances, in Formula (IVa), T is an antibody, optionally where one or more amino acid residues of the heavy chain and/or the light chain of the antibody are replaced with cysteine residues. In certain embodiments, the antibody is an anti-CD22 antibody. In some embodiments, the anti-CD22 antibody is hLL2, h10F4, g5/44, hHB22.7 or a derivative thereof, or hLL2 where one or more amino acid residues of the heavy chain and/or the light chain of the antibody are replaced with cysteine residues, or h10F4 where one or more amino acid residues of the heavy chain and/or the light chain of the antibody are replaced with cysteine residues, or g5/44 where one or more amino acid residues of the heavy chain and/or the light chain of the antibody are replaced with cysteine residues, or hHB22.7 where one or more amino acid residues of the heavy chain and/or the light chain of the antibody are replaced with cysteine residues. In some embodiments, the anti-CD22 antibody is a derivative of RFB4 (e.g., such a humanized or chimeric antibody), or a derivative of RFB4 (e.g., such as a humanized or chimeric antibody) where one or more amino acid residues of the heavy chain and/or the light chain of the antibody are replaced with cysteine residues.

In certain embodiments, a compound of formula (I) or (Ia), such as a compound of formula (IV) or (IVa) can be prepared using synthetic intermediates such as a compound of formula (VII) or a salt or solvate thereof and/or a compound of formula (X) or a salt or solvate thereof.

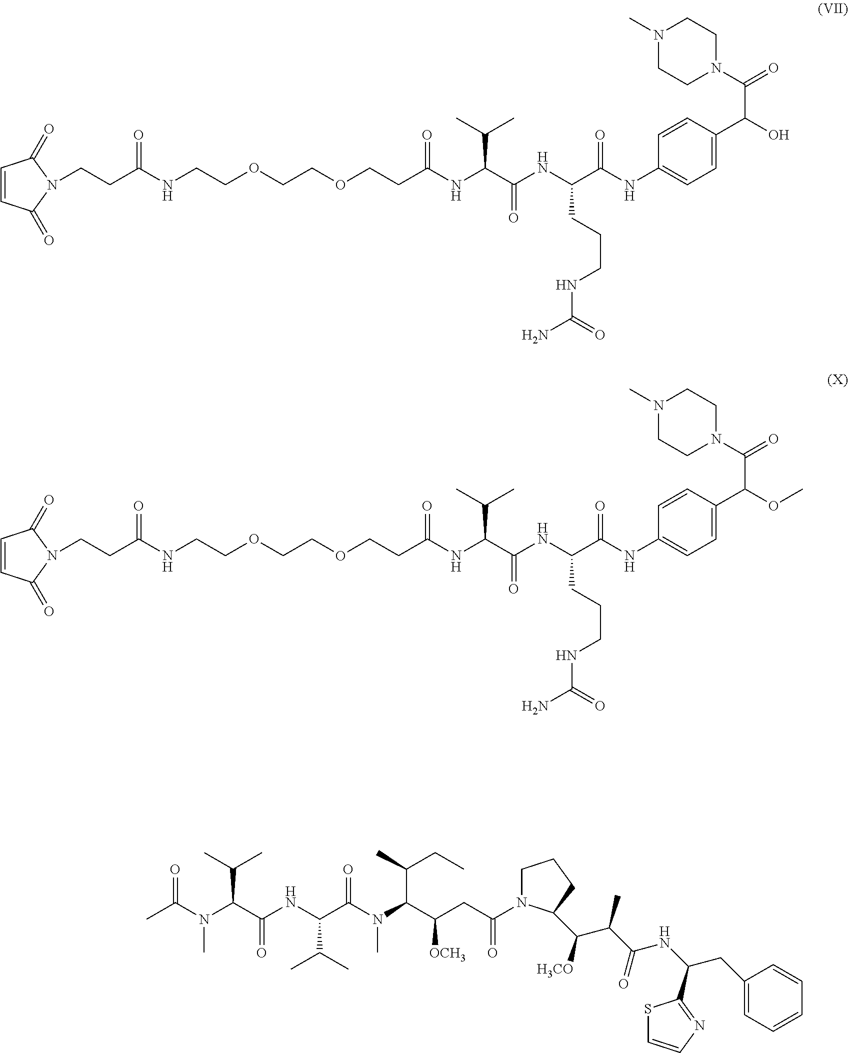

In certain instances, the “-A-L⁴-L³-L²-X-L¹-D” portion in the compound of Formula (I), (Ia), (II) or (IIa) is:

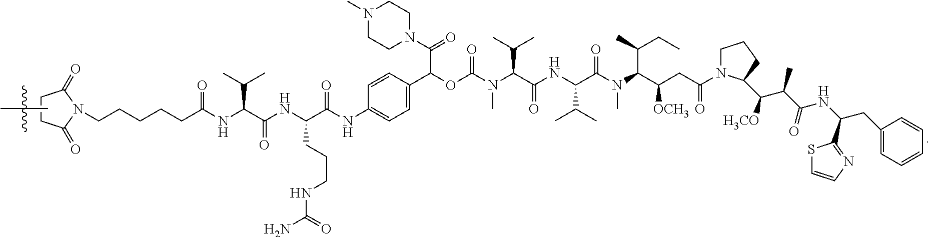

In such instance, the present disclosure provides a compound of Formula (V):

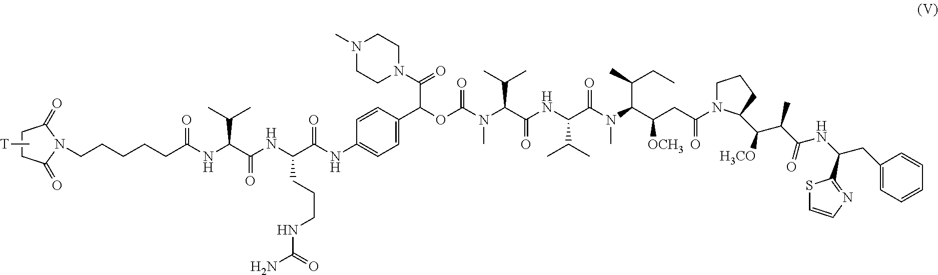

or a salt or solvate or stereoisomer thereof; wherein T is a targeting moiety. In certain instances, in Formula (V), T is an antibody. In some embodiments, the antibody is an anti-CD22 antibody. In some embodiments, the antibody is antibody hLL2, h10F4, g5/44, hHB22.7 or a derivative thereof. In some embodiments, the antibody is a derivative of antibody RFB4 (e.g., such as a humanized or chimeric antibody). In some embodiments, provided is a compound of Formula (Va):

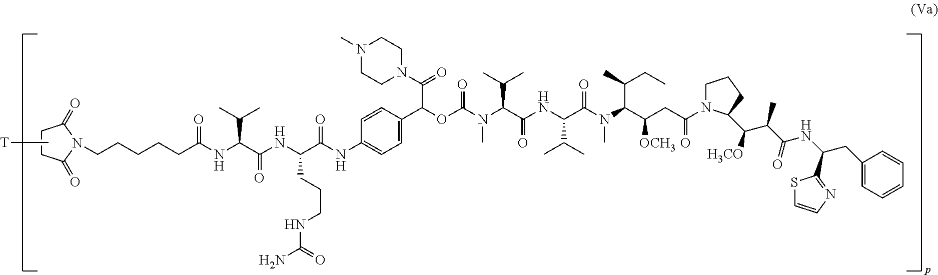

or a salt or solvate or stereoisomer thereof; wherein T is a targeting moiety and p is 1 to 20. In some embodiments, p is 1 to 8. In some embodiments, p is 1 to 6. In some embodiments, p is 1 to 4. In some embodiments, p is 2 to 4. In some embodiments, p is 1, 2, 3 or 4. In some embodiments, p is 2. In some embodiments, p is 3. In some embodiments, p is 4. In certain instances, in Formula (Va), T is an antibody, optionally where one or more amino acid residues of the heavy chain and/or the light chain of the antibody are replaced with cysteine residues. In certain embodiments, the antibody is an anti-CD22 antibody. In some embodiments, the antibody is antibody hLL2, h10F4, g5/44, hHB22.7 or a derivative thereof, or hLL2 where one or more amino acid residues of the heavy chain and/or the light chain of the antibody are replaced with cysteine residues, or h10F4 where one or more amino acid residues of the heavy chain and/or the light chain of the antibody are replaced with cysteine residues, or g5/44 where one or more amino acid residues of the heavy chain and/or the light chain of the antibody are replaced with cysteine residues, or hHB22.7 where one or more amino acid residues of the heavy chain and/or the light chain of the antibody are replaced with cysteine residues. In some embodiments, the antibody is a derivative of RFB4 (e.g., such a humanized or chimeric antibody), or a derivative of RFB4 (e.g., such as a humanized or chimeric antibody) where one or more amino acid residues of the heavy chain and/or the light chain of the antibody are replaced with cysteine residues.

In certain embodiments, a compound of formula (I) or (Ia), such as a compound of formula (V) or (Va) can be prepared using synthetic intermediates such as a compound of formula (VIII) or a salt or solvate thereof and/or a compound of formula (XI) or a salt or solvate thereof.

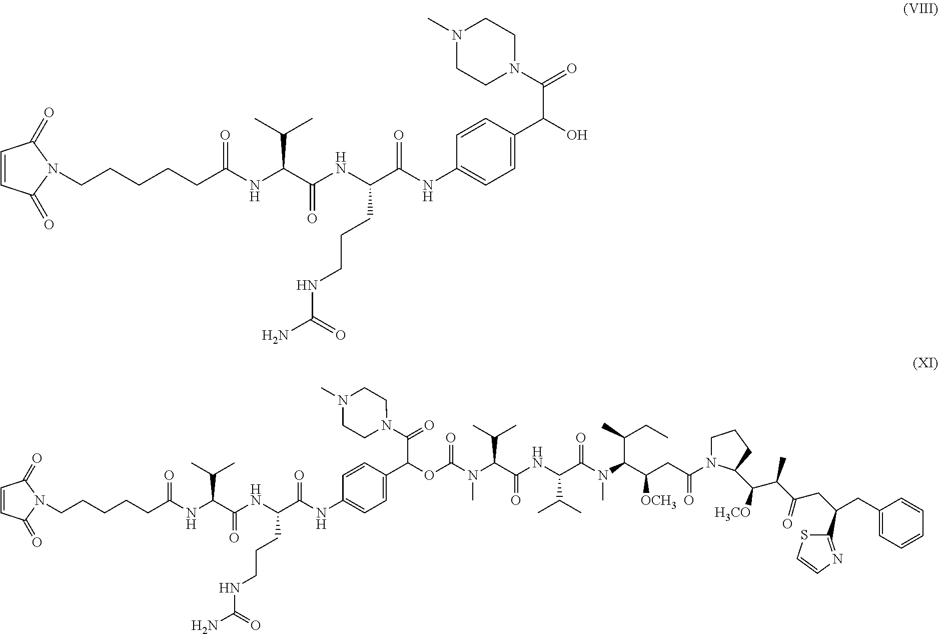 [SMILES: CC(C)[C@H](NC(=O)CCCCCN1C(=O)C=CC1=O)C(=O)N[C@@H](CCCNC(N)=O)C(=O)Nc1ccc(C(O)C(=O)N2CCN(C)CC2)cc1 ; CC[C@H](C)[C@@H]([C@@H](CC(=O)N1CCC[C@H]1[C@H](OC)[C@@H](C)C(=O)C[C@@H](Cc1ccccc1)c1nccs1)OC)N(C)C(=O)[C@@H](NC(=O)[C@H](C(C)C)N(C)C(=O)OC(C(=O)N1CCN(C)CC1)c1ccc(NC(=O)[C@H](CCCNC(N)=O)NC(=O)[C@@H](NC(=O)CCCCCN2C(=O)C=CC2=O)C(C)C)cc1)C(C)C]

In certain embodiments, a compound of formula (I) or (Ia), or any variations described herein, can be prepared using a compound of Formula (XII):

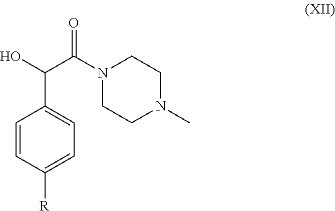

or a salt or solvate or stereoisomer thereof; wherein R is NO₂ or NH₂.

The compounds of Formulae (I)-(V) or (Ia)-(Va) may be prepared and/or formulated as pharmaceutically acceptable salts. Pharmaceutically acceptable salts are non-toxic salts of a free base form of a compound that possesses the desired pharmacological activity of the free base. These salts may be derived from inorganic or organic acids. Non-limiting examples of pharmaceutically acceptable salts include sulfates, pyrosulfates, bisulfates, sulfites, bisulfites, phosphates, monohydrogen-phosphates, dihydrogenphosphates, metaphosphates, pyrophosphates, chlorides, bromides, iodides, acetates, propionates, decanoates, caprylates, acrylates, formates, isobutyrates, caproates, heptanoates, propiolates, oxalates, malonates, succinates, suberates, sebacates, fumarates, maleates, butyne-1,4-dioates, hexyne-1,6-dioates, benzoates, chlorobenzoates, methylbenzoates, dinitrobenzoates, hydroxybenzoates, methoxybenzoates, phthalates, sulfonates, methylsulfonates, propylsulfonates, besylates, xylenesulfonates, naphthalene-1-sulfonates, naphthalene-2-sulfonates, phenylacetates, phenylpropionates, phenylbutyrates, citrates, lactates, γ-hydroxybutyrates, glycolates, tartrates, and mandelates. Lists of other suitable pharmaceutically acceptable salts are found in Remington's Pharmaceutical Sciences, 17th Edition, Mack Publishing Company, Easton, Pa., 1985.

For a compound of any one of Formulae (I)-(V) or (Ia)-(Va) that contains a basic nitrogen, a pharmaceutically acceptable salt may be prepared by any suitable method available in the art, for example, treatment of the free base with an inorganic acid, such as hydrochloric acid, hydrobromic acid, sulfuric acid, sulfamic acid, nitric acid, boric acid, phosphoric acid, and the like, or with an organic acid, such as acetic acid, phenylacetic acid, propionic acid, stearic acid, lactic acid, ascorbic acid, maleic acid, hydroxymaleic acid, isethionic acid, succinic acid, valeric acid, fumaric acid, malonic acid, pyruvic acid, oxalic acid, glycolic acid, salicylic acid, oleic acid, palmitic acid, lauric acid, a pyranosidyl acid, such as glucuronic acid or galacturonic acid, an alpha-hydroxy acid, such as mandelic acid, citric acid, or tartaric acid, an amino acid, such as aspartic acid or glutamic acid, an aromatic acid, such as benzoic acid, 2-acetoxybenzoic acid, naphthoic acid, or cinnamic acid, a sulfonic acid, such as laurylsulfonic acid, p-toluenesulfonic acid, methanesulfonic acid, or ethanesulfonic acid, or any compatible mixture of acids such as those given as examples herein, and any other acid and mixture thereof that are regarded as equivalents or acceptable substitutes in light of the ordinary level of skill in this technology.

Also provided are compositions comprising one or more compounds of the formulae (I)-(V) or (Ia)-(Va), or a salt or solvate or stereoisomers thereof. In the compounds of the formulae (I)-(V) or (Ia)-(Va), or a salt or solvate or stereoisomers thereof, the targeting moiety can have one or more sites of attachment for linking to the drug moiety. Depending on the accessibility of the attachment sites in the targeting moiety and the relative concentration of the drug moiety in forming the conjugate, a portion of the attachment sites may not be bonded to a drug moiety in the conjugate formed. A mixture of compounds having various number of drug moieties at each targeting moiety may form. Thus a composition is also provided, comprising one or more compounds of the formulae (Ia)-(Va), or a salt or solvate or stereoisomers thereof. For example, for a targeting molecule having 4 sites of attachment, the composition may comprise one or more compounds selected from a compound of formula (Ia) where p is 1, a compound of formula (Ia) where p is 2, a compound of formula (Ia) where p is 3, and a compound of formula (Ia) where p is 4. The relative amounts of compounds in the composition may be adjusted to achieve a desirable ratio between the drug moiety and the targeting moiety. In some of embodiments, the composition comprises predominantly one or two of the compounds.

The “drug-antibody ratio” (DAR) in a compound or composition of the invention is defined as the molar ratio between the drug moieties in the compound or composition and the antibodies in the compound or composition. Where an antibody has more than one site of attachment, more than one drug moiety may be linked to each antibody. In some instances, a mixture is obtained comprising more than one antibody-drug conjugate (ADC) molecules. The drug-antibody ratios of the antibody-drug conjugates can be measured by analytical methods know in the art, for example, methods as described in Jeffrey, et al., Bioconjug. Chem. 24(7):1256-1263 (2013); and Sun et al., Bioconjug. Chem. 16(5):1282-1290 (2005). In some embodiments, the composition comprising one or more ADCs of detailed herein has an average DAR of about 0.5 to about 6, about 1 to about 5, about 1 to about 4, about 1.5 to about 3.5, or about 2 to about 4. In some embodiments, the composition has an average DAR of about 1.5 to about 3.5, or about 2 to about 3.5, or about 2.7 to about 3.5, or about 2 to about 3, or about 3 to about 3.3, or about 2, or about 3. In some other preferred embodiments, the composition has an average DAR of about 2.5±10% (for example, about 2.25 to about 2.75). In some embodiments, the targeting antibody contains cysteine engineered sites of attachment and the composition has an average DAR of about 1.6 to about 2.1, or about 2.0.

### Pharmaceutical Compositions and Methods of Treatment

For treatment purposes, a pharmaceutical composition of the embodiments comprises at least one compound of Formulae (I)-(V) or (Ia)-(Va), or a pharmaceutically acceptable salt thereof. The pharmaceutical compositions may further comprise one or more pharmaceutically-acceptable excipients or pharmaceutically-acceptable carrier. A pharmaceutically-acceptable excipient is a substance that is non-toxic and otherwise biologically suitable for administration to a subject. Such excipients facilitate administration of the compounds described herein and are compatible with the active ingredient. Examples of pharmaceutically-acceptable excipients include stabilizers, lubricants, surfactants, diluents, anti-oxidants, binders, coloring agents, bulking agents, emulsifiers, or taste-modifying agents. In preferred embodiments, pharmaceutical compositions according to the embodiments are sterile compositions. Pharmaceutical compositions may be prepared using compounding techniques known or that become available to those skilled in the art.

Sterile compositions are also contemplated by the embodiments, including compositions that are in accord with national and local regulations governing such compositions.

The pharmaceutical compositions and compounds described herein may be formulated as solutions, emulsions, suspensions, dispersions, or inclusion complexes such as cyclodextrins in suitable pharmaceutical solvents or carriers, or as pills, tablets, lozenges, suppositories, sachets, dragees, granules, powders, powders for reconstitution, or capsules along with solid carriers according to conventional methods known in the art for preparation of various dosage forms. Pharmaceutical compositions of the embodiments may be administered by a suitable route of delivery, such as oral, parenteral, rectal, nasal, topical, or ocular routes, or by inhalation. Preferably, the compositions are formulated for intravenous or oral administration.

For oral administration, the compounds the embodiments may be provided in a solid form, such as a tablet or capsule, or as a solution, emulsion, or suspension. To prepare the oral compositions, the compounds of the embodiments may be formulated to yield a dosage of, e.g., from about 0.01 to about 50 mg/kg daily, or from about 0.05 to about 20 mg/kg daily, or from about 0.1 to about 10 mg/kg daily. Oral tablets may include the active ingredient(s) mixed with compatible pharmaceutically acceptable excipients such as diluents, disintegrating agents, binding agents, lubricating agents, sweetening agents, flavoring agents, coloring agents and preservative agents. Suitable inert fillers include sodium and calcium carbonate, sodium and calcium phosphate, lactose, starch, sugar, glucose, methyl cellulose, magnesium stearate, mannitol, sorbitol, and the like. Exemplary liquid oral excipients include ethanol, glycerol, water, and the like. Starch, polyvinyl-pyrrolidone (PVP), sodium starch glycolate, microcrystalline cellulose, and alginic acid are exemplary disintegrating agents. Binding agents may include starch and gelatin. The lubricating agent, if present, may be magnesium stearate, stearic acid, or talc. If desired, the tablets may be coated with a material such as glyceryl monostearate or glyceryl distearate to delay absorption in the gastrointestinal tract, or may be coated with an enteric coating.

Capsules for oral administration include hard and soft gelatin capsules. To prepare hard gelatin capsules, active ingredient(s) may be mixed with a solid, semi-solid, or liquid diluent. Soft gelatin capsules may be prepared by mixing the active ingredient with water, an oil such as peanut oil or olive oil, liquid paraffin, a mixture of mono and di-glycerides of short chain fatty acids, polyethylene glycol 400, or propylene glycol.

Liquids for oral administration may be in the form of suspensions, solutions, emulsions, or syrups, or may be lyophilized or presented as a dry product for reconstitution with water or other suitable vehicle before use. Such liquid compositions may optionally contain: pharmaceutically-acceptable excipients such as suspending agents (for example, sorbitol, methyl cellulose, sodium alginate, gelatin, hydroxyethylcellulose, carboxymethylcellulose, aluminum stearate gel and the like); non-aqueous vehicles, e.g., oil (for example, almond oil or fractionated coconut oil), propylene glycol, ethyl alcohol, or water; preservatives (for example, methyl or propyl p-hydroxybenzoate or sorbic acid); wetting agents such as lecithin; and, if desired, flavoring or coloring agents.

The compositions of the embodiments may be formulated for rectal administration as a suppository. For parenteral use, including intravenous, intramuscular, intraperitoneal, intranasal, or subcutaneous routes, the agents of the embodiments may be provided in sterile aqueous solutions or suspensions, buffered to an appropriate pH and isotonicity or in parenterally acceptable oil. Suitable aqueous vehicles include Ringer's solution and isotonic sodium chloride. Such forms may be presented in unit-dose form such as ampoules or disposable injection devices, in multi-dose forms such as vials from which the appropriate dose may be withdrawn, or in a solid form or pre-concentrate that can be used to prepare an injectable formulation. Illustrative infusion doses range from about 1 to 1000 μg/kg/minute of agent admixed with a pharmaceutical carrier over a period ranging from several minutes to several days.

For nasal, inhaled, or oral administration, the pharmaceutical compositions of the embodiments may be administered using, for example, a spray formulation also containing a suitable carrier.

For topical applications, the compounds of the embodiments are preferably formulated as creams or ointments or a similar vehicle suitable for topical administration. For topical administration, the inventive compounds may be mixed with a pharmaceutical carrier at a concentration of about 0.1% to about 10% of drug to vehicle. Another mode of administering the agents of the embodiments may utilize a patch formulation to effect transdermal delivery.

The present disclosure provides a method of killing a cell that expresses a CD22, comprising administering to the cell an effective amount of the compound of Formulae (I)-(V) or (Ia)-(Va) or a salt, a solvate, or a stereoisomer thereof, sufficient to kill the cell. In certain embodiments, the cell is a cancer cell. In certain embodiments, the cancer cell is a CD22-positive hematological malignant cell (e.g., a lymphoma or a leukemia).

In another aspect, the present disclosure provides a method of treating cancer in an individual comprising administering to the individual an effective amount of a compound of Formulae (I)-(V) or (Ia)-(Va) or a salt, a solvate, or a stereoisomer thereof. Examples of cancers that may be treated with the method described herein include, but are not limited to, carcinoma, including adenocarcinoma, lymphoma, blastoma, melanoma, and sarcoma. More particular examples of such cancers include squamous cell cancer, small-cell lung cancer, non-small cell lung cancer, lung adenocarcinoma, lung squamous cell carcinoma, gastrointestinal cancer, Hodgkin's and non-Hodgkin's lymphoma, pancreatic cancer, glioblastoma, cervical cancer, glioma, ovarian cancer, liver cancer such as hepatic carcinoma and hepatoma, bladder cancer, breast cancer, colon cancer, colorectal cancer, endometrial or uterine carcinoma, salivary gland carcinoma, kidney cancer such as renal cell carcinoma and Wilms' tumors, basal cell carcinoma, melanoma, mesothelioma, prostate cancer, thyroid cancer, testicular cancer, esophageal cancer, gallbladder cancer, and various types of head and neck cancer.

In certain embodiments of the method for treating cancer, the cancer is a CD22-positive hematological malignancy. In certain embodiments, the CD22-positive hematological malignancy is B-cell lymphoma or acute lymphoblastic leukemia. In certain embodiments, the individual has cancer or has been diagnosed with cancer. In certain embodiments, the individual has a CD22-positive hematological malignancy or has been diagnosed with a CD22-positive hematological malignancy. In certain embodiments, the individual is a human. In some embodiments, the method further comprises a step of detecting the expression level of CD22 on cancer cells before administering the compound. In some embodiments, the compound is administered intraperitoneally, intravenously, intramuscularly, subcutaneously, intrathecally, intraventricularly, orally, enterally, parenterally, intranasally, dermally, sublingually, or by inhalation.

### Kits

The present disclosure provides a pharmaceutical pack or kit comprising one or more containers comprising a compound of Formulae (I)-(V) or (Ia)-(Va), or a salt, solvate or stereoisomer thereof, useful for the treatment or prevention of cancer. The kit can further comprise instructions for use in the treatment of cancer.

The present disclosure also provides a pharmaceutical pack or kit comprising one or more containers comprising one or more of the ingredients of the pharmaceutical compositions of the present embodiments. Optionally associated with such container(s) can be a notice in the form prescribed by a governmental agency regulating the manufacture, use or sale of pharmaceuticals or biological products, which notice reflects approval by the agency of manufacture, use or sale for human administration.

### Synthesis of Drug Conjugates

The embodiments are also directed to processes and intermediates useful for preparing subject compounds or a salt or solvate or stereoisomer thereof.

Many general references providing commonly known chemical synthetic schemes and conditions useful for synthesizing the disclosed compounds are available (see, e.g., Smith and March, March's Advanced Organic Chemistry: Reactions, Mechanisms, and Structure, Fifth Edition, Wiley-Interscience, 2001.)

Compounds as described herein can be purified by any of the means known in the art, including chromatographic means, such as high performance liquid chromatography (HPLC), preparative thin layer chromatography, flash column chromatography and ion exchange chromatography. Any suitable stationary phase can be used, including normal and reversed phases as well as ionic resins. Most typically the disclosed compounds are purified via silica gel and/or alumina chromatography. See, e.g., Introduction to Modem Liquid Chromatography, 2nd ed., ed. L. R. Snyder and J. J. Kirkland, John Wiley and Sons, 1979; and Thin Layer Chromatography, E. Stahl (ed.), Springer-Verlag, New York, 1969.

During any of the processes for preparation of the subject compounds, it may be necessary and/or desirable to protect sensitive or reactive groups on any of the molecules concerned. This may be achieved by means of conventional protecting groups as described in standard works, such as T. W. Greene and P. G. M. Wuts, “Protective Groups in Organic Synthesis,” 4^{th} ed., Wiley, New York 2006. The protecting groups may be removed at a convenient subsequent stage using methods known from the art.

Exemplary chemical entities useful in methods of the embodiments will now be described by reference to illustrative synthetic schemes for their general preparation herein and the specific examples that follow. Artisans will recognize that, to obtain the various compounds herein, starting materials may be suitably selected so that the ultimately desired substituents will be carried through the reaction scheme with or without protection as appropriate to yield the desired product. Alternatively, it may be necessary or desirable to employ, in the place of the ultimately desired substituent, a suitable group that may be carried through the reaction scheme and replaced as appropriate with the desired substituent. Furthermore, one of skill in the art will recognize that the transformations shown in the schemes below may be performed in any order that is compatible with the functionality of the particular pendant groups. Each of the reactions depicted in the general schemes is preferably run at a temperature from about 0° C. to the reflux temperature of the organic solvent used. Unless otherwise specified, the variables are as defined above in reference to Formula (I).

The conjugates of the present embodiments may be constructed by attaching the drug moiety to the antibody through a linker comprising a hydrophilic self-immolative spacer.

Representative syntheses for the linker portion of compounds of Formula (I) are described in schemes below, and the particular examples that follow.

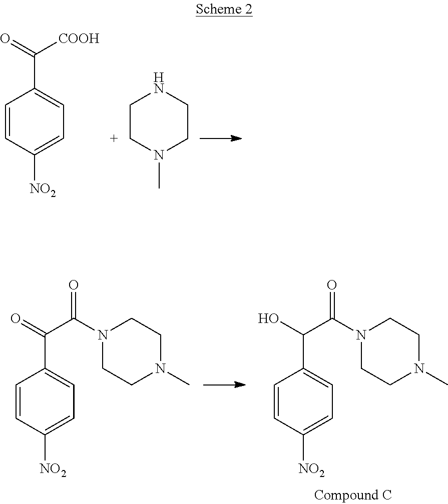

Synthesis of Compound C from 4-nitrobenzaldehyde is shown above in Scheme 2. 4-Nitrophenylglycolic acid is converted to the corresponding acid chloride using a chlorinating reagent, such as SOCl₂, PCl₃, or PCl₅. The acid chloride is then reacted with 1-methylpiperazine to give the ketoamide intermediate. Alternatively, the 4-nitrophenylglycolic acid can be coupled to the 1-methylpiperazine with use of coupling agent, such as EDCI. The ketoamide intermediate contains a keto group, which is then reduced with a reducing reagent, such as DIBAL-H, BH₃, LiAlH₄—AlCl₃, LiA1H₄—BF₃-Et₂O, or sodium borohydride, to produce Compound C.

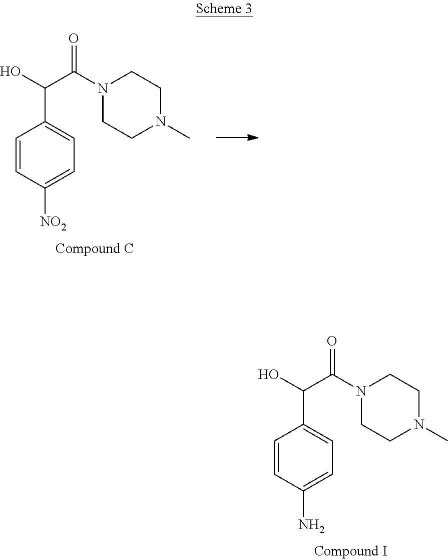 [SMILES: CN1CCN(C(=O)C(O)c2ccc([N+](=O)[O-])cc2)CC1 ; CN1CCN(C(=O)C(O)c2ccc(N)cc2)CC1]

Referring to Scheme 3, the nitro group of Compound C is reduced to yield an aniline group in Compound I by catalytic hydrogenation with catalysts, such as palladium, nickel, or platinum. Examples of suitable hydrogenation catalysts include Pd/C and Raney nickel.

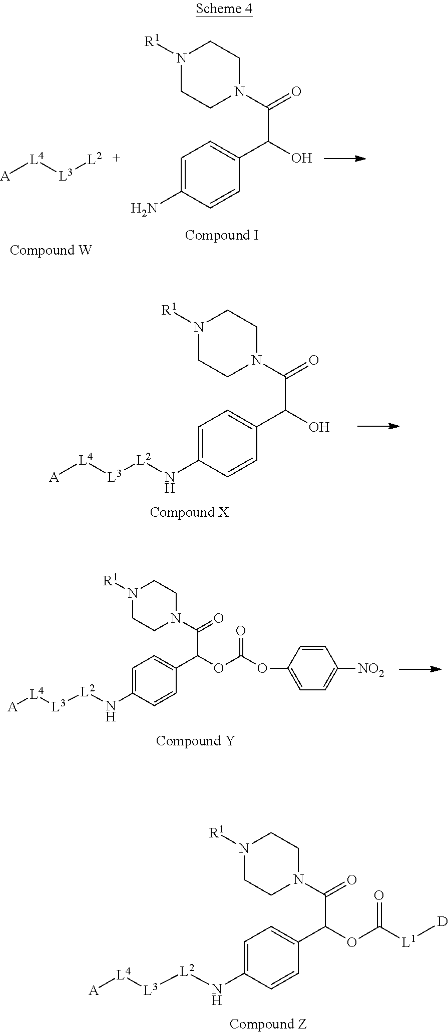

Referring to Scheme 4, Compound I provides the hydrophilic self-immolative linker portion in the compounds of the present embodiments. The amino group of Compound I can react with the Compound W through standard peptide coupling conditions to produce Compound X. Reagents such as EDCl/HOBt, HOBt, PyBOP, HATU or BEM (Carpino, L. A. J. Am. Chem. Soc. 1993, 115, 4397. Carpino, L. A.; El-Faham, A. J. Am. Chem. Soc. 1995, 117, 5401. Li, P.; Xu, J. C. J. Pept. Res. 2001, 58, 129.) in the presence of a base such as DIEA or other bases familiar to one skilled in the art and in an appropriate solvent can be used.

With continued reference to Scheme 4, the hydroxyl group of Compound X is converted to an activated carbonate using 4-nitrophenyl chloroformate. With Compound Y, reaction with a drug with an amino group can produce Compound Z. If the drug does not contain an amino group, a second, intermediate self-immolative spacer or a cyclization self-elimination linker can be situated between the drug moiety and the aminobenzyloxycarbonyl group, as discussed above.

In certain embodiments, referring to Scheme 5 below, the -L³-L²-portion of the linker is attached to Compound I. Then the -A-L⁴-portion is attached.

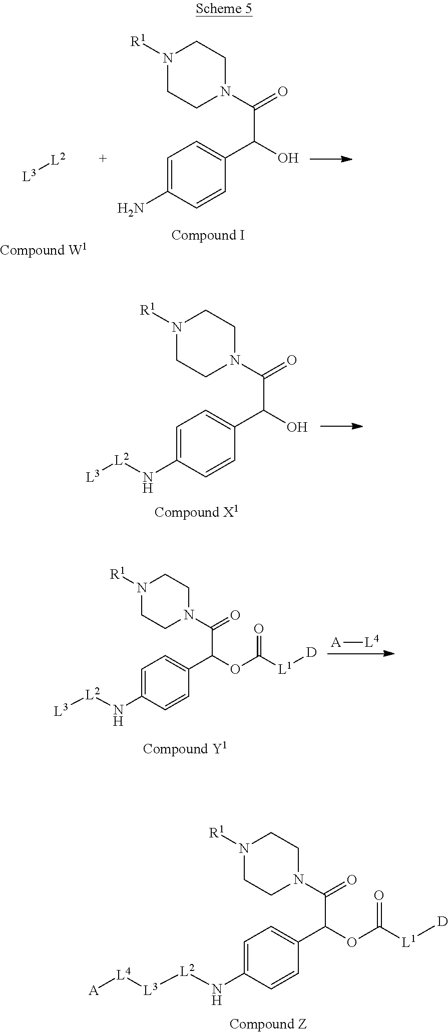

A process for preparing the compound of the present embodiments includes preparing a solution of the antibody in a buffer and treating with a solution of reducing agent, such as TCEP. The amount of free thiols is determined. When the amount of free thiols reaches a predetermined amount, the partially reduced antibody is alkylated with the linker-drug portion.

In some embodiments, provided is a process for making a compound of formula (I) or (Ia):

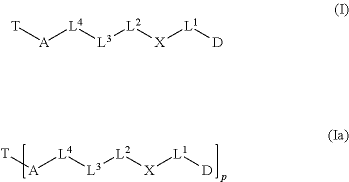

or a salt or solvate or stereoisomer thereof; wherein D, T, X, L¹, L², L³, L⁴, A and p, where applicable, are as defined for Formula (I) or (Ia), comprising reacting a compound comprising a targeting moiety T with a compound of formula: A-L⁴-L³-L²-X-L¹-D. In some embodiments, T is an antibody that specifically binds to a CD22 (e.g. a human CD22). In some embodiments, provided is a compound produced by the process. Further provided is a composition comprising one or more compounds produced by the process.

In some embodiments, provided is a process for making a compound of formula (II) or (IIa):

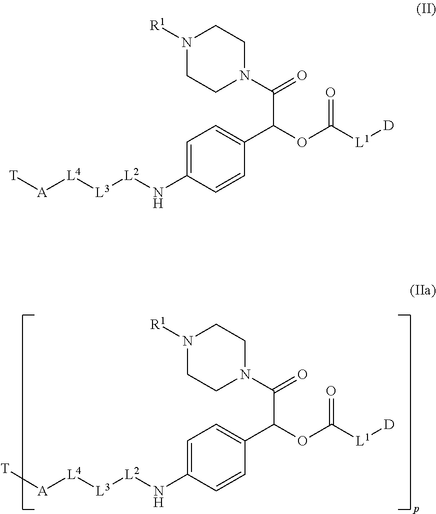

or a salt or solvate or stereoisomer thereof; wherein D, T, L¹, L², L³, L⁴, A and p, where applicable, are as defined for Formula (II) or (IIa), comprising reacting an antibody bearing one or more free thiols (or sulfhydryl groups) with Compound Z:

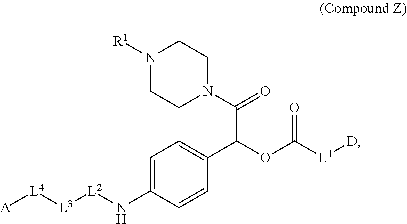

or a salt or solvate or stereoisomer thereof. In certain embodiments, the antibody bearing one or more free thiols (or sulfhydryl groups) is an anti-CD22 antibody. In some embodiments, antibody bearing one or more free thiols (or sulfhydryl groups) is hLL2, h10F4, g5/44, hHB22.7 or a derivative thereof, or hLL2 where one or more amino acid residues of the heavy chain and/or the light chain of the antibody are replaced with cysteine residues, or h10F4 where one or more amino acid residues of the heavy chain and/or the light chain of the antibody are replaced with cysteine residues, or g5/44 where one or more amino acid residues of the heavy chain and/or the light chain of the antibody are replaced with cysteine residues, or hHB22.7 where one or more amino acid residues of the heavy chain and/or the light chain of the antibody are replaced with cysteine residues. In some embodiments, antibody bearing one or more free thiols (or sulfhydryl groups) is a derivative of RFB4 (e.g., such a humanized or chimeric antibody), or a derivative of RFB4 (e.g., such as a humanized or chimeric antibody) where one or more amino acid residues of the heavy chain and/or the light chain of the antibody are replaced with cysteine residues. In some embodiments, the process further comprises a method for preparing Compound Z as detailed herein. In some embodiments, the process further comprises a method for preparing one or more of the synthetic intermediates leading to Compound Z (e.g., Compound Y and Compound X) as detailed herein. In some embodiments, provided is a compound produced by any of the processes detailed herein. Further provided is a composition comprising one or more compounds produced by any of the processes detailed herein.

In some embodiments, a process is provided for making a compound of formula (II):

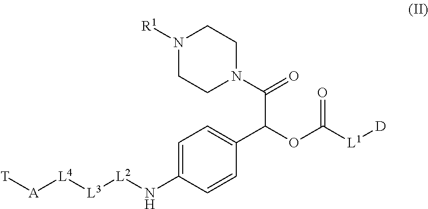

or a salt or solvate or stereoisomer thereof;

wherein:

D is a drug moiety;

T is an antibody;

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit;

comprising reacting an antibody with Compound Z:

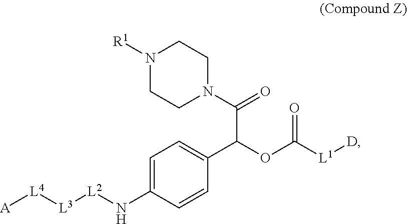

or a salt or solvate or stereoisomer thereof. In some embodiments, T is an antibody that specifically binds to a CD22 (e.g., a human CD22).

In some embodiments, a process is provided for making a compound of formula (IIa):

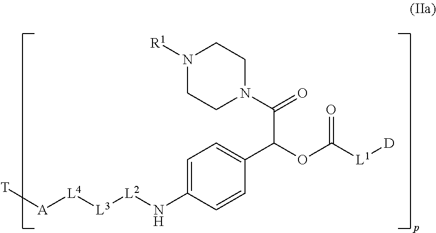

or a salt or solvate or stereoisomer thereof;

wherein:

p is 1 to 20;

D is a drug moiety;

T is an antibody;

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit;

comprising reacting an antibody with Compound Z:

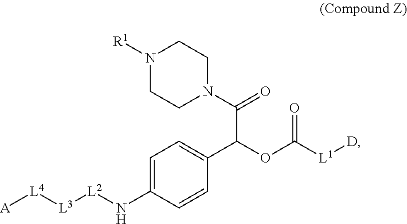

or a salt or solvate or stereoisomer thereof. In some embodiments, T is an antibody that specifically binds to a CD22 (e.g., a human CD22).

Further provided is a compound produced by any of the processes of making compounds and/or methods of preparing compounds as detailed herein. Also provided is a composition (e.g., a pharmaceutical composition) comprising one or more of the compounds produced by any of the processes of making compounds and/or methods of preparing compounds as detailed herein.

The present disclosure provides for the process for the preparation of the compounds and intermediates in Schemes 4 and 5. The compounds represented in Schemes 4 and 5 are meant to have full valences or properly capped with optional protecting groups or leaving groups when appropriate. For example, as shown in the scheme “Synthesis of Compound TAP-18H,” L³-L² can be

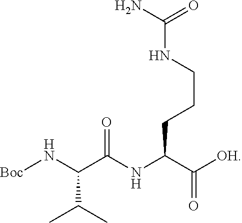 [SMILES: CC(C)[C@H](NC(=O)OC(C)(C)C)C(=O)N[C@@H](CCCNC(N)=O)C(=O)O]

The present disclosure provides for a method of preparing Compound X:

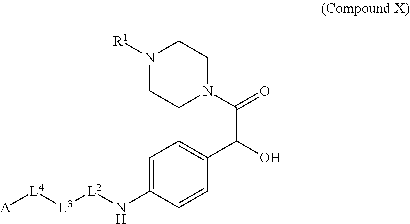

or a salt or solvate or stereoisomer thereof;

wherein:

R¹ is hydrogen, unsubstituted or substituted C₁₋₃ alkyl, or unsubstituted or substituted heterocyclyl;

L² is a bond or a second self-immolative linker;

L³ is a peptide linker;

L⁴ is a bond or a spacer; and

A is an acyl unit;

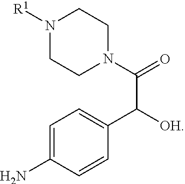

comprising: reacting Compound W: A-L⁴-L³-L², and Compound I:

The present disclosure provides for a method of preparing Compound Z:

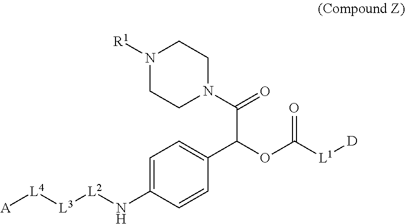

or a salt or solvate or stereoisomer thereof;

wherein:

D is a drug moiety;

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit;

comprising: reacting Compound X:

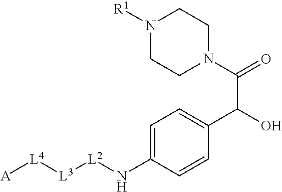

with a compound comprising L¹-D.

In some embodiments, the method of preparing Compound Z comprises reacting Compound X in the presence of a carboxylic acid activating agent to form Compound Y^{a}:

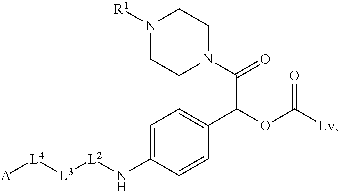

where Lv is a leaving group; and

reacting Compound Y^{a} with a compound comprising L¹-D. In some embodiments, the carboxylic acid activating agent is p-nitrophenyl chloroformate, and Lv is p-nitrophenyl. Other carboxylic acid activating agents known in the art can be used in place of p-nitrophenyl chloroformate, such as bis(4/p-nitrophenyl) carbonate, phosgene, triphosgene/bis(trichloromethyl carbonate), trichloromethyl chloroformate, N,N′-disuccinimidyl carbonate, and 1,1′-carbonyldiimidazole.

In some embodiments, the method of preparing Compound Z comprises reacting Compound X and p-nitrophenyl chloroformate to form Compound Y:

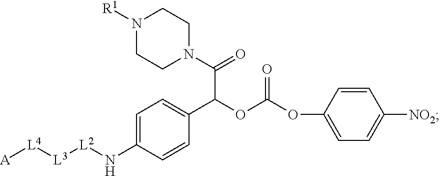

and

reacting Compound Y with a compound comprising L¹-D.

The present disclosure provides for a method of preparing Compound X¹:

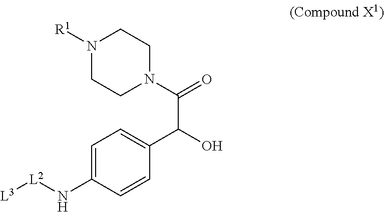

or a salt or solvate or stereoisomer thereof;

wherein:

R¹ is hydrogen, unsubstituted or substituted C₁₋₃ alkyl, or unsubstituted or substituted heterocyclyl;

L² is a bond or a second self-immolative linker; and

L³ is a peptide linker;

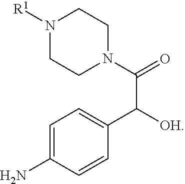

comprising: reacting Compound W¹: L³-L², and Compound I:

The present disclosure provides for a method of preparing Compound Y¹:

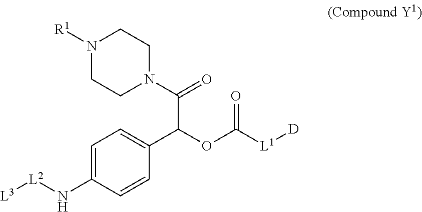

or a salt or solvate or stereoisomer thereof;

wherein:

R¹ is hydrogen, unsubstituted or substituted C₁₋₃ alkyl, or unsubstituted or substituted heterocyclyl;

D is a drug moiety;

L¹ is a bond, a second self-immolative linker, or a cyclization self-elimination linker;

L² is a bond or a second self-immolative linker;

wherein if L¹ is a second self-immolative linker or a cyclization self-elimination linker, then L² is a bond;

wherein if L² is a second self-immolative linker, then L¹ is a bond; and

L³ is a peptide linker;

wherein the method comprises: reacting Compound X¹:

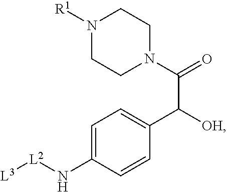

and a compound comprising L¹-D. In some embodiments, the reaction is performed in the presence of p-nitrophenyl chloroformate. In some embodiments, the reaction is performed in the presence of a compound selected from the group consisting of bis(4/p-nitrophenyl) carbonate, phosgene, triphosgene/bis(trichloromethyl carbonate), trichloromethyl chloroformate, N,N′-disuccinimidyl carbonate, and 1,1′-carbonyldiimidazole.

The present disclosure provides for a method of preparing Compound Z:

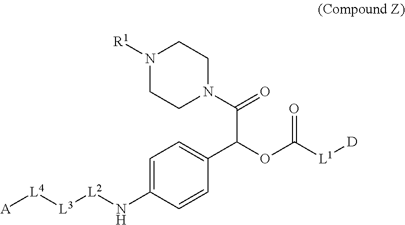

or a salt or solvate or stereoisomer thereof;

wherein:

D is a drug moiety;

R¹ is hydrogen, unsubstituted or substituted C₁₋₃ alkyl, or unsubstituted or substituted heterocyclyl;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit;

comprising: reacting Compound Y¹:

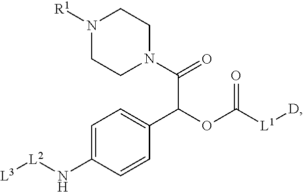

and a compound comprising A-L⁴.

The present disclosure provides for a compound of formula:

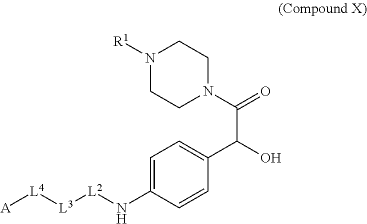

or a salt or solvate or stereoisomer thereof;

wherein:

R¹ is hydrogen, unsubstituted or substituted C₁₋₃ alkyl, or unsubstituted or substituted heterocyclyl;

L² is a bond or a second self-immolative linker;

L³ is a peptide linker;

L⁴ is a bond or a spacer; and

A is an acyl unit.

The present disclosure provides for a compound of formula:

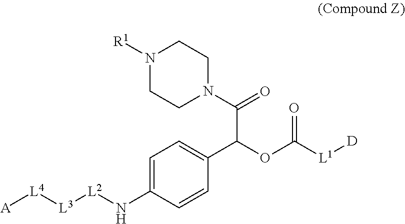

or a salt or solvate or stereoisomer thereof;

wherein:

R¹ is hydrogen, unsubstituted or substituted C₁₋₃ alkyl, or unsubstituted or substituted heterocyclyl;

D is a drug moiety;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond;

L³ is a peptide linker;

L⁴ is a bond or a spacer; and

A is an acyl unit.

The present disclosure provides for a compound of formula:

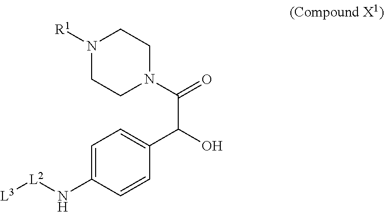

or a salt or solvate or stereoisomer thereof;

wherein:

R¹ is hydrogen, unsubstituted or substituted C₁₋₃ alkyl, or unsubstituted or substituted heterocyclyl;

L² is a bond or a second self-immolative linker; and

L³ is a peptide linker.

The present disclosure provides for a compound of formula:

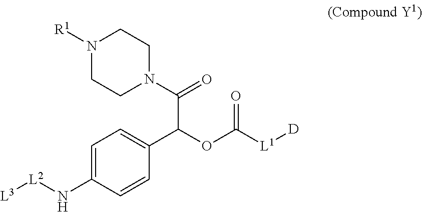

or a salt or solvate or stereoisomer thereof;

wherein:

R¹ is hydrogen, unsubstituted or substituted C₁₋₃ alkyl, or unsubstituted or substituted heterocyclyl;

D is a drug moiety;

L¹ is a bond, a second self-immolative linker, or a cyclization self-elimination linker;

L² is a bond or a second self-immolative linker;
wherein if L¹ is a second self-immolative linker or a cyclization self-elimination linker, then L² is a bond;
wherein if L² is a second self-immolative linker, then L¹ is a bond; and L³ is a peptide linker.

The specification is considered to be sufficient to enable one skilled in the art to practice the invention. Various modifications of the invention in addition to those shown and described herein will become apparent to those skilled in the art from the foregoing description and fall within the scope of the appended claims. All publications, patents, and patent applications cited herein are hereby incorporated by reference in their entirety for all purposes.

### EXAMPLES

The invention can be further understood by reference to the following examples, which are provided by way of illustration and are not meant to be limiting.

### Example 1

### Materials and Methods for Examples 2-5

### Synthesis of Linker-Drug

Synthesis of Compound Tap-18H is shown below in the scheme. Synthesis of intermediate Compounds M and O are also shown in the schemes below.

### Synthesis of Compound M

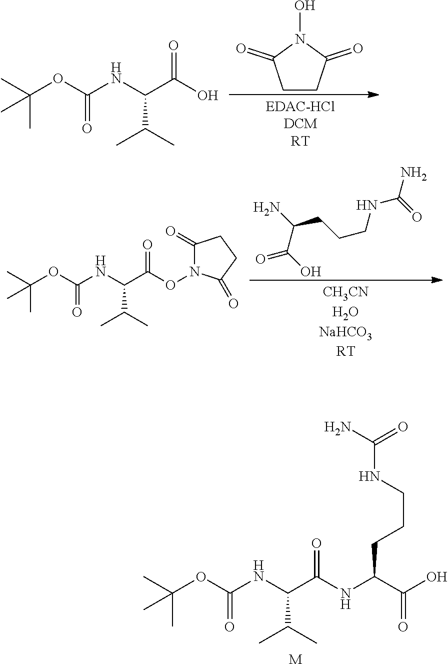

### Synthesis of Compound O

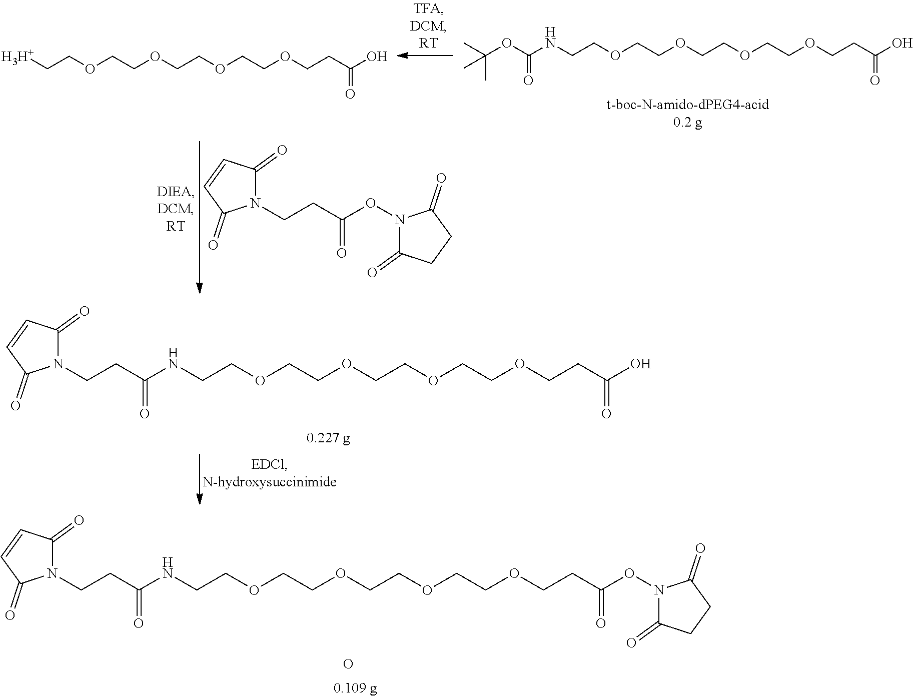 [SMILES: CCOCCOCCOCCOCCC(=O)O ; CC(C)(C)OC(=O)NCCOCCOCCOCCOCCC(=O)O ; O=C(CCN1C(=O)C=CC1=O)ON1C(=O)CCC1=O ; O=C(O)CCOCCOCCOCCOCCNC(=O)CCN1C(=O)C=CC1=O ; O=C(CCN1C(=O)C=CC1=O)NCCOCCOCCOCCOCCC(=O)ON1C(=O)CCC1=O ; O]

### Synthesis of Compound TAP-18H

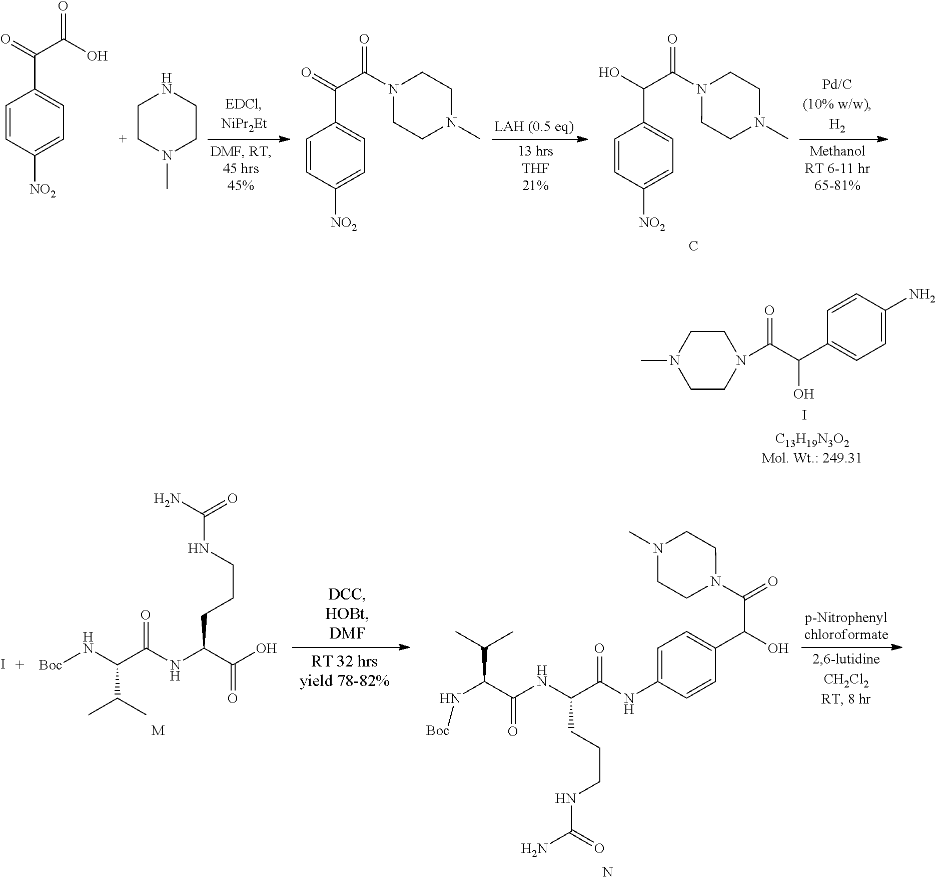

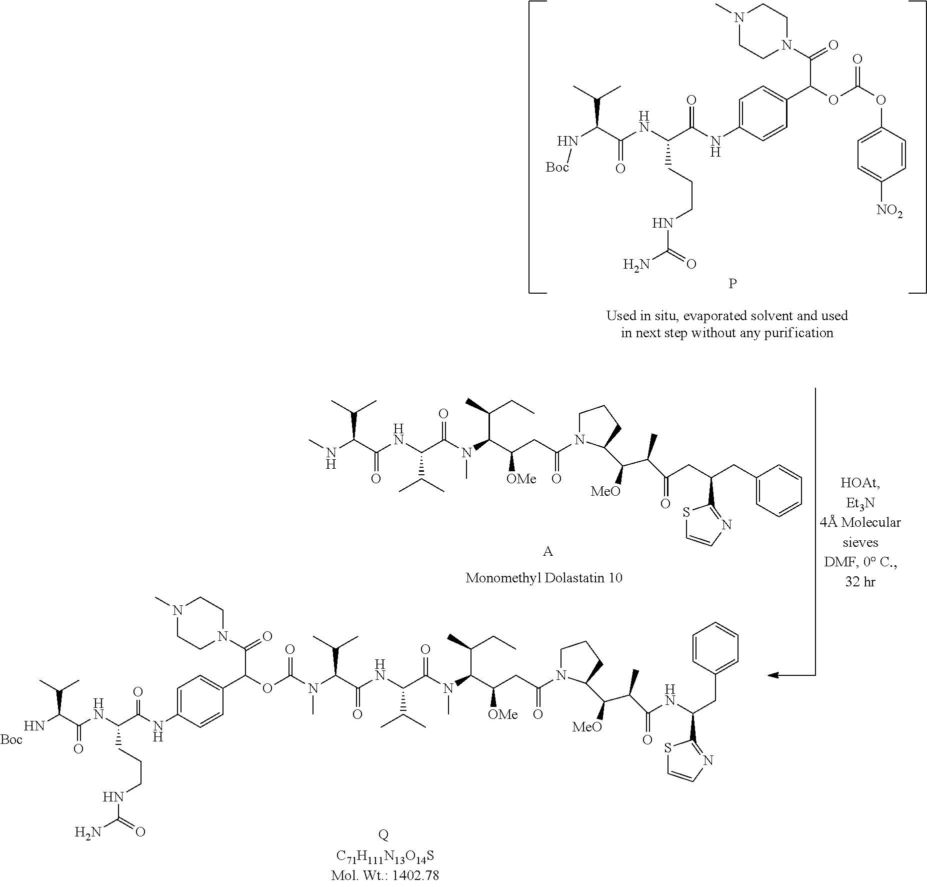

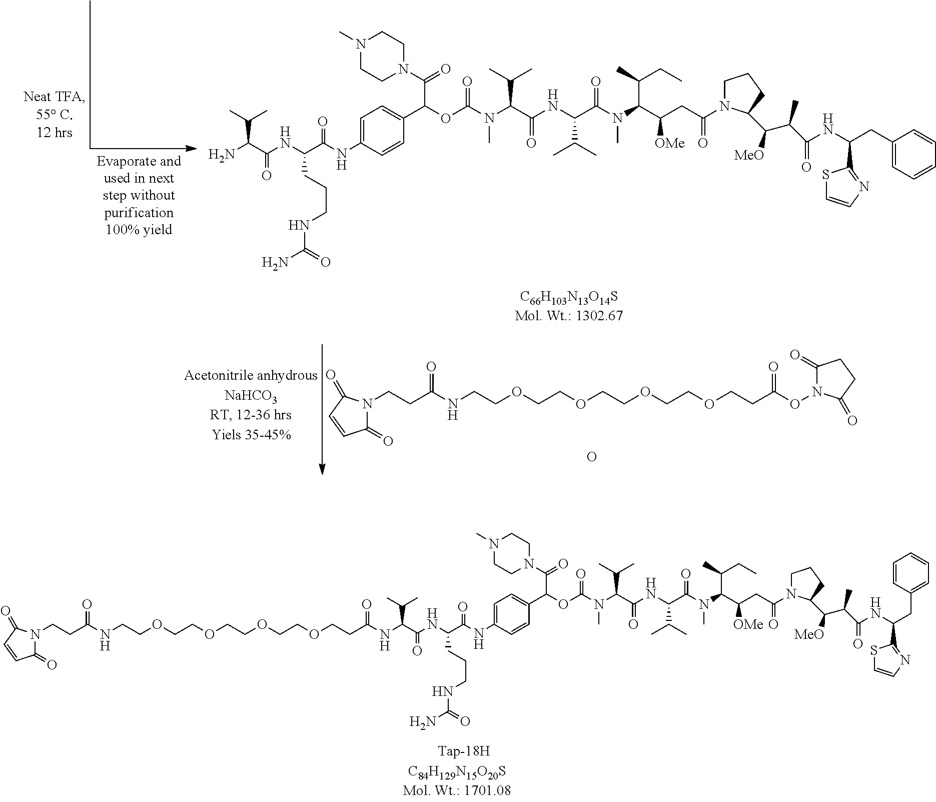

Referring to the scheme of synthesis of Compound Tap-18H, commercially available 4-nitrophenylglyoxylic acid was condensed with N-methylpiperazine using either PCl₅, or EDCI and NiPr₂Et in DMF, or 2-chloro-4,6-dimethoxy-1,3,5-triazine in CH₂Cl₂ and N-methylmorpholine as coupling agent to produce the desired ketoamide. In a typical procedure, a solution of 2-chloro-4,6-dimethoxy-1,3,5-triazine (5 mmol) in CH₂Cl₂ (20 ml), N-methylmorpholine (15 mmol) was added at 0-5° C. under continuous stirring. A white suspension was formed after 30-40 minutes and to this mixture 4-nitrophenylglyoxylic acid in CH₂Cl₂ (10 ml) was added, resulting in the formation of a clear solution. After stirring the mixture for 1 hour, N-methylpiperazine (5 mmol) was added at room temperature. After completion of the reaction (TLC, 10 minutes), the mixture was washed with 10% aqueous NaHCO₃ solution (2×10 ml) followed by H₂O (3×10 ml). The organic layer was dried over anhydrous sodium sulfate and removal of the solvent under reduced pressure furnished a crude product which was further purified by recrystallization or column chromatography (pet. ether:ethyl acetate=8:2).

The ketoamide compound was further reduced by 0.5 equivalent amounts of LiA1H₄ in the presence of THF or DIBAL-H or sodium borohydride to produce the nitro Compound C. [B. P. Bandgar and S. S. Pandit, Tetrahedron Letters 44 (2003) 3855-3858]

Nitro Compound C was reduced to aniline Compound I by either treatment with SnCl₂ or catalytic hydrogenation with Pd/C (10% w/w) as catalyst in methanol at room temperature for about 6-11 hours with yield from 65-81%. It could be obtained through the following procedures using MultiMaxIR system with an RB04-50 Reactor B. The reactor was filled initially with 35 ml of methanol, 0.03 mg of 10% Pd/C and 0.0252 mol of nitro Compound C and the hydrogen was add in the reactor up to pressure at 6.3 bar (H₂, const.).

Referring to the scheme of synthesis of Compound M, Boc-protected L-valine was treated with N-hydroxysuccinimide and EDAC-HCl in DCM or N-hydroxysuccinimide and EDC in DCM to give the succinimide ester. This activated ester was reacted with L-Citrulline and CH₃CN, H₂O, NaHCO₃ to furnish Boc-protected Compound M.

Referring to the scheme of synthesis of Compound Tap-18H, aniline Compound I was coupled with Boc-protected Compound M by means of either DCC/HOBt in DMF at room temperature for 32 hours to give Compound N (yield 78-82%), or with PS-carbodiimide, in which reaction the synthesis of Compound N was carried out starting from 100 mg of Compound M with 1.5 equivalents of aniline Compound I in the presence of two equivalents of PS-carbodiimide and 1.7 equivalents of HOBt in DCM for 24 hours. Analysis by LC/MS showed the peak with the desired mass and approximately 50-60% conversion.

The coupled product Compound N was then reacted with 4-nitrophenyl chloroformate in the presence of 2,6-lutidine in DCM at RT for 8 hours to yield carbonate Compound P, LC/MS showed the peak with the desired mass.

Treatment of carbonate Compound P with monomethyl Dolastatin 10 in the presence of HOAt and Et₃N in DMF resulted in the formation of Compound Q.

Referring to the scheme of synthesis of Compound 0, β-alanine was treated with maleic anhydride in DMF and the acid so obtained was reacted with N-hydroxysuccinimide (NHS) under DCC coupling to give NHS-ester. The BOC protective group in commercially available t-boc-N-amido-dPEG4-acid was removed by treatment with TFA to give the TFA salt of the amine, which was reacted with previously synthesized NHS ester. The carboxylic acid so obtained was isolated and was coupled with N-hydroxysuccinimide using EDCI to furnish NHS ester Compound O.

Referring to the scheme of synthesis of Compound Tap-18H, the Boc-group in Compound Q was removed with TFA and the free amine was coupled with NHS ester Compound O in anhydrous acetonitrile and NaHCO₃ at room temperature for 12-36 hours to produce the final product Tap-18H with yield of 35-45%.

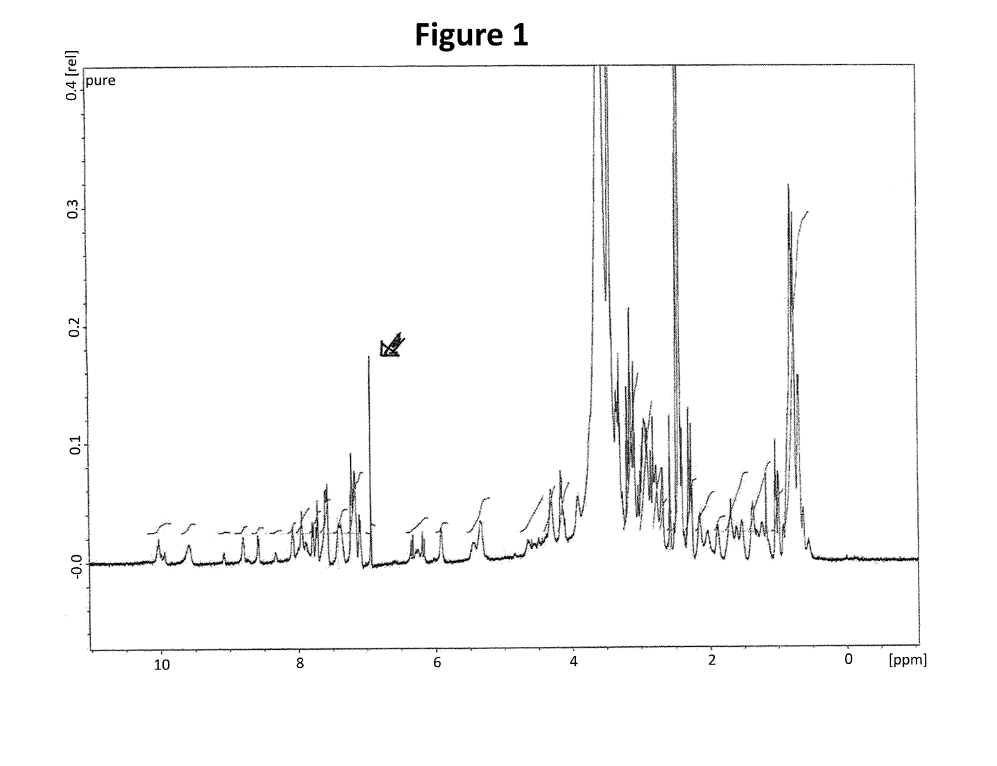

FIG. 1 shows an NMR spectrum of Tap-18H.

### Synthesis of Compound TAP-18Hr1

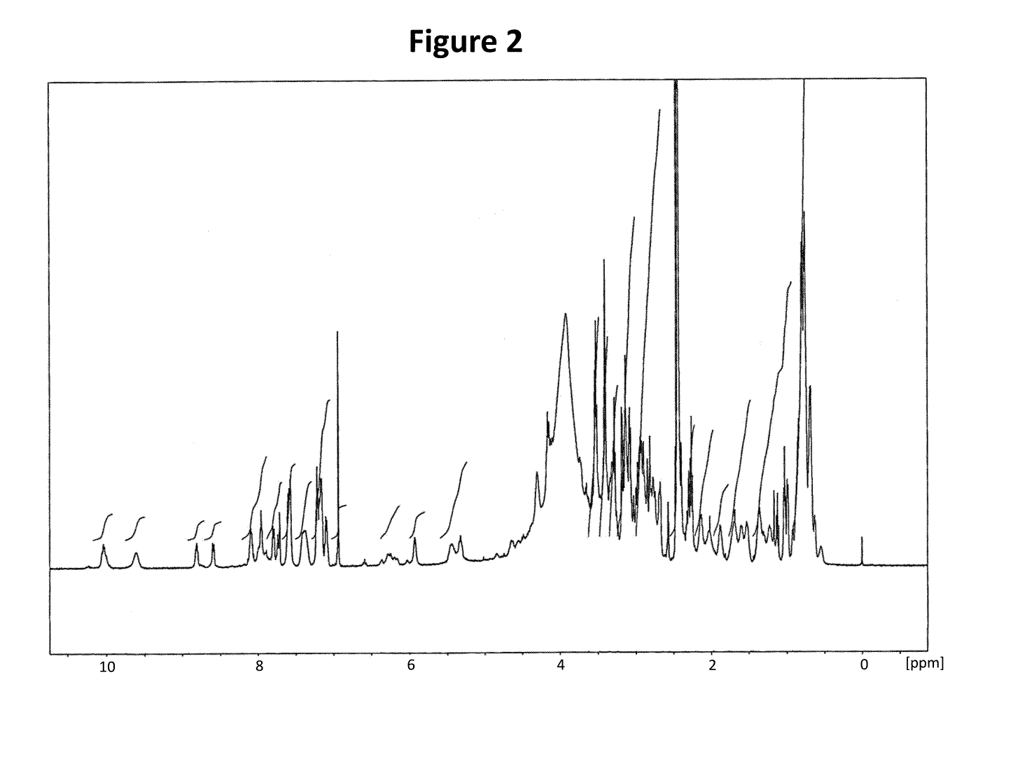

Tap-18Hr1 was synthesized with the formula shown below. FIG. 2 shows NMR spectrum of Tap-18Hr1.

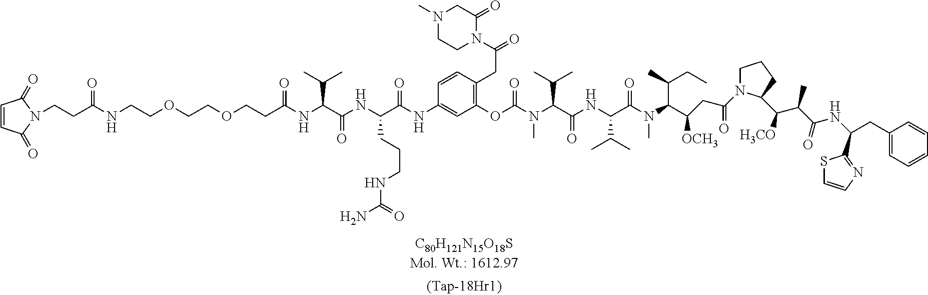 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C(=O)Oc1cc(NC(=O)[C@H](CCCNC(N)=O)NC(=O)[C@@H](NC(=O)CCOCCOCCNC(=O)CCN2C(=O)C=CC2=O)C(C)C)ccc1CC(=O)N1CCN(C)CC1=O)C(C)C]

### Synthesis of Compound TAP-18Hr2

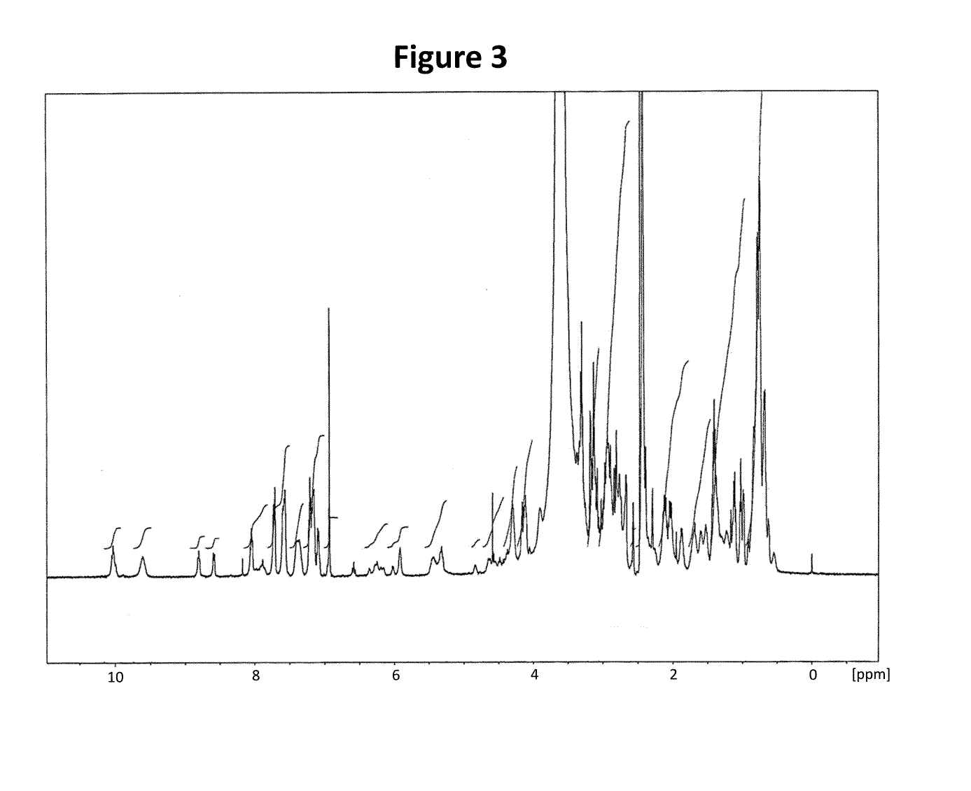

Tap-18Hr2 was synthesized with the formula shown below. FIG. 3 shows NMR spectrum of Tap-18Hr2.

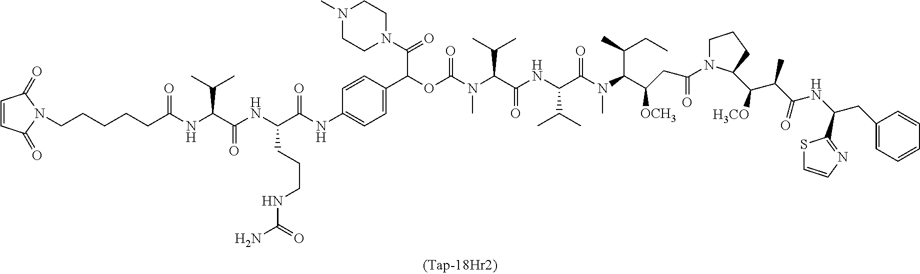 [SMILES: CC[C@H](C)[C@@H]([C@@H](CC(=O)N1CCC[C@H]1[C@H](OC)[C@@H](C)C(=O)N[C@@H](Cc1ccccc1)c1nccs1)OC)N(C)C(=O)[C@@H](NC(=O)[C@H](C(C)C)N(C)C(=O)OC(C(=O)N1CCN(C)CC1)c1ccc(NC(=O)[C@H](CCCNC(N)=O)NC(=O)[C@@H](NC(=O)CCCCCN2C(=O)C=CC2=O)C(C)C)cc1)C(C)C]

### Cell Lines

The human B-cell lymphoma cells Daudi (Bioresource Collection and Research Center (BCRC), Cat. No. 60192), Raji (ATCC, Cat. No. CCL-86), Ramos (BCRC, Cat. No. 60252), RL(ATCC, Cat. No. CRL-2261) were cultured in RPMI Medium 1640 (GIBCO, Cat. No. 22400) supplemented with 10% FBS (GIBCO, Cat. No. 26140), 100 U/mL penicillin/100 μg/mL streptomycin (GIBCO, Cat. No. 15140) and 1 mM sodium pyruvate (GIBCO, Cat. No. 11360). The human acute lymphoblastic leukemia cells REH (DSMZ, Cat. No. ACC 22) were cultured in RMPI Medium 1640 (GIBCO, Cat. No. 22400) supplemented with 20% FBS (GIBCO, Cat. No. 26140), 100 U/mL penicillin/100 μg/mL streptomycin (GIBCO, Cat. No. 15140). The human acute lymphoblastic leukemia cells NALM-6 (DSMZ, Cat. No. ACC 128) were cultured in RPMI Medium 1640 (GIBCO, Cat. No. 22400) supplemented with 10% FBS (GIBCO, Cat. No. 26140), 100 U/mL penicillin/100 μg/mL streptomycin (GIBCO, Cat. No. 15140). The human T-cell leukemia cells Jurkat (BCRC, Cat. No 60424) were cultured in RPMI Medium 1640 (GIBCO, Cat. No. 22400) supplemented with 10% FBS (HyClone, Cat. No. SH30071.03) and 100 U/mL penicillin/100 μg/mL streptomycin (GIBCO, Cat. No. 15140).

Generation of hLL2-Wild Type Antibody

The VH and VL fragments of hLL2, as shown in Table 5, were inserted into pcDNA5-FRT-hIgG1κ or pcDNA5-FRT-hIgG4pκ vector via NheI site and AvrII site for heavy chain and light chain, respectively. The completely assembled plasmid hLL2/pcDNA5-FRT-hIgG1 or hLL2/pcDNAS-FRT-hIgG4p, containing both the heavy chain and light chain genes of hLL2, was used to express hLL2 antibody.

TABLE 5
Amino Acid Sequences of hLL2 anti-CD22 antibody

| SEQ | |
|---|---|
| ID | |
| NO. | DESCRIPTION |
| 1 | Amino acid sequence of hLL2 light chain variable region |
| 2 | Amino acid sequence of hLL2 heavy chain variable region |

(hLL2-light chain variable region)
SEQ ID NO: 1
DIQLTQSPSSLSASVGDRVTMSCKSSQSVLYSANHKNYLAWYQQKPGKAPK
LLIYWASTRESGVPSRFSGSGSGTDFTFTISSLQPEDIATYYCHQYLSSWT
FGGGTKLEIK
(hLL2 heavy chain variable region)
SEQ ID NO: 2
QVQLVQSGAEVKKPGSSVKVSCKASGYTFTSYWLHWVRQAPGQGLEWIGYI
NPRNDYTEYNQNFKDKATITADESTNTAYMELSSLRSEDTAFYFCARRDIT
TFYWGQGTTVTVSS

Generation of hLL2-Cysteine Variants

Cysteine residue was introduced into hLL2-antibody with site-directed mutagenesis method. Briefly, mutagenesis was performed by overlapping PCR. Specific alternation in the desired base can be introduced by incorporating nucleotide changed primers. As the primers were extended, the mutation was created in the resulting amplicon. The mutation position (EU numbering) and flanking sequences of amino acids are listed in Table 6 below. T155C, S157C, S165C, T169C and S442C were used as examples in the further studies.

TABLE 6
Mutation Position (EU Numbering) and Flanking
Sequences of Amino Acids

| EU | | SEQ ID |
|---|---|---|
| numbering | Flanking Sequence | NO: |
| V205C (CL) | EVTHQGLSSPCTKSFNRGEC | 57 |
| T155C | GCLVKDYFPEPVCVSWNSGALTSGV | 58 |
| (CH1) | (hIgG1~4) | |
| S157C (CH1) | LVKDYFPEPVTVCWNSGALTSGVHT | 59 |
| | (hIgG1~4) | |
| S165C (CH1) | PVTVSWNSGALTCGVHTFPAVLQSS | 60 |
| | (hIgG1~4) | |
| T169C (CH1) | SWNSGALTSGVHCFPAVLQSSGLYS | 61 |
| | (hIgG1~4) | |
| T197C (CH1) | VVTVPSSSLGTQCYICNVNHKPSNT (hIgG1) | 62 |
| | VVTVPSSNFGTQCYTCNVDHKPSNT (hIgG2) | 63 |
| | VVTVPSSSLGTQCYTCNVNHKPSNT (hIgG3) | 64 |
| | VVTVPSSSLGTKCYTCNVDHKPSNT (hIgG4) | 65 |
| S442C (CH3) | EALHNHYTQKSLCLSPGK (hIgG1, hIgG2) | 66 |
| | EALHNRFTQKSLCLSPGK (hIgG3) | 67 |
| | EALHNHYTQKSLCLSGK (hIgG4) | 68 |

Production of Stable Cell Lines Expressing hLL2 Antibodies

Wild type and variants of hLL2-Cysteine (hLL2-Cys) were stably expressed and produced in Flp-In CHO cells (Invitrogen, Cat. No: R758-07). The DNA sequences of cysteine substituted antibody variants were inserted to pcDNA5/FRT vector (Invitrogen, Cat. No: V6010-20) and co-transfected with pOG44 (Invitrogen, Cat. No V6005-20) following the standard procedure provided by vendor. The culture supernatants of the established cell lines were collected and purified with protein A sepharose beads (GE Healthcare, Cat. No: 17-5280-04). The purified proteins were analyzed with both SDS-PAGE and size exclusion chromatography to ensure the quality of antibodies.

Conventional Conjugation of hLL2-IgG1 Antibody

hLL2-IgG1 antibody was reduced with 2.25 equivalents of TCEP (Acros Organics, Cat. No: 363830100) in 0.025 M sodium borate pH 8, 0.025 M NaCl, 1 mM DTPA (Sigma-Aldrich, Cat. No: D6518) for 2 hours at 37° C. The protein concentration was quantified using an absorbance value of 1.62 at 280 nm for a 1.0 mg/mL solution, and the molar concentration determined using a molecular weight of 146,514 g/mol. The concentration of mAb-cysteine thiols produced was determined by titrating with DTNB (Thermo Scientific™ Pierce™, Cat. No.: 22582). Typically 3 thiols/mAb was obtained. Partially reduced antibody was alkylated with 1.0 molar of maleimidocaproyl-drugs/mAb-cysteine thiol or maleimido-drugs/mAb-cysteine thiol. The alkylation reaction was performed at 4° C. for 12-16 hours. Cysteine (1 mM final) was used to quench any unreacted, excess maleimidocaproyl-drugs or maleimido-drugs. The Tap18Hr1 conjugation mixture was first diluted 5 fold with binding buffer, 10 mM sodium phosphate, 10 mM NaCl, 5% DMSO, pH 7.0, and applied to a hydroxyapatite column (Macroprep ceramic type 140 μm, BioRad, Hercules, Calif.) at loading capacity of 1 mL hydroxyapatite per 20 mg of conjugated antibody. The column was previously equilibrated with 5 column volumes of binding buffer. Following sample application, the column was washed with 3 column volumes of binding buffer and then equilibrated with 5 column volumes of 10 mM sodium phosphate, 10 mM NaCl, pH 7.0. The binding ADC was then eluted with 200 mM sodium phosphate, 10 mM NaCl, pH 7.0. Following elution, the buffer was changed to Dulbecco's phosphate buffered saline using HiPrep™ 26/10 Desalting column (optional).

Site-Specific Conjugation of hLL2-Cys Variants

To specifically conjugate the linker payload (i.e. the molecule comprising the linker connected to the drug moiety) on the introduced cysteine, a reducing/oxidation procedure was used. To remove cysteine or glutathione on the introduced cysteine site which could have occurred during culture condition, hLL2-Cys variant was first treated with 10-15 fold molar excess of TCEP (Acros Organics, Cat. No: 363830100) at 37° C. for 2-5 hours in PBS containing 1 mM DTPA (Sigma-Aldrich, Cat. No: D6518). The antibody was then re-oxidized with dehydroascorbic acid (DHA) (Sigma-Aldrich, Cat.No:261556) with 20-30 fold molar excess over protein at room temperature for 3-5 hours to ensure the re-formation of inter-chain disulfide bonds. The maleimide-linked drug payload, Tap18Hr1, was then added to react with free-thiols on the processed antibody. The excess payload was quenched with N-acetyl-L-cysteine (Sigma-Aldrich, Cat. No:A7250) and CHT ceramic hydroxyapatite beads (Bio-Rad, Cat.No:157-0040) were used to purify the conjugated antibody.

### Drug Antibody Ratio (DAR) Determination by Reverse Phase HPLC Analysis

A method consisting of reducing and denaturing reversed-phase HPLC (RP-HPLC) was developed to separate and quantify various light and heavy chain species to determine the DAR of conjugated ADC. Prior to HPLC analysis, conjugate sample was treated with 6M guanidine hydrochloride and 20 mM DTT under 50° C. heating for 15 mins. 100 μg of the treated conjugate sample was applied to PLRP-S column (2.1×150 mm, 1000 Å, Aligent). The flow rate was set at 0.8 mL/min and the column temperature was set at 80° C. constantly throughout the analysis. Solvent A was 0.05% trifluoroacetic acid in Milli Q water and solvent B was 0.04% trifluoroacetic acid in acetonitrile. The method consisted of the following: Isocratic 25% B for 3 ml, a 25 ml linear gradient to 50% B, a 2 ml linear gradient to 95% B, a 1 ml linear gradient to 25% B, and isocratic 25% B for 2 ml. In this method, pretreatment of the ADC with an excess of DTT breaks the inter- and intra-chain disulfides and allows separation of light chain with 0 or 1 drug (L0 and L1) from heavy chain with 0, 1, 2, or 3 drugs (H0, H1, H2, and H3). Peaks of each separated species were assigned by their elution time and UV spectra (the A248/280 ratio increases with drug loading). The calculated DAR based on the area of individual peak in the RP-HPLC profile for the tested ADCs were 2.7-3.5 and 1.58-2.01 for conventional conjugation and site-specific conjugation, respectively.

Binding of hLL2-Cys-Variants and the Tap18Hr1 Conjugates to Cancer Cells

6×10⁴˜1×10⁵ cells were seeded per well in a v-bottomed 96-well plate and incubated with 100 μl of the unconjugated Abs or the ADCs at titrated concentrations or isotype control antibody human IgG at 10 μg/mL. After 30-60 minutes of incubation at 4° C., cells were washed once with 200 μl FACS buffer (lx PBS containing 1% FBS), stained with 100 μl of 1 μg/ml goat F(ab′)2-anti-human IgG (H+L)-RPE (Southern Biotech, Cat. No. 2043-09) in FACS buffer and then incubated at 4° C. for 30˜60 min Cells were washed once with FACS buffer and analyzed by flow cytometer (BD LSR, BD Life Sciences).

### In Vitro WST-1 Cytotoxicity Assay

B-cell lymphoma cells (Daud, Raji, Ramos, RL), acute lymphoblastic leukemia (REH) and Jurkat cells were seeded 2×10⁴ and 2.5×10⁴ cells per well, respectively, on 96-well microtiter plates. Tap18Hr conjugated ADCs or unconjugated antibodies were added in triplicates or 6 replicates at the final indicated concentrations in a final volume 200 μL/well. Cells were then incubated at 37° C. and 5% CO₂, and cell viability was detected at 72 or 96 hours by cell proliferation reagent WST-1 (Roche, Cat. No. 11644807001) following manufacturer's instructions. Briefly, at the end of incubation 100 μL of medium was withdrawn and 10 μL/well of WST-1 was added to the tested cell line. After optimal color development (when OD₄₅₀ of untreated control≧1.5), absorbance at 450 nm (OD₄₅₀ value) was measured by spectrophotometer (Molecular Devices, VERSAmax microplate reader). The mean of the replicates was obtained and background (medium control) was subtracted. The resultant OD₄₅₀ values were then used to calculate % inhibition according to the following formula: [OD₄₅₀ untreated−OD₄₅₀ sample]/[OD₄₅₀ untreated]*100.

### ADC Treatment in Cancer Xenograft Model

Daudi and Ramos Treated with hLL2-Tap18Hr1

To establish a subcutaneous xenograft model, 1×10⁷ Daudi or Ramos cells were implanted into the right flank of female C.B-17 SCID mice (Lasco, Taipei, Taiwan). When the average tumor volume reached 100 mm³, mice were randomly grouped and administered with vehicle or ADC intravenously at 3 mg/kg in 100 μL (marked as day 1). Tumor volume was measured twice weekly with a caliper in two perpendicular dimensions, and calculated according to the formula (0.52*length*width*width).

Ramos Treated with hLL2-Cys Variants Conjugated with Tap18Hr1

To establish a subcutaneous xenograft model, 1×10⁷ Ramos cells were implanted into the right flank of female C.B-17 SCID mice (Lasco, Taipei, Taiwan). When average tumor volume reached 115 mm³, mice were randomly grouped (marked as day 1) and administered with vehicle or ADC intravenously at equivalent drug dose of 8.9 μg/kg in 100 μL (i.e., the injected dose was adjusted with respect to the equal amount of monomethyl dolastatin-10 administered). Tumor volume was measured twice weekly with a caliper in two perpendicular dimensions, and calculated according to the formula (0.52*length*width*width).

REH Treated with hLL2-Cys Variants Conjugated with Tap18Hr1

To establish a subcutaneous xenograft model, 1×10⁷ REH cells were mixed 1:1 with Matrigel® and implanted into the right flank of female C.B-17 SCID mice (Lasco, Taipei, Taiwan). When average tumor volume reached ˜100 mm³, mice were randomly grouped (marked as day 1) and administered with vehicle or ADC intravenously at about equivalent drug dose of 8.9 μg/kg (i.e., the injected dose was adjusted with respect to the equal amount of monomethyl dolastatin-10 administered). Tumor volume was measured twice weekly with a caliper in two perpendicular dimensions, and calculated according to the formula (0.52*length*width*width).

### Example 2

### Binding Affinity of Anti-CD22-IgG1/IgG4p-Tap18Hr1 ADC and Anti-CD22-IgG1/IgG4p to CD22-Expressing Cell Lines

hLL2-Tap18Hr1 Binding Activity

The binding affinity of hLL2-IgG1-Tap18Hr1 ADC was evaluated in the CD22 expressing cancer cell lines (Daudi, Raji, Ramos, RL, REH and NALM-6). Data in Table 7 show that Anti-hLL2-IgG1-Tap18Hr1 binds significantly to these cells, suggesting various CD22 expression levels in B-cell lymphoma cell lines: Daudi>Raji>Ramos>RL. Conjugation did not alter the binding affinity to antigen CD22, as hLL2-IgG1-Tap18Hr1 and hLL2-IgG1 bind to each tested B-cell lymphoma cell line with comparable mean fluorescence intensity (MFI). These results demonstrate that hLL2-IgG1-Tap18Hr1 retains antigen reactivity of hLL2-IgG1 and binds to multiple B-cell lymphoma cell lines and ALL cell lines effectively.

TABLE 7
Binding of hLL2-IgG1-Tap18Hr1/hLL2-IgG1 to Cancer Cells

| | | | |
|---|---|---|---|
| | hLL2-IgG1- | | |
| | Tap18Hr1 | hLL2-IgG1 | Isotype control |
| MFI | (3.3 μg/mL) | (3.3 μg/mL) | (10 μ/mL) |

Exp. I

| | | | |
|---|---|---|---|
| Daudi | 1036 | 1081 | 8.3 |
| Raji | 561 | 610 | 6.2 |
| Ramos | 187 | 215 | 5.4 |
| RL | 82.5 | 102 | 12.8 |

Exp. II

| | | | |
|---|---|---|---|
| REH | 104 | 139 | *ND |
| NALM-6 | 143 | 215 | *ND |

*ND: Not determined.

The binding ability of hLL2-Cys variants with or without drug conjugation was evaluated in Daudi and REH cancer cells. Data in Table 8 show that hLL2-IgG1 cysteine variants bind comparably to Daudi cells with hLL2-IgG1 Ab. Data in Table 9 show that hLL2-IgG1 cysteine variants bind comparably to REH cells with hLL2-IgG1 Ab. In addition, the site-specific conjugated hLL2-IgG1 ADCs also retained antigen reactivity of the unconjugated antibody, except hLL2-S442C-IgG1 that displayed slightly lower affinity than other variants.

TABLE 8
Binding of hLL2-IgG1 cvsteine variants to Daucli cells

| | Unconjugated Ab | Tap18Hr1 conjugates |
|---|---|---|
| MFI | (3.3 μg/mL) | (3.3 μg/mL) |
| hLL2-T155C-IgG1 | 1249 | 1234 |
| hLL2-S157C-IgG1 | 1302 | 1233 |
| hLL2-S165C-IgG1 | 1308 | 1227 |
| hLL2-T169C-IgG1 | 1300 | 1370 |
| hLL2-S442C-IgG1 | 1150 | 914 |
| hLL2-IgG1 | 1438 | 1402 |

| | |
|---|---|
| 2^{nd} Ab only | 8.4 |

TABLE 9
Binding of hLL2-IgG1 cysteine variants to REH cells

| | Unconjugated Ab | Tap18Hr1 conjugates |
|---|---|---|
| MFI | (3.3 μg/mL) | (3.3 μg/mL) |
| hLL2-S157C-IgG1 | 135 | 94 |
| hLL2-S442C-IgG1 | ND | 87 |
| hLL2-IgG1 | 139 | 104 |

| | |
|---|---|
| 2^{nd} Ab only | 4.5 |

*ND: Not determined.

Data in Table 10 (binding to Daudi) and Table 11 (binding to REH) show that binding of hLL2-IgG4p cysteine variants is comparable to that of hLL2-IgG4p Ab, but is lower when compared with that of hLL2-IgG1 Ab. This indicates that the decreased binding activity was attributed to IgG4 isotype rather than cysteine mutation. In general, the site-specific conjugated hLL2-IgG4p ADCs retained antigen reactivity of antibody. Similar to IgG1 variants, hLL2-S442C-IgG4p also displayed slightly lower affinity than the other IgG4p variants.

TABLE 10
Binding of hLL2-IgG4p cysteine variants to Daucli cells

| | Unconjugated Ab | Tap18Hr1 conjugates |
|---|---|---|
| MFI | (3.3 μg/mL) | (3.3 μg/mL) |
| hLL2-T155C-IgG4p | 601 | 515 |
| hLL2-T169C-IgG4p | 761 | 625 |
| hLL2-S442C-IgG4p | 681 | 481 |
| hLL2-IgG4p | 792 | *ND |
| hLL2-IgG1 | 1393 | 1403 |

| | |
|---|---|
| Isotype control (10 μg/ml) | 26.1 |

*ND: Not determined.

TABLE 11
Binding of hLL2-IgG4p cysteine variants to REH cells

| | Unconjugated Ab | Tap18Hr1 conjugates |
|---|---|---|
| MFI | (3.3 μg/mL) | (3.3 μg/mL) |
| hLL2-T155C-IgG4p | 79 | 66 |
| hLL2-T169C-IgG4p | 74 | 60 |
| hLL2-S442C-IgG4p | 70 | 52 |
| hLL2-IgG4p | 81 | *ND |
| hLL2-IgG1 | 139 | 104 |

| | |
|---|---|
| Isotype control (10 μg/ml) | 4.5 |

*ND: Not determined.

### Example 3

### In Vitro Cytotoxicity of hLL2-IgG1-Tap18Hr1 ADC and hLL2-IgG1 in CD22-Expressing Cell Lines

The in vitro cytotoxic activity of hLL2-IgG1-Tap18Hr1 was evaluated in the CD22 positive cancer cell lines (Daudi, Raji, Ramos, RL and REH) and a CD22 negative cell line (Jurkat). Cytotoxicity by the naked hLL2-IgG1 antibody was also tested in parallel. (Table 12) At 3.3 and 1.1 μg/mL, hLL2-IgG1-Tap18Hr1 was much more potent than the unconjugated antibody hLL2-IgG1 in killing the CD22 positive B-cell lymphoma cells (Daudi, Raji, Ramos and RL). Among the tested cell lines, the order of sensitivity to ADC was Ramos>Daudi>RL>Raji, not exactly correlating with expression levels shown in Table 7. At 10 μg/ml, hLL2-IgG1-Tap18Hr1 showed higher potency than the unconjugated antibody hLL2-IgG1 in killing CD22 positive acute lymphoblastic leukemia cells REH. No toxicity was observed in the CD22 negative cell line Jurkat. These results demonstrate that hLL2-IgG1-Tap18Hr1 delivered cytotoxic drug to the target cancer cells with antigen specificity.

TABLE 12
In vitro cytotoxic activity of hLL2-IgG1-Tap18Hr1 and hLL2-IgG1

| (% inhibition) | | 3.3 μg/mL | 1.1 μg/mL |
|---|---|---|---|
| Daudi | hLL2-IgG1-Tap18Hr1 | 86.0 | 86.8 |
| | hLL2-IgG1 | 22.3 | 26.6 |
| Raji | hLL2-IgG1-Tap18Hr1 | 69.2 | 62.1 |
| | hLL2-IgG1 | 26.7 | 26.2 |
| Ramos | hLL2-IgG1-Tap18Hr1 | 100.3 | 99.8 |
| | hLL2-IgG1 | 12.9 | 13.1 |
| RL | hLL2-IgG1-Tap18Hr1 | 79.6 | 78.7 |
| | hLL2-IgG1 | 6.4 | 6.0 |
| Jurkat | hLL2-IgG1-Tap18Hr1 | 5.8 | 5.1 |
| | hLL2-IgG1 | 2.8 | 7.9 |

| (% inhibition) | | 10 μg/mL |
|---|---|---|
| REH | hLL2-IgG1-Tap18Hr1 | 34.9 |
| | hLL2-IgG1 | 0 |
| Jurkat | hLL2-IgG1-Tap18Hr1 | 9.0 |
| | hLL2-IgG1 | 18.9 |

The in vitro cytotoxic activity of the site-specific conjugated hLL2-Tap18Hr1 was also evaluated in Daudi, Ramos, REH and NALM-6 cells. Table 13 shows IgG1 variants and Table 14 shows IgG4p variants. At 3.3 and 1.1 μg/mL, the site-specific conjugates were potent in killing the CD22 positive B-cell lymphoma cells (Daudi, Ramos) but not in the CD22 negative cell line (Jurkat). Despite slightly lower binding affinity, hLL2-S442C-IgG1-Tap18Hr1 induced similar degree of cytotoxicity as other cysteine variants in Daudi and Ramos cells. The site-specific conjugates at 10 ug/ml were potent in killing CD22 positive acute lymphoblastic leukemia cells REH and NALM-6 cells, but not CD22-negative Jurkat cells. These results demonstrate that the site-specific conjugated hLL2-Tap18Hr1 ADCs can deliver cytotoxic drug to the target cancer cells with antigen specificity.

TABLE 13
In vitro cytotoxic activity by the
conjugated hLL2 IgG1 Cys variants

| (% inhibition) | | 3.3 μg/mL | 1.1 μg/mL |
|---|---|---|---|
| Daudi | hLL2-T155C-IgG1-Tap18Hr1 | 72.6 | 71.0 |
| | hLL2-S157C-IgG1-Tap18Hr1 | 79.4 | 76.3 |
| | hLL2-S165C-IgG1-Tap18Hr1 | 65.1 | 60.5 |
| | hLL2-T169C-IgG1-Tap18Hr1 | 73.6 | 69.8 |
| | hLL2-S442C-IgG1-Tap18Hr1 | 79.0 | 77.6 |
| Ramos | hLL2-T155C-IgG1-Tap18Hr1 | 88.9 | 84.9 |
| | hLL2-S157C-IgG1-Tap18Hr1 | 93.9 | 90.4 |
| | hLL2-S165C-IgG1-Tap18Hr1 | 79.4 | 72.6 |
| | hLL2-T169C-IgG1-Tap18Hr1 | 91.4 | 85.7 |
| | hLL2-S442C-IgG1-Tap18Hr1 | 96.0 | 92.0 |
| Jurkat | hLL2-T155C-IgG1-Tap18Hr1 | 4.5 | 3.2 |
| | hLL2-S157C-IgG1-Tap18Hr1 | 2.8 | 1.8 |
| | hLL2-S165C-IgG1-Tap18Hr1 | 8.7 | 3.4 |
| | hLL2-T169C-IgG1-Tap18Hr1 | 2.8 | −1.6 |
| | hLL2-S442C-IgG1-Tap18Hr1 | 5.9 | 5.8 |

| (% inhibition) | | 10 μg/mL |
|---|---|---|
| REH | hLL2-T157C-IgG1-Tap18Hr1 | 67.0 |
| | hLL2-S442C-IgG1-Tap18Hr1 | 35.2 |
| NALM-6 | hLL2-T157C-IgG1-Tap18Hr1 | 92.5 |
| | hLL2-S442C-IgG1-Tap18Hr1 | 43.1 |
| Jurkat | hLL2-T157C-IgG1-Tap18Hr1 | 34.9 |
| | hLL2-S442C-IgG1-Tap18Hr1 | 13.0 |

TABLE 14
In vitro cytotoxic activity by the
conjugated hLL2 IgG4p Cys variants

| (% inhibition) | | 3.3 μg/mL | 1.1 μg/mL |
|---|---|---|---|
| Daudi | hLL2-T155C-IgG4p-Tap18Hr1 | 70.2 | 66.2 |
| | hLL2-T169C-IgG4p-Tap18Hr1 | 70.8 | 67.2 |
| | hLL2-S442C-IgG4p-Tap18Hr1 | 70.6 | 65.8 |
| Ramos | hLL2-T155C-IgG4p-Tap18Hr1 | 94.9 | 89.4 |
| | hLL2-T169C-IgG4p-Tap18Hr1 | 93.3 | 89.6 |
| | hLL2-S442C-IgG4p-Tap18Hr1 | 88.9 | 86.9 |
| Jurkat | hLL2-T155C-IgG4p-Tap18Hr1 | 8.8 | 0.6 |
| | hLL2-T169C-IgG4p-Tap18Hr1 | 6.3 | 1.9 |
| | hLL2-S442C-IgG4p-Tap18Hr1 | 6.8 | 7.0 |

| (% inhibition) | | 10 ug/ml |
|---|---|---|
| REH | hLL2-T155C-IgG4p-Tap18Hr1 | 22.4 |
| | hLL2-T169C-IgG4p-Tap18Hr1 | 42.6 |
| | hLL2-S442C-IgG4p-Tap18Hr1 | 21.3 |
| NALM-6 | hLL2-T155C-IgG4p-Tap18Hr1 | 45.3 |
| | hLL2-T169C-IgG4p-Tap18Hr1 | 68.2 |
| | hLL2-S442C-IgG4p-Tap18Hr1 | 28.5 |
| Jurkat | hLL2-T155C-IgG4p-Tap18Hr1 | 9.0 |
| | hLL2-T169C-IgG4p-Tap18Hr1 | 0.9 |
| | hLL2-S442C-IgG4p-Tap18Hr1 | 0.8 |

### Example 4

### Daudi and Ramos Xenograft Models Treated with hLL2-IgG1-Tap18Hr1

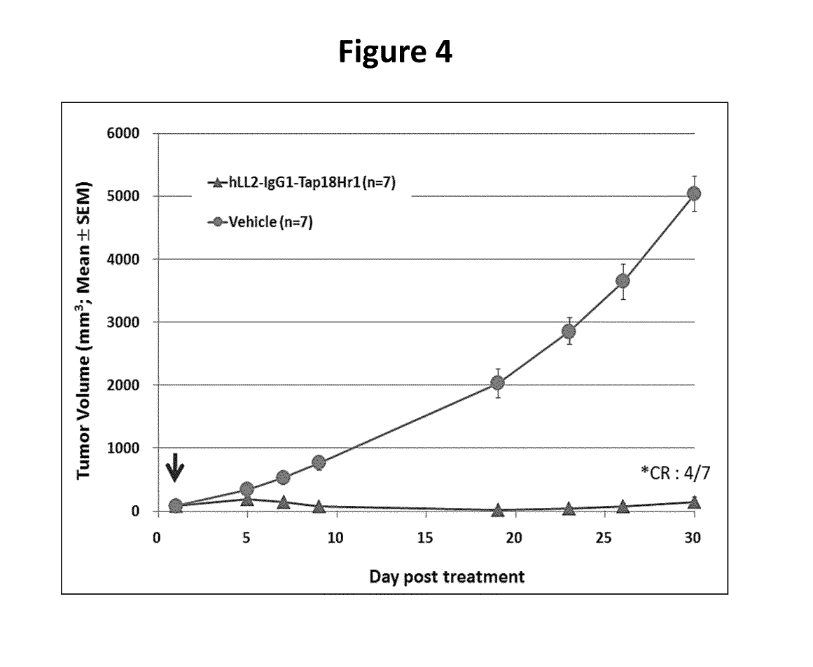

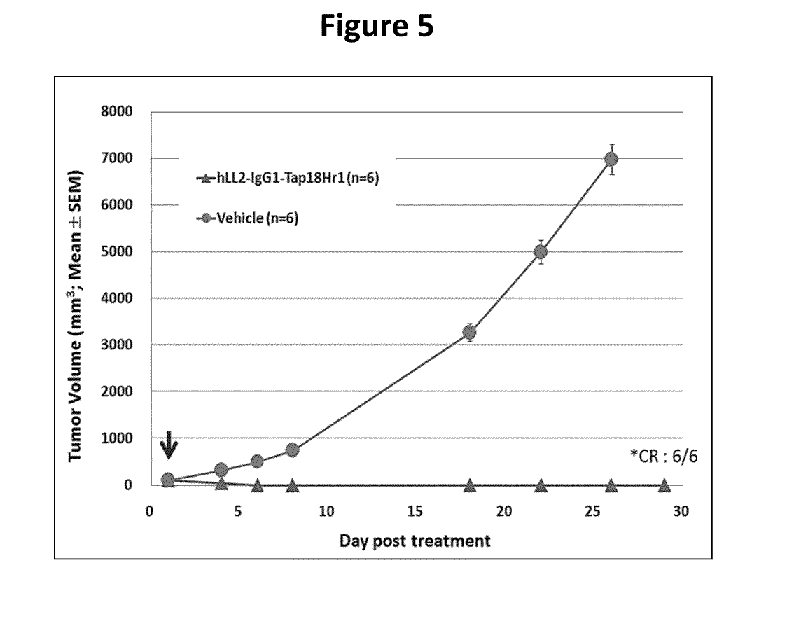

Potency of hLL2-Tap18Hr1-IgG1 was evaluated in vivo in xenograft models derived from the B-cell lymphoma cancer cell lines Daudi and Ramos. When the average inoculated tumor size reached ˜100 mm³, mice were treated intravenously with PBS (vehicle) or a single dose of ADC at 3 mg/kg (marked by the arrow as Day 1 in FIGS. 4-5). In Daudi xenograft (FIG. 4), while tumor of the vehicle group rapidly grew and approached 500 mm³ at day 7, hLL2-IgG1-Tap18Hr1 group showed delayed tumor growth at Day 5, and mean tumor size was further suppressed to <90 mm³ from Day 9. At the end of study, 4 out of 7 mice remained complete tumor regression (Complete regression (CR): Number of CR/number of total.) In the Ramos xenograft (FIG. 5), while tumor of the vehicle group rapidly grew and approached 500 mm³ at Day 6, hLL2-IgG1-Tap18Hr1 led to tumor suppression at Day 5, and all mice (6/6) in this group showed complete regression after Day 12. (Complete regression (CR): Number of CR/number of total.) In both xenograft models, body weight remained unchanged in ADC-treated group and slightly increased in vehicle group due to the weight of tumor burden. Taken together, the data demonstrate that with a single injection, hLL2-IgG1-Tap18Hr1 can effectively inhibit growth of antigen positive tumor grafted in SCID mice.

### Example 5

### Ramos, REH Xenograft Treated with hLL2-Cys Variants Conjugated with Tap18Hr1

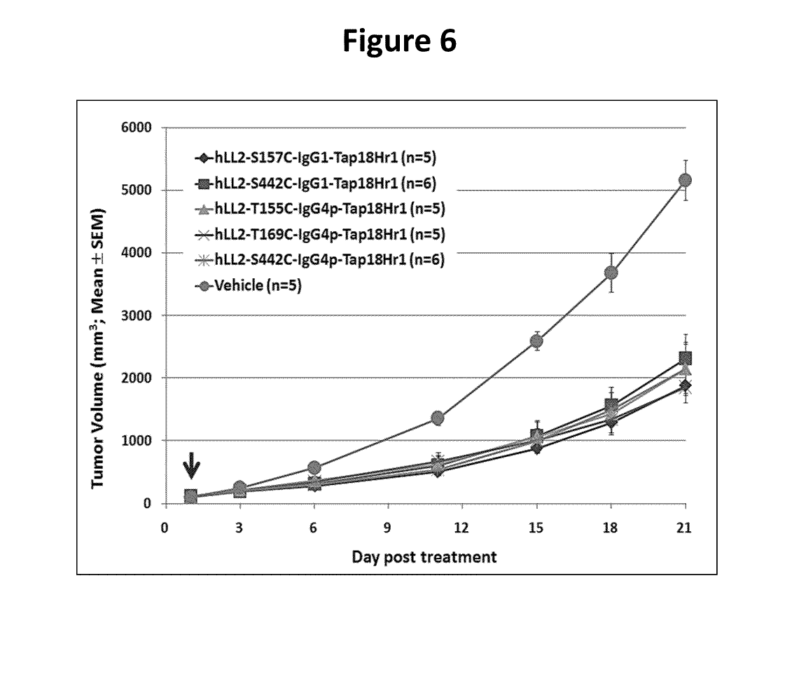

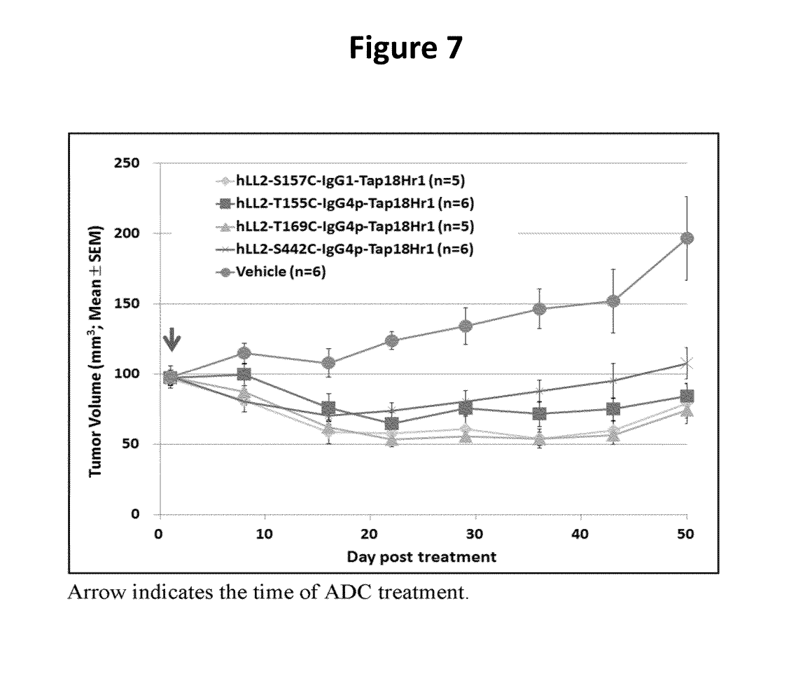

Potencies of the hLL2 site-specific conjugated ADCs were evaluated in vivo in xenograft models derived from the Ramos cell line and REH cell line. In the Ramos xenograft model, when average tumor volume reached 115 mm³, two site-specific conjugated IgG1 variants (S157C, S422C) and three IgG4p variants (T155C, T169C, S442C) were each injected intravenously once at equivalent drug dose 8.9 μg/kg in 100 μL (marked by the arrow as day 1 in FIG. 6). As shown in FIG. 6, all mice treated with site-specific conjugated variants showed significantly delayed tumor growth compared to vehicle group (p value 0.05 since day 6 of treatment). Body weight remained unchanged in ADC-treated group and slightly increased in vehicle group due to the weight of tumor burden. In the REH xenograft model, when average tumor volume reached 100 mm³, one site-specific conjugated IgG1 variant (S157C) and three IgG4p variants (T155C, T169C, S442C) were each injected intravenously once at about equivalent drug dose of 8.9 μg/kg in 100 μL (marked by the arrow as day 1 in FIG. 7). As shown in FIG. 7, all mice treated with site-specific conjugated variants treated showed significantly delayed tumor growth compared to vehicle group (p value 0.05 since day 16 of treatment). Body weight remained unchanged in ADC-treated group and slightly increased in vehicle group due to the weight of tumor burden. The data demonstrate that with a single injection, site-specific conjugated hLL2-Tap18Hr1 ADCs can effectively inhibit growth of antigen positive tumor grafted in SCID mice.

### REFERENCES

1. Carter, P J and Senter, PD. Antibody-drug conjugates for cancer therapy. Cancer J. 2008; 14: 154-169)
2. Teicher, B A. Antibody-drug conjugate targets. Current Cancer Drug Targets 2009, 9: 982-1004.
3. Ducry, L and Stump, B. Antibody-drug conjugates: linking cytotoxic payloads to monoclonal antibodies. Bioconjugate Chem., 2010, 21: 5-13.
4. Koblinski, J E., Ahram, M and Sloane, B F. Unraveling the role of proteases in cancer. Clin. Chem. Acta 2000; 291:113-135.

All patents, patent applications, documents, and articles cited herein are herein incorporated by reference in their entireties.

1. A compound of the formula (I): 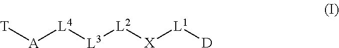
or a salt or solvate or stereoisomer thereof;
wherein:
D is a drug moiety;
T is a targeting moiety which is an antibody that specifically binds to a human CD22;
X is a hydrophilic self-immolative linker;
L¹ is a bond, a second self-immolative linker, or a cyclization self-elimination linker;
L² is a bond or a second self-immolative linker;
wherein if L¹ is a second self-immolative linker or a cyclization self-elimination linker, then L² is a bond;
wherein if L² is a second self-immolative linker, then L¹ is a bond;
L³ is a peptide linker;
L⁴ is a bond or a spacer; and
A is an acyl unit.

2. The compound of claim 1, wherein the compound is of the formula (II): 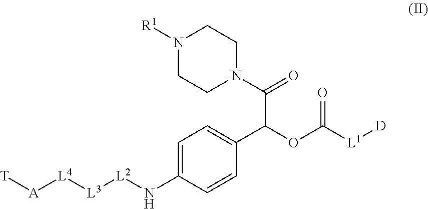
or a salt or solvate or stereoisomer thereof;
wherein:
D is a drug moiety;
T is a targeting moiety which is an antibody that specifically binds to a human CD22;
R¹ is hydrogen, unsubstituted or substituted C₁₋₃ alkyl, or unsubstituted or substituted heterocyclyl;
L¹ is a bond, a second self-immolative linker, or a cyclization self-elimination linker;
L² is a bond or a second self-immolative linker;
wherein if L¹ is a second self-immolative linker or a cyclization self-elimination linker, then L² is a bond;
wherein if L² is a second self-immolative linker, then L¹ is a bond;
L³ is a peptide linker;
L⁴ is a bond or a spacer; and
A is an acyl unit.

3. A compound of the formula (Ia): 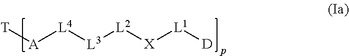
or a salt or solvate or stereoisomer thereof;
wherein:
p is 1 to 20;
D is a drug moiety;
T is a targeting moiety which is an antibody that specifically binds to a human CD22;
X is a hydrophilic self-immolative linker;
L¹ is a bond, a second self-immolative linker, or a cyclization self-elimination linker;
L² is a bond or a second self-immolative linker;
wherein if L¹ is a second self-immolative linker or a cyclization self-elimination linker, then L² is a bond;
wherein if L² is a second self-immolative linker, then L¹ is a bond;
L³ is a peptide linker;
L⁴ is a bond or a spacer; and
A is an acyl unit.

4. The compound of claim 3, wherein the compound is of the formula (IIa): 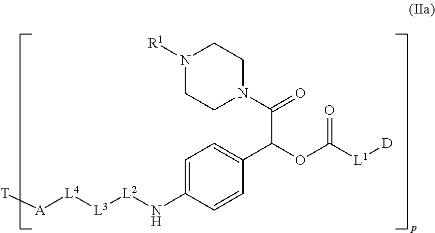
or a salt or solvate or stereoisomer thereof;
wherein:
p is 1 to 20;
D is a drug moiety;
T is a targeting moiety which is an antibody that specifically binds to a human CD22;
R¹ is hydrogen, unsubstituted or substituted C₁₋₃ alkyl, or unsubstituted or substituted heterocyclyl;
L¹ is a bond, a second self-immolative linker, or a cyclization self-elimination linker;
L² is a bond or a second self-immolative linker;
wherein if L¹ is a second self-immolative linker or a cyclization self-elimination linker, then L² is a bond;
wherein if L² is a second self-immolative linker, then L¹ is a bond;
L³ is a peptide linker;
L⁴ is a bond or a spacer; and
A is an acyl unit.

5. The compound of claim 3, wherein p is 1 to 4.

6. The compound of claim 3, wherein L¹ is a bond.

7. The compound of claim 3, wherein L¹ is a second self-immolative linker or a cyclization self-elimination linker.

8. The compound of claim 7, wherein L¹ is an aminobenzyloxycarbonyl linker.

9. The compound of claim 7, wherein L¹ is selected from the group consisting of 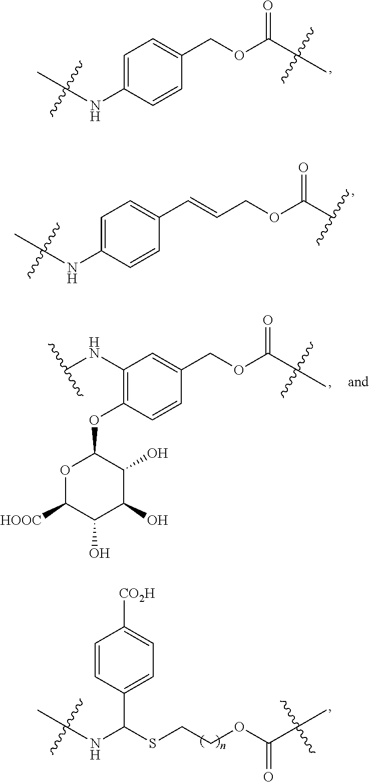
wherein n is 1 or 2.

10. The compound of claim 7, wherein L¹ is selected from the group consisting of

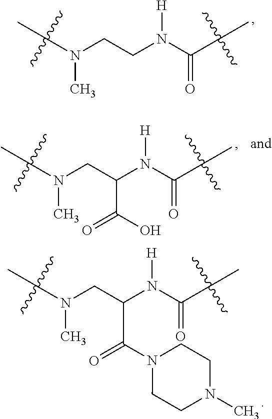

11. The compound of claim 3, wherein L² is a bond.

12. The compound of claim 6, wherein L² is a second self-immolative linker.

13. The compound of claim 12, wherein L² is an aminobenzyloxycarbonyl linker.

14. The compound of claim 12, wherein L² is selected from 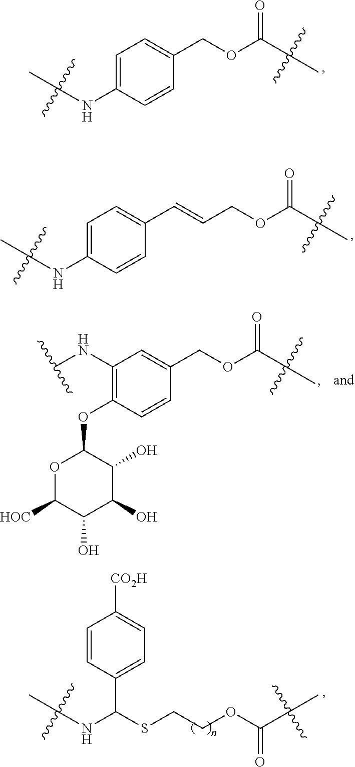
wherein n is 1 or 2.

15. The compound of claim 3, wherein L³ is a peptide linker of 1 to 10 amino acid residues.

16. The compound of claim 15, wherein L³ is a peptide linker of 2 to 4 amino acid residues.

17. The compound of claim 3, wherein L³ is a peptide linker comprising at least one lysine or arginine residue.

18. The compound of claim 3, wherein L³ is a peptide linker comprising an amino acid residue selected from lysine, D-lysine, citrulline, arginine, proline, histidine, ornithine and glutamine.

19. The compound of claim 3, wherein L³ is a peptide linker comprising an amino acid residue selected from valine, isoleucine, phenylalanine, methionine, asparagine, proline, alanine, leucine, tryptophan, and tyrosine.

20. The compound of claim 15, wherein L is a dipeptide unit selected from valine-citrulline, proline-lysine, methionine-D-lysine, asparagine-D-lysine, isoleucine-proline, phenylalanine-lysine, and valine-lysine.

21. The compound of claim 20, wherein L³ is valine-citrulline.

22. The compound of claim 3, wherein L⁴ is a bond.

23. The compound of claim 3, wherein L⁴ is a spacer.

24. The compound of claim 23, wherein the spacer is polyalkylene glycol, alkylene, alkenylene, alkynylene, or polyamine.

25. The compound of claim 23, wherein L⁴ is L^{4a}-C(O), L^{4a}-C(O)—NH, L^{4a}-S(O)₂, or L^{4a}-S(O)₂—NH, wherein each L^{4a} is independently polyalkylene glycol, alkylene, alkenylene, alkynylene, or polyamine.

26. The compound of claim 23, wherein L⁴ is L^{4a}-C(O), wherein L^{4a} is polyalkylene glycol, alkylene, alkenylene, alkynylene, or polyamine.

27. The compound of claim 23, wherein L⁴ is L^{4a}-C(O), wherein L^{4a} is a polyalkylene glycol.

28. The compound of claim 23, wherein L⁴ is L^{4a}-C(O), wherein L^{4a} is a polyethylene glycol.

29. The compound of claim 23, wherein the spacer is of the formula —CH₂—(CH₂—O—CH₂)ₘ—CH₂—C(O)—, wherein m is an integer from 0 to 30.

30. The compound of claim 23, wherein L⁴ is L^{4a}-C(O), wherein L^{4a} is alkylene.

31. The compound of claim 3, wherein A is selected from the group consisting of 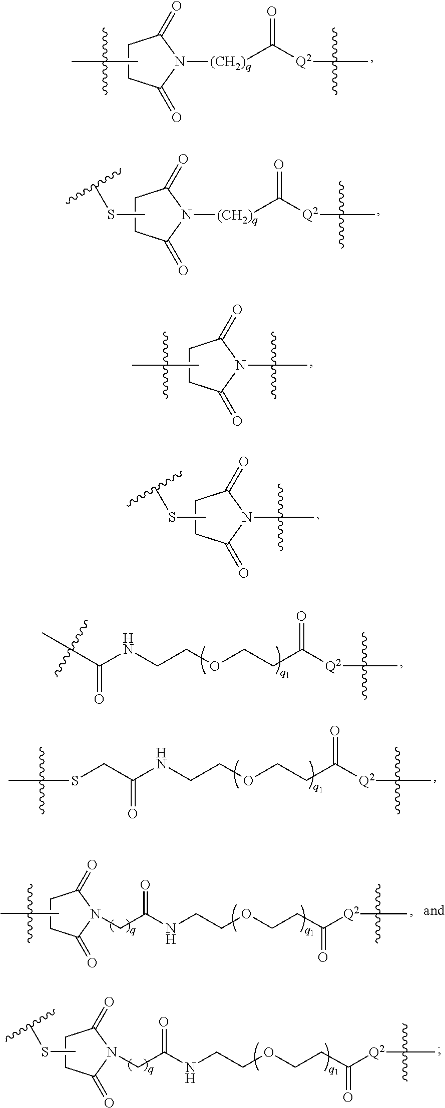
wherein each Q² is NH or O, each q is independently an integer from 1 to 10, and each q₁ is independently an integer from 1 to 10.

32. The compound of claim 31, wherein A is selected from the group consisting of 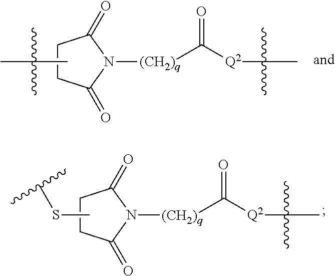
wherein each Q² is independently NH or O and each q is independently an integer from 1 to 10.

33. The compound of claim 32, wherein q is 2, 3, 4, or 5.

34. The compound of claim 3, wherein A is selected from the group consisting of 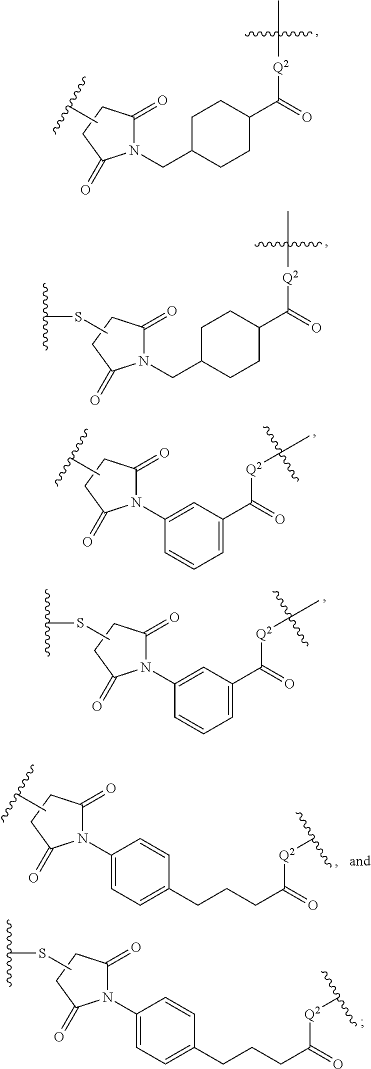
wherein each Q² is independently NH or O.

35. The compound of claim 3, wherein one or more amino acid residues of a heavy chain of the antibody are replaced with a cysteine residue and/or wherein one or more amino acid residues of a light chain of the antibody are replaced with a cysteine residue.

36. The compound of claim 3, wherein the antibody comprises a heavy chain constant region, and wherein one or more amino acid residues in the heavy chain constant region are replaced with a cysteine residue.

37. The compound of claim 36, wherein the antibody comprises a heavy chain constant region, wherein the one or more amino acid residues at positions 155, 157, 165, 169, 188, 197, 199, 208, 209, 211 and 442 in the heavy chain constant region are replaced with a cysteine residue, wherein the numbering is according to the EU index of Kabat.

38. (canceled)

39. The compound of claim 3, wherein the antibody comprises a light chain constant region, and wherein one or more amino acid residues in the light chain constant region of the antibody are replaced with a cysteine residue.

40. The compound of claim 35, wherein D is linked to T via the added cysteine residue.

41. The compound of claim 3, wherein D is an amino-containing drug moiety, wherein the drug is connected to L¹ or X through the amino group.

42. The compound of claim 41, wherein D is duocarmycin, dolastatin, tubulysin, doxorubicin (DOX), paclitaxel, or mitomycin C (MMC), or an amino derivative thereof.

43. The compound of claim 41, wherein D is selected from the group consisting of

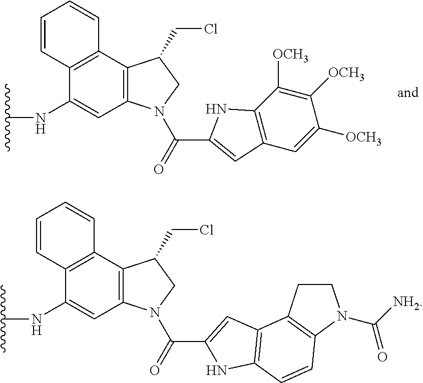 [SMILES: *Nc1cc2c(c3ccccc13)[C@H](CCl)CN2C(=O)c1cc2cc(OC)c(OC)c(OC)c2[nH]1 ; *Nc1cc2c(c3ccccc13)[C@H](CCl)CN2C(=O)c1cc2c3c(ccc2[nH]1)N(C(N)=O)CC3]

44. The compound of claim 41, wherein D is:

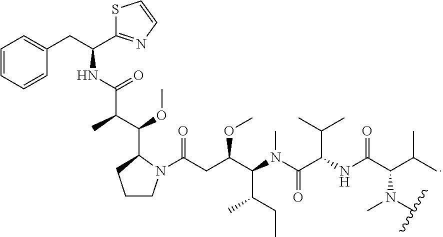 [SMILES: *N(C)[C@H](C(=O)N[C@H](C(=O)N(C)[C@@H]([C@@H](C)CC)[C@@H](CC(=O)N1CCC[C@H]1[C@H](OC)[C@@H](C)C(=O)N[C@@H](Cc1ccccc1)c1nccs1)OC)C(C)C)C(C)C]

45. The compound of claim 3, wherein -A-L⁴-L³-L²- is

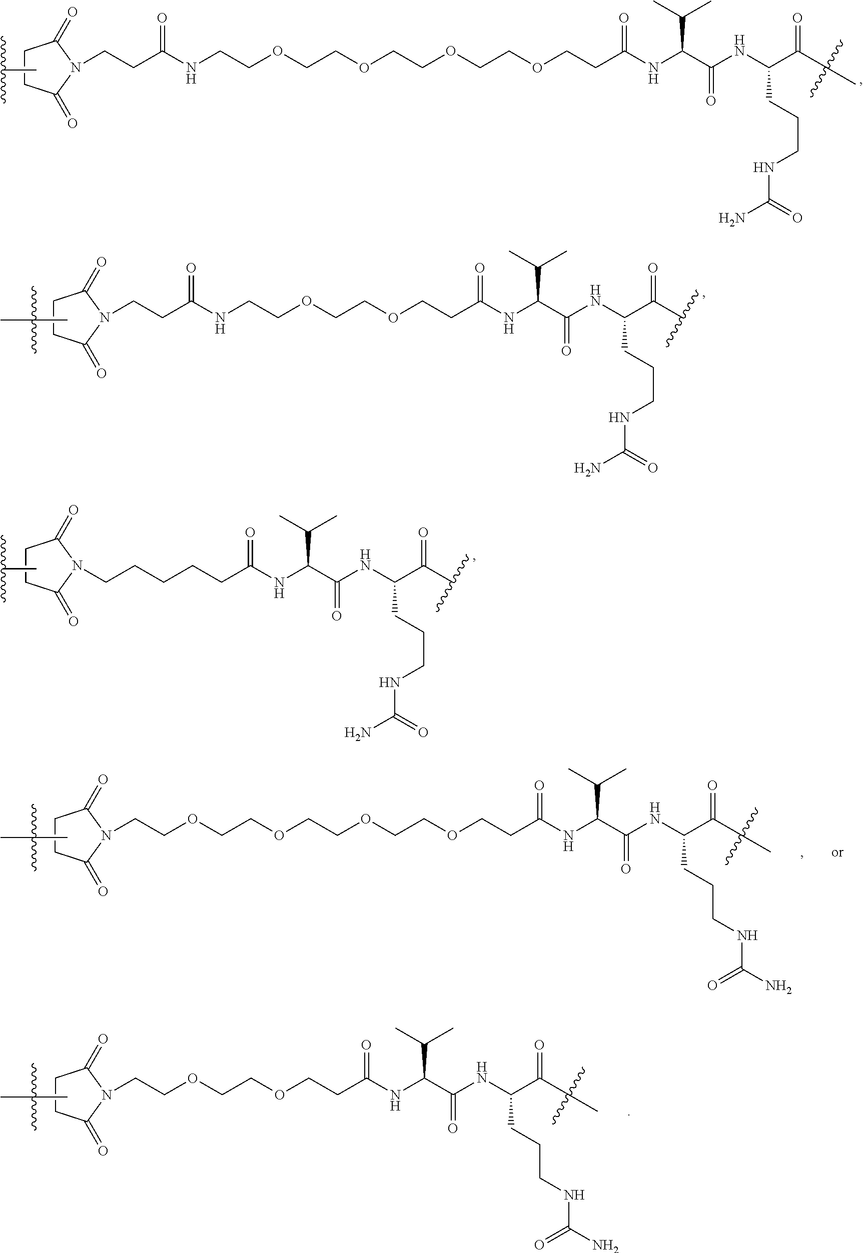

46. The compound of claim 3, wherein -A-L⁴-L³-L²-X-L¹-D is:

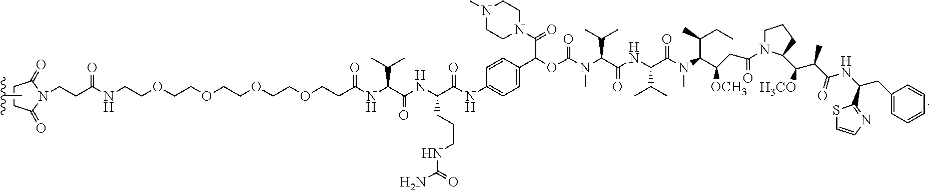

47. The compound of claim 3, wherein -A-L⁴-L³-L²-X-L¹-D is:

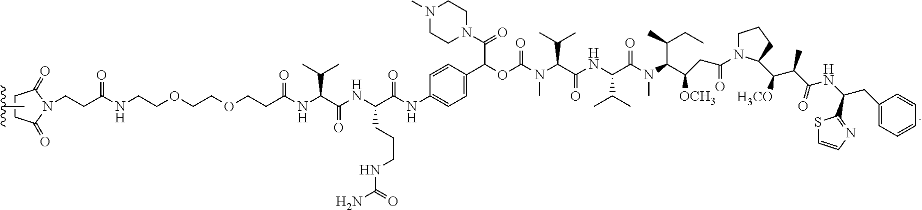

48. The compound of claim 3, wherein -A-L⁴-L³-L²-X-L¹-D is:

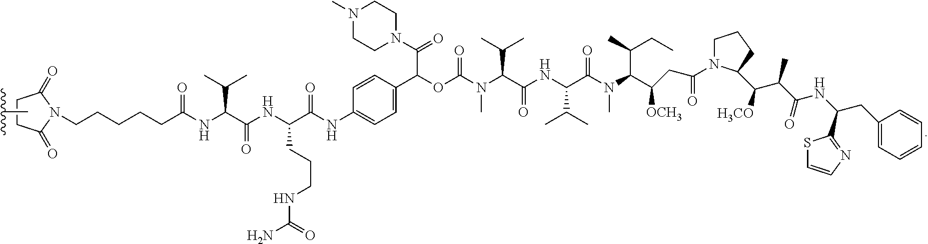

49. The compound of claim 3, wherein the anti-CD22 antibody is a humanized antibody, a chimeric antibody or a human antibody.

50. The compound of claim 3, wherein the anti-CD22 antibody comprises a heavy chain variable region and a light chain variable region, wherein:
(1) the heavy chain variable region comprises the three heavy chain HVRs of the amino acid sequence of SEQ ID NO:2 and/or the light chain variable region comprises the three light chain HVRs of the amino acid sequence of SEQ ID NO:1;
(2) the heavy chain variable region comprises the three heavy chain HVRs of the amino acid sequence of SEQ ID NO:4 and/or the light chain variable region comprises the three light chain HVRs of the amino acid sequence of SEQ ID NO:3;
(3) the heavy chain variable region comprises the three heavy chain HVRs of the amino acid sequence of SEQ ID NO:6 and/or the light chain variable region comprises the three light chain HVRs of the amino acid sequence of SEQ ID NO:5;
(4) the heavy chain variable region comprises the three heavy chain HVRs of the amino acid sequence of SEQ ID NO:8 and/or the light chain variable region comprises the three light chain HVRs of the amino acid sequence of SEQ ID NO:7; or
(5) the heavy chain variable region comprises the three heavy chain HVRs of the amino acid sequence of SEQ ID NO:10 and/or the light chain variable region comprises the three light chain HVRs of the amino acid sequence of SEQ ID NO:9.

51. The compound of claim 3, wherein the anti-CD22 antibody comprises a heavy chain variable region and a light chain variable region, wherein
(1) the heavy chain variable region comprises the amino acid sequence of SEQ ID NO:2 and/or the light chain variable region comprises the amino acid sequence of SEQ ID NO:1;
(2) the heavy chain variable region comprises the amino acid sequence of SEQ ID NO:4 and/or the light chain variable region comprises the amino acid sequence of SEQ ID NO:3;
(3) the heavy chain variable region comprises the amino acid sequence of SEQ ID NO:6 and/or the light chain variable region comprises the amino acid sequence of SEQ ID NO:5;
(4) the heavy chain variable region comprises the amino acid sequence of SEQ ID NO:8 and/or the light chain variable region comprises the amino acid sequence of SEQ ID NO:7; or
(5) the heavy chain variable region comprises the amino acid sequence of SEQ ID NO:10 and/or the light chain variable region comprises the amino acid sequence of SEQ ID NO:9.

52. The compound of claim 3, wherein the antibody comprises a human heavy chain constant region comprising the amino acid sequence of SEQ ID NO:12 or SEQ ID NO:13 and a human light chain constant region comprising the amino acid sequence of SEQ ID NO:11, wherein one or more amino acid residues at positions 155, 157, 165, 169, 188, 197, 199, 208, 209, 211 and 442 in the heavy chain constant region are replaced with a cysteine residue, and/or wherein one or more amino acid residues at positions 147, 188, 200, 201 and 206 in the light chain constant region are replaced with a cysteine residue, wherein the numbering is according to the EU index of Kabat.

53. The compound of claim 3, wherein the antibody is selected from the group consisting of monoclonal antibody, polyclonal antibody, Fab, Fab′, F(ab′)₂, Fv, Fc, chimeric antibody, humanized antibody, human antibody, ScFv, bispecific antibody, multispecific antibody, fusion protein comprising an antibody portion, and single domain antibody.

54. A pharmaceutical composition comprising a compound of claim 3, or a salt or solvate or stereoisomer thereof; and a pharmaceutically acceptable carrier.

55. A method of killing a cell that expresses a human CD22, comprising administering to the cell an effective amount of the compound of claim 3, or a salt or solvate or stereoisomer thereof.

56-57. (canceled)

58. A method of treating cancer in an individual comprising administering to the individual an effective amount of a compound of claim 3, or a salt or solvate or stereoisomer thereof.

59-60. (canceled)

61. A kit comprising a compound of claim 3, or a salt or solvate or stereoisomer thereof.

62. (canceled)

63. A process for making a compound of claim 2, or a salt or solvate or stereoisomer thereof;
comprising reacting an antibody with Compound Z: 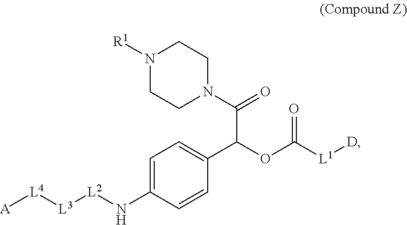
or a salt or solvate or stereoisomer thereof.

64. A process for making a compound of claim 4, or a salt or solvate or stereoisomer thereof;
comprising reacting an antibody with Compound Z: 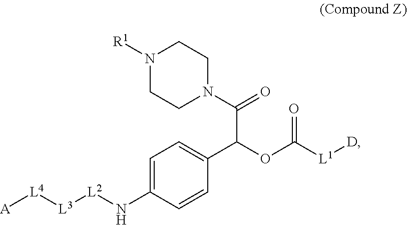
or a salt or solvate or stereoisomer thereof.

65-75. (canceled)

76. A compound, or a salt or solvate or stereoisomer thereof, wherein the compound is prepared by a process according to claim 63, wherein the antibody comprises one or more sulfhydryl groups.

77. (canceled)

78. The compound of claim 39, wherein the antibody comprises a light chain constant region, wherein the one or more amino acid residues at positions 147, 188, 200, 201 and 206 in the light chain constant region are replaced with a cysteine residue, and wherein the numbering is according to the EU index of Kabat.
